(19) United States
(12) Patent Application Publication
(10) Pub. No.: US 2026/0137906 A1
(43) Pub. Date: May 21, 2026

(54) STEERABLE DELIVERY CATHETERS

(71) Applicant: Edwards Lifesciences Corporation, Irvine, CA (US)

(72) Inventors: Peleg Harel, Haifa (IL); Eitan Atias, Tel Aviv (IL); Ofir Witzman, Harish (IL); Halit Yaakobovich, Netanya (IL); Dmitry Pinhasov, Yokneam Illit (IL); David Maimon, Atlit (IL)

(73) Assignee: Edwards Lifesciences Corporation, Irvine, CA (US)

(21) Appl. No.: 19/442,790

(22) Filed: Jan. 7, 2026

Related U.S. Application Data
(63) Continuation of application No. PCT/US2024/038983, filed on Jul. 22, 2024.
(60) Provisional application No. 63/528,945, filed on Jul. 26, 2023.
(60) Provisional application No. 63/631,362, filed on Apr. 8, 2024.

(51) Int. Cl. A61M 25/01; A61F 2/24; A61M 25/00

(52) U.S. Cl. CPC A61M 25/0147; A61F 2/2427; A61M 25/0012; A61M 25/0015; A61M 25/0045; A61M 25/0138; A61M 2025/0037; A61M 2025/0095

(57) ABSTRACT

The present disclosure relates to delivery assemblies that include a steerable delivery catheter. In an example, the steerable delivery catheter comprises a slotted tube disposed around a primary lumen thereof, the slotted tube comprising a plurality of slots axially spaced from each other, wherein each slot defines a slot circumferential center between circumferential ends thereof. The steerable delivery catheter further comprises a pull-wire lumen extending along at least a portion of the steerable delivery catheter, through which a pull-wire extends, wherein the pull-wire is attached to the slotted tube. The circumferential center of each slot is circumferentially offset from the circumferential center of an adjacent slot by a helix angle.

### CROSS-REFERENCE TO RELATED APPLICATIONS

This application is a continuation of International Application No. PCT/US2024/038983, filed Jul. 22, 2024, which claims the benefit of U.S. Provisional Application No. 63/528,945, filed Jul. 26, 2023, and U.S. Provisional Application No. 63/631,362, filed Apr. 8, 2024, the contents of which are herein incorporated by reference in their entirety.

### FIELD

The present disclosure relates to catheters and assemblies for delivering prosthetic tools and devices, and to methods and devices for positioning a distal portion of a steerable delivery catheter at a desired site of treatment.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches, such as transcatheter aortic valve replacement (TAVR), are used in various procedures to deliver prosthetic medical devices to locations inside the body that are not readily accessible by surgery or where access without surgery is desirable.

Transcatheter aortic valve replacement (TAVR) is one example of a minimally-invasive surgical procedure used to replace a native aortic valve. In one specific example of the procedure, an expandable prosthetic heart valve is mounted in a crimped state on the distal end of a delivery apparatus and advanced through the patient's vasculature (for example, through a femoral artery and the aorta) to the heart. The prosthetic heart valve is positioned within the native valve and expanded to its functional size.

A variant of TAVR is valve-in-valve (ViV) TAVR, where a new prosthetic heart valve replaces a previously implanted prosthetic valve. In one specific example of the procedure, a new expandable prosthetic heart valve (“guest valve”) is delivered to the heart in a crimped state, as described above for the “native” TAVR. The guest valve is positioned within the previously implanted prosthetic valve (“host valve”) and then expanded to its functional size. The host valve in a ViV TAVR procedure can be a surgically implanted prosthetic valve or a transcatheter prosthetic valve. The term “host valve” is also used herein to refer to the native aortic valve in a native TAVR procedure.

### SUMMARY

One potential technique for mitigating the risk of coronary ostial obstruction involves formation of a hole in one or more leaflets of the host valve (which can be an aortic bioprosthetic valve or a native aortic valve). A guest prosthetic valve can be optionally placed within the leaflet hole and expanded in a manner that tears the host leaflet and prevents it from obstructing the coronary ostium. A flexible delivery catheter can be used to orient an appropriate cutting or lacerating apparatus towards a desired region of treatment, such as toward a left leaflet in vicinity of the left coronary ostium. However, the ability to steer the delivery catheter to a desires position, optionally across the aortic root from one side to the other, can be challenging.

In some aspects of the disclosure, there is provided a delivery assembly comprising a delivery apparatus that includes a steerable delivery catheter. The steerable delivery catheter comprises a primary lumen defining a central longitudinal axis, a slotted tube disposed around the primary lumen along at least a distal segment of the steerable delivery catheter, a pull-wire lumen extending along at least a portion of the steerable delivery catheter, and a pull-wire slidingly extending through the pull-wire lumen and attached to the slotted tube. The slotted tube comprises a plurality of slots axially spaced from each other and a plurality of ribs defined between the slots. Each slot has a slot width and defines a slot circumferential center between circumferential ends of the slot. The circumferential center of each slot is circumferentially offset from the circumferential center of an adjacent slot by a helix angle.

In some examples, the pull-wire is aligned with the slot circumferential centers.

According to one aspect of the disclosure, a delivery assembly comprises a delivery apparatus comprising a steerable deliver catheter.

In some examples, the steerable delivery catheter comprises a primary lumen defining a central longitudinal axis, a slotted tube disposed around the primary lumen along at least a distal segment of the steerable delivery catheter, a pull-wire lumen extending along at least a portion of the steerable delivery catheter, and a pull-wire slidingly extending through the pull-wire lumen and attached to the slotted tube.

In some examples, the slotted tube comprises a plurality of slots axially spaced from each other, wherein each slot has a slot width and defines a slot circumferential center between circumferential ends of the slot and a plurality of ribs defined between the slots.

In some examples, the circumferential center of each slot is circumferentially offset from the circumferential center of an adjacent slot by a helix angle.

In some examples, each slot spans more than 180° of the circumference of the slotted tube.

In some examples, each slot spans more than 220° of the circumference of the slotted tube.

In some examples, each slot spans more than 270° of the circumference of the slotted tube.

In some examples, the helix angle is not greater than 15°.

In some examples, the helix angle is not greater than 10°.

In some examples, the helix angle is not greater than 5°.

In some examples, the pull-wire is aligned with the slot circumferential centers.

In some examples, the pull-wire lumen is aligned with the slot circumferential centers.

In some examples, the slotted tube further comprises a backbone opposite to the slot circumferential centers, defined by uncut portions of the slotted tube between the circumferential ends of the slots.

In some examples, the pull-wire extends opposite to the backbone.

In some examples, the pull-wire lumen extends opposite to the backbone.

In some examples, the slotted tube further comprises a plurality of opposite cuts axially spaced from each other and extending through the backbone.

In some examples, the opposite cuts are axially disposed between the slots.

In some examples, each opposite cut comprises an opening and two slits circumferentially extending therefrom.

In some examples, the openings of the opposite cuts are positioned circumferentially opposite to the slot circumferential centers.

In some examples, the pull-wire extends opposite to the openings of the opposite cuts.

In some examples, the pull-wire lumen extends opposite to the openings of the opposite cuts.

In some examples, the slot width of at least one of the plurality of slots increased in size towards the slot circumferential center.

In some examples, the slot width of at least one of the plurality of slots is uniform between the circumferential ends of the slot.

In some examples, the slots comprise distal slots along a distal portion of the slotted tube, and intermediate slots along an intermediate portion of the slotted tube.

In some examples, the slots further comprise proximal slots along a proximal portion of the slotted tube.

In some examples, the shape of the distal slots is different from the shape of the intermediate slots and/or the proximal slots.

In some examples, the slot width of each slot of the distal slots and the intermediate slots increases towards the corresponding slot circumferential center.

In some examples, the slot width of each slot of the proximal slots is uniform between the corresponding circumferential ends.

In some examples, the shape of the distal slots is different from the shape of at least one of the intermediate slots or the proximal slots.

In some examples, each slot defines a slot length between its circumferential ends, and wherein the slot length of the distal slots is different from the slot length of the intermediate slots and/or the proximal slots.

In some examples, the width of the ribs disposed between the distal slots is different from the width of the ribs disposed between the intermediate slots and/or the proximal slots.

In some examples, the ribs between the distal slots are narrower than the ribs between the intermediate slots.

In some examples, the ribs between the intermediate slots are narrower than the ribs between the proximal slots.

In some examples, the helix angle comprises a distal helix angle by which the distal slots are angularly offset from each other, an intermediate helix angle by which the intermediate slots are angularly offset from each other, and a proximal helix angle by which the distal slots are angularly offset from each other.

In some examples, the distal helix angle is different from at least one of the intermediate helix angle and/or the proximal helix angle.

In some examples, a distal-most slot of the intermediate slots is angularly offset from a proximal-most slot of the distal slots by an angle between the distal helix angle and the intermediate helix angle, inclusive.

In some examples, a distal-most slot of the proximal slots is angularly offset from a proximal-most slot of the intermediate slots by an angle between the intermediate helix angle and the proximal helix angle, inclusive.

In some examples, the slots are orthogonal to an angled axis, defined as an axis which is angled at the helix angle relative to the central longitudinal axis.

In some examples, the pull-wire is coupled to an actuator of a handle of the delivery apparatus, wherein the actuator is configured to apply tension to the pull-wire.

In some examples, the actuator comprises a rotatable knob.

In some examples, tension applied to the pull-wire is configured to bend the distal portion of the steerable delivery catheter in a three-dimensional out-of-plane manner.

In some examples, the steerable delivery catheter further comprises a polymeric layer disposed around the primary lumen.

In some examples, the slotted tube is embedded within the polymeric layer.

In some examples, the pull-wire lumen is embedded within the polymeric layer.

In some examples, the slotted tube is disposed radially outward from the pull-wire lumen along at least a portion of the pull-wire lumen.

In some examples, the steerable delivery catheter further comprises a braid.

In some examples, the braid is embedded within the polymeric layer.

In some examples, the braid is disposed between the pull-wire lumen and the slotted tube.

In some examples, the polymeric layer comprises an encapsulating polymeric layer, and an outer polymeric layer disposed around the encapsulating polymeric layer.

In some examples, the encapsulating polymeric layer has a non-uniform cross-sectional thickness, and wherein the pull-wire lumen extends through a thicker wall section of the encapsulating polymeric layer.

In some examples, the steerable delivery catheter further comprises a pull ring, and wherein the pull-wire is coupled to the pull-wing.

In some examples, the pull ring is embedded within the polymeric layer.

In some examples, the pull ring is coupled to the slotted tube.

In some examples, the pull ring comprises at least one cut-out, and wherein the slotted tube further comprises at least one tube teeth received within the at least one cut-out.

In some examples, the pull ring is integrally formed with the slotted tube.

In some examples, the pull ring comprises one or more windows through which the polymeric layer radially extends.

In some examples, the pull ring comprises a wire groove through which the pull-wire extends.

In some examples, the slotted tube comprises a semi-circular cut-out aligned with the wire groove.

In some examples, the polymeric layer comprises a polymeric material selected from at least one of: polyamides and polyether block amides.

In some examples, the polymeric layer comprises a first polymer having a first stiffness along the distal segment of the steerable delivery catheter, a second polymer having a second stiffness along an intermediate segment of the steerable delivery catheter, and a third polymer having a third stiffness along a proximal segment of the steerable delivery catheter.

In some examples, the first stiffness is less than the second stiffness.

In some examples, the second stiffness is less than the third stiffness.

The term “stiffness” is as defined below. It is to be understood that in the context of first and second stiffnesses, that, while the compositions of the polymeric layer components may have temperature-related stiffness, the relative values stiffness are determined at room temperature. Thus, in some examples, the phrase “the first stiffness is less than the second stiffness” means that at least at room temperature the first stiffness is less than the second stiffness.

In some examples, the steerable delivery catheter further comprises a primary lumen liner around the primary lumen, wherein the primary lumen liner is radially internal to the polymeric layer.

In some examples, the primary lumen liner comprises polytetrafluoroethylene.

In some examples, the steerable delivery catheter further comprises a pull-wire lumen liner around the pull-wire lumen.

In some examples, the pull-wire lumen liner comprises polytetrafluoroethylene.

In some examples, the steerable delivery catheter further comprises a tip extending between the slotted tube and a catheter distal end.

In some examples, the distal non-steerable portion has a length which is greater than a diameter defined by the primary lumen.

In some examples, the delivery apparatus further comprises an inner catheter axially movable through the primary lumen.

In some examples, the delivery apparatus further comprises a perforating device that comprises a perforating member disposed within the inner catheter, and an expansion member supported by a distal end portion of the inner catheter.

In some examples, the perforating member is configured to pierce a host leaflet of a host valvular structure to form a pilot puncture in the host leaflet.

In some examples, the expansion member is configured to be inserted within the pilot puncture and to be selectively transitioned between a radially compressed configuration and a radially expanded configuration.

In some examples, transitioning the expansion member to the radially expanded configuration, while positioned within the pilot puncture, is configured to expand the pilot puncture to form a leaflet opening.

In some examples, the perforating member comprises a distal end portion configured to be positioned distal to the expansion member for formation of the pilot puncture.

In some examples, the distal end portion of the perforating member is axially movable relative to the expansion member.

In some examples, the distal end portion of the perforating member terminates at an angled surface.

In some examples, the perforating member comprises a needle.

In some examples, the needle is one or both of a spring-loaded needle and a Veress needle.

In some examples, the delivery assembly further comprises a guidewire extending through the perforating member.

In some examples, the guidewire comprises a sharp tip configured to penetrate through the host leaflet.

In some examples, the delivery assembly further comprises an RF energy source coupled to the guidewire and configured to provide RF energy to a tip of the guidewire.

In some examples, the perforating member is a guidewire extending through the inner catheter.

In some examples, the delivery assembly further comprises an RF energy source coupled to the guidewire and configured to provide RF energy to a tip of the guidewire.

In some examples, the guidewire comprises a sharp tip configured to penetrate through the host leaflet.

In some examples, the expansion member is an inflatable balloon, and wherein the inner catheter is a balloon catheter.

In some examples, the delivery apparatus further comprises a dilator shaft extending through the inner catheter, and a dilator attached to the dilator shaft.

In some examples, the dilator is distal to the expansion member.

In some examples, the perforating member is axially movable relative to the dilator shaft.

In some examples, the dilator is a nosecone, and wherein the dilator shaft is a nosecone shaft.

In some examples, the host valvular structure is a native valvular structure of native heart valve.

In some examples, the host valvular structure is a valvular structure of previously implanted prosthetic valve that is implanted within a native heart valve.

In some examples, the delivery assembly further comprises a prosthetic valve comprising a frame movable between a radially compressed and a radially expanded configuration.

In some examples, the slot width of at least some of the slots gradually changes along a length of the slotted tube.

In some examples, the slot width of at least some subsequent slots of the plurality of slots gradually increases in the distal direction.

In some examples, one or more of the ribs comprises a protrusion extending towards a complementary recess formed in an adjacent one of the ribs.

In some examples, the protrusion is positioned at the slot circumferential center of the corresponding slot.

In some aspects of the disclosure, there is provided a method for modifying a steerable delivery catheter, the method comprising: providing a delivery catheter, which comprises: a primary lumen defining a central longitudinal axis; a polymeric layer disposed around the primary lumen; a slotted tube embedded in the polymeric layer and disposed around the primary lumen along at least a distal segment of the steerable delivery catheter, the slotted tube comprising a plurality of slots axially spaced from each other, wherein each slot defines a slot circumferential center between circumferential ends of the slot, wherein a distal-most slot is circumferentially aligned with an adjacent slot along the length of the slotted tube; holding the polymeric layer at a first holding point and at a second holding point, wherein the first holding point is axially spaced from the second holding point, wherein the second holding point is in mechanical communication with a distal end portion of the slotted tube and the first holding point is in mechanical communication with a proximal end and/or an intermediate portion of the slotted tube; imparting energy to a portion of the polymeric layer, which is in mechanical communication with the slotted tube, thereby increasing the malleability of the portion of the polymeric layer; and rotating one of the first and second holding points around the central longitudinal axis relative to the other holding point, thereby twisting the polymeric layer and slotted tube radially around the central longitudinal axis, so that the circumferential center of the distal-most slot is circumferentially offset from the circumferential center of the adjacent slot by a helix angle.

In some examples, at least 25% of the slots of the slotted tube of the provided delivery catheter are circumferentially aligned with each other along the length of the slotted tube.

In some examples, each one of the slots of the slotted tube of the provided delivery catheter are circumferentially aligned with each other along the length of the slotted tube. In some examples, the helix angle is not greater than 15°.

In some examples, upon the rotation the pull-wire is aligned with the slot circumferential centers.

In some examples, upon the rotation the pull-wire lumen is aligned with the slot circumferential centers.

In some examples, the polymeric layer has a temperature-dependent malleability.

In some examples, the polymeric layer comprises at least one polymer having a glass transition temperature in the range of 50° C. to 250° C.

In some examples, the method comprises holding the polymeric layer only at the first holding point and at the second holding point.

In some examples, at least part of a length of the slotted tube is axially disposed between the first holding point and the second holding point.

In some examples, at least 50% of the length of the slotted tube is axially disposed between the first holding point and the second holding point.

In some examples, wherein the entire length of the slotted tube is axially disposed between the first holding point and the second holding point.

In some examples, an axial distance between the first holding point and the second holding point is at least 500% greater than an axial distance between the second holding point and the distal end portion of the slotted tube.

In some examples, wherein the first holding point is in mechanical communication with the proximal end portion of the slotted tube.

In some examples, the proximal end portion of the slotted tube is embedded within the polymeric layer proximally to the first holding point.

In some examples, the method further comprises preventing from the delivery catheter to substantially collapse inwards upon the holding at the first holding point.

In some examples, the method further comprises maintaining at least one rod within the primary lumen.

In some examples, at least one rod has a first holding point, wherein the method comprises maintaining the rod first holding point laterally substantially in parallel to the polymeric layer first holding point.

In some examples, holding the polymeric layer at the first holding point includes applying mechanical pressure on the polymeric layer first holding point radially inward, thereby also applying mechanical pressure on the rod first holding point radially inward.

In some examples, at least one rod has a second holding point, wherein the method comprises maintaining the rod second holding point laterally substantially in parallel to the polymeric layer first holding point.

In some examples, holding the polymeric layer at the second holding point includes applying mechanical pressure on the polymeric layer second holding point radially inward, thereby also applying mechanical pressure on the rod second holding point radially inward.

In some examples, the method comprises maintaining a first rod and a second rod within the primary lumen.

In some examples, method comprises maintaining a first rod and a second rod substantially axially aligned within the primary lumen.

In some examples, the first rod has a proximal end portion and a distal end portion, wherein the second rod has a proximal end portion and a distal end portion, and wherein the method comprises maintaining the first rod distal end portion adjacent to the second rod proximal end portion.

In some examples, the first rod distal end portion is in the form of a protrusion and the second rod proximal end portion is in the form of a recess, or wherein the first rod distal end portion is in the form of a recess and the second rod proximal end portion is in the form of a protrusion, and wherein the protrusion is inserted within the recess to maintain substantial axial alignment between the first rod and the second rod.

In some examples, imparting energy to the portion of the polymeric layer includes elevating the temperature of the portion of the polymeric layer.

In some examples, elevating the temperature of the portion of the polymeric layer includes applying external heat to the portion of the polymeric layer using a heating device. In some examples, the heating device comprises a heat blower.

In some examples, imparting energy to the portion of the polymeric layer includes adjusting the temperature of the portion of the polymeric layer to a temperature T^{c}.

In some examples, temperature T^{c} is equal or above the glass transition temperature of at least one polymer comprised within the polymeric layer.

In some examples, temperature T^{c} is equal or above the glass transition temperature of the polymeric layer.

In some examples, temperature T^{c} is at least 50° C.

In some examples, the portion of the polymeric layer to which energy is imparted to is located between the first holding point and the second holding point of the polymeric layer.

In some examples, upon the imparting of the energy to the portion of the polymeric layer, a temperature T^{c} of the portion of the polymeric layer is higher than the temperature of the first holding point.

In some examples, upon the imparting of the energy to the portion of the polymeric layer, the portion of the polymeric layer becomes at least partially malleable.

In some examples, imparting of the energy to the portion of the polymeric layer includes increasing the malleability of the portion of the polymeric layer.

In some examples, rotating one of the first and second holding points around the central longitudinal axis relative to the other holding point includes rotating one of the first and second holding points around the central longitudinal axis and holding the other holding point static.

In some examples, the method comprises rotating the second holding point around the central longitudinal axis and holding the first holding point static.

In some examples, the method comprises synchronically twisting the polymeric layer and slotted tube radially.

In some examples, the provided delivery catheter comprises a braid, which has a portion extending axially at least between the first holding point and the second holding point of the polymeric layer.

In some examples, the method comprises twisting the braid portion radially around the central longitudinal axis.

In some examples, the method comprises synchronically twisting the braid portion and the polymeric layer radially around the central longitudinal axis.

In some examples, the method comprises synchronically twisting the braid portion and the slotted tube radially around the central longitudinal axis.

In some examples, the method comprises providing a twisting apparatus and holding the polymeric layer at a first holding point and at a second holding point; and rotating one of the first and second holding points around the central longitudinal axis relative to the other holding point using the twisting apparatus.

In some examples, the method comprises a step of lowering the temperature of the portion of the polymeric layer.

In some examples, lowering the temperature of the portion of the polymeric layer includes adjusting the temperature of the portion of the polymeric layer to a temperature T^{e}.

In some examples, temperature T^{e} is equal or below the glass transition temperature of at least one polymer comprised within the polymeric layer.

In some examples, the portion of the polymeric layer of which the temperature is lowered is located between the first holding point and the second holding point of the polymeric layer.

In some examples, lowering the temperature of the portion of the polymeric layer includes reducing the malleability of the portion of the polymeric layer.

In some examples, the method comprises maintaining the portion of the polymeric layer at ambient temperature for a time period sufficient reduce the relative elongation of the polymeric layer by at least 5%.

In some aspects of the disclosure, there is provided a delivery apparatus comprising a steerable delivery catheter which comprises a primary lumen defining a central longitudinal axis, and a slotted tube disposed around the primary lumen along at least a distal segment of the steerable delivery catheter.

In some examples, the slotted tube comprises a plurality of slots.

In some examples, a circumferential center of each slot is circumferentially offset from a circumferential center of an adjacent slot by a helix angle.

In some examples, each of the plurality of slots has a slot width and defines the slot circumferential center between circumferential ends of the slot.

In some examples, the slotted tube comprises a plurality of ribs defined between the slots.

In some examples, the steerable delivery catheter comprises a pull-wire lumen extending along at least a portion of the steerable delivery catheter.

In some examples, the steerable delivery catheter comprises a pull-wire slidingly extending through the pull-wire lumen and attached to the slotted tube.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, which proceeds with reference to the accompanying figures.

### BRIEF DESCRIPTION OF THE FIGURES

Some examples of the disclosure are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the disclosure. For the sake of clarity, some objects depicted in the figures are not to scale.

In the Figures:

FIG. 1 is a sectional view of an aortic root.

FIG. 2A shows a cross-sectional view of a prosthetic heart valve implanted in the native aortic valve of within the aortic root of FIG. 1, according to an example.

FIG. 2B shows the implanted prosthetic heart valve of FIG. 1A as viewed from the ascending aorta, according to an example.

FIG. 3 shows a valve-in-valve implantation within the native aortic valve of FIG. 1, according to an example.

FIG. 4 shows an exemplary delivery assembly comprising a steerable delivery catheter.

FIG. 5 shows a sectional view of a delivery assembly being delivered in situ to a treatment site.

FIG. 6 shows a distal segment of an exemplary steerable delivery catheter, with some components and/or layers thereof removed from view.

FIG. 7A shows a perspective view of a distal segment of an exemplary steerable delivery catheter.

FIG. 7B shows a longitudinal sectional view of the distal segment of the steerable delivery catheter of FIG. 7B.

FIG. 7C a lateral cross-sectional view along line 7C-7C of FIG. 7A.

FIG. 8A shows a side view of an exemplary slotted tube, configured for planar articulation.

FIG. 8B shows the slotted tube of FIG. 8A in a flattened configuration.

FIG. 8C shows a side view of a distal region of the slotted tube of FIG. 8A.

FIG. 8D shows a top view of the distal region of the slotted tube of FIG. 8C.

FIG. 8E shows a bottom view of the distal region of the slotted tube of FIG. 8C.

FIG. 8F shows a flattened configuration of the distal region of the slotted tube of FIG. 8C.

FIG. 9A shows an exemplary steerable delivery catheter advanced, along the aortic arch, towards the native aortic valve.

FIG. 9B shows the distal portion of the steerable delivery catheter of FIG. 9A deflected partially sideways.

FIG. 10A shows a side view of an exemplary slotted tube, configured for out-of-plane articulation.

FIG. 10B shows the slotted tube of FIG. 10A in a flattened configuration.

FIG. 10C shows a side view of a distal region of the slotted tube of FIG. 10A.

FIG. 10D shows a top view of the distal region of the slotted tube of FIG. 10C.

FIG. 10E shows a bottom view of the distal region of the slotted tube of FIG. 10C.

FIG. 10F shows a flattened configuration of the distal region of the slotted tube of FIG. 10C.

FIG. 11A shows a top view of a distal segment of an exemplary steerable delivery catheter, configured for out-of-plane articulation.

FIG. 11B shows a perspective side view of the distal segment of the steerable delivery catheter of FIG. 11A.

FIG. 12A shows an exemplary steerable delivery catheter advanced, along the aortic arch, towards the native aortic valve.

FIG. 12B shows the distal segment of the steerable delivery catheter of FIG. 12A deflected to an opposite side of the aortic valve relative to the position of FIG. 12A.

FIG. 13A shows a side view of an exemplary slotted tube, devoid of opposite cuts.

FIG. 13B shows a flattened view of the slotted tube of FIG. 13A.

FIG. 14A shows a side view of a distal region of an exemplary slotted tube, having slots orthogonal to an angled axis thereof.

FIG. 14B shows a flattened view of the slotted tube of FIG. 14A.

FIG. 15 shows a flattened view of an exemplary slotted tube, having different helix angles along different portions thereof.

FIG. 16 shows a top view of a distal segment of an exemplary steerable delivery catheter, having an elongated distal non-steerable portion.

FIG. 17A is a simplified side view of a delivery apparatus with a perforating device extendable through a steerable delivery catheter, positioned on an outflow side of a host leaflet according to an example.

FIG. 17B is a simplified side view of the delivery apparatus of FIG. 9A with a needle piercing the host leaflet.

FIG. 17C is a simplified side view of the delivery apparatus of FIG. 9A with an inflatable balloon positioned within the host leaflet in a deflated state.

FIG. 17D is a simplified side view of the delivery apparatus of FIG. 9A with the balloon positioned within the host leaflet in an inflated state.

FIG. 18A shows the delivery apparatus of FIG. 17A with the balloon positioned within a pilot puncture of the host leaflet in a deflated state.

FIG. 18B shows the balloon of FIG. 18A inflated within the host leaflet.

FIG. 19 shows a previously implanted prosthetic valve subsequent to forming the leaflet opening in a host leaflet thereof.

FIG. 20 shows a configuration in which a guest prosthetic valve has been expanded within the leaflet opening of a host prosthetic valve.

FIG. 21 shows a perspective view of an exemplary slotted tube having ribs equipped with protrusions.

FIG. 22 shows a top view of an exemplary slotted tube having gradually varying widths between sequential slots.

FIG. 23 shows a simplified view of a distal portion of a slotted tube that includes two slots which are circumferentially offset from each other.

FIG. 24 shows a simplified sectional view of the slotted tube of FIG. 23 along the radial plane.

FIG. 25 shows a simplified flattened view of the slotted tube of FIG. 23.

FIG. 26A shows a side view of a slotted tube curved in a two-dimensional plane.

FIG. 26B shows a top view of the curve slotted tube of FIG. 26A.

FIGS. 27A and 27B are top views of examples of a slotted tube curved in a three-dimensional/out-of-plane configuration.

FIG. 28 is a perspective view of an exemplary delivery apparatus that can be used for modifying a steerable delivery catheter.

FIG. 29 is a sectional side view of the delivery apparatus of FIG. 28.

FIGS. 30A-30C are enlarged views of selected regions of FIG. 29.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a”, “an”, and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner”, “outer”, “upper”, “lower”, “inside”, “outside”, “top”, “bottom”, “interior”, “exterior”, “left”, right”, and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (for example, inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (for example, the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

The terms “axial direction”, “radial direction”, and “circumferential direction” have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used herein, terms such as “first”, “second”, and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “at least substantially parallel” refers to directions that are fully parallel, and to directions that diverge by up to 22.5 degrees.

In the present disclosure, a reference numeral that includes an alphabetic label (for example, “a”, “b”, “c”, etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

Described herein are steerable delivery catheters and related methods, which can be used to deliver tools and prosthetic devices to a location within a body of a subject. In some examples, steerable delivery catheters described herein can be used to deliver tools for modifying leaflets of an existing valvular structure in a patient's heart, and/or for implanting prosthetic valves. Prior to or during implantation of the prosthetic heart valve within the existing valvular structure, each device, such as a delivery apparatus that can optionally carry a prosthetic valve, can be provided in the ascending aorta of a patient and can be used to pierce, lacerate, slice, tear, cut or otherwise modify a leaflet or commissure of the existing valvular structure. In some examples, the existing valvular structure can be a native aortic valve (for example, normal or abnormal, such as bicuspid aortic valve (BAV)) or a prosthetic valve previously implanted in the native aortic valve.

The modification can avoid, or at least reduce the likelihood of, issues that leaflets of the existing valvular structure might otherwise cause once the prosthetic heart valve has been fully installed, for example, obstruction of blood flow to the coronary arteries, improper mounting due to a non-circular valve cross-section, and/or restricted access to the coronary arteries if subsequent intervention is required. While described with respect to aortic valve, it should be understood that the disclosed examples can be adapted to deliver devices, such as cutting tools and/or implantable prosthetic devices, to and/or in any of the native annuluses of the heart (for example, the aortic, pulmonary, mitral, and tricuspid annuluses), and can be used with any of various delivery approaches (for example, retrograde, antegrade, transseptal, transventricular, transatrial, etc.).

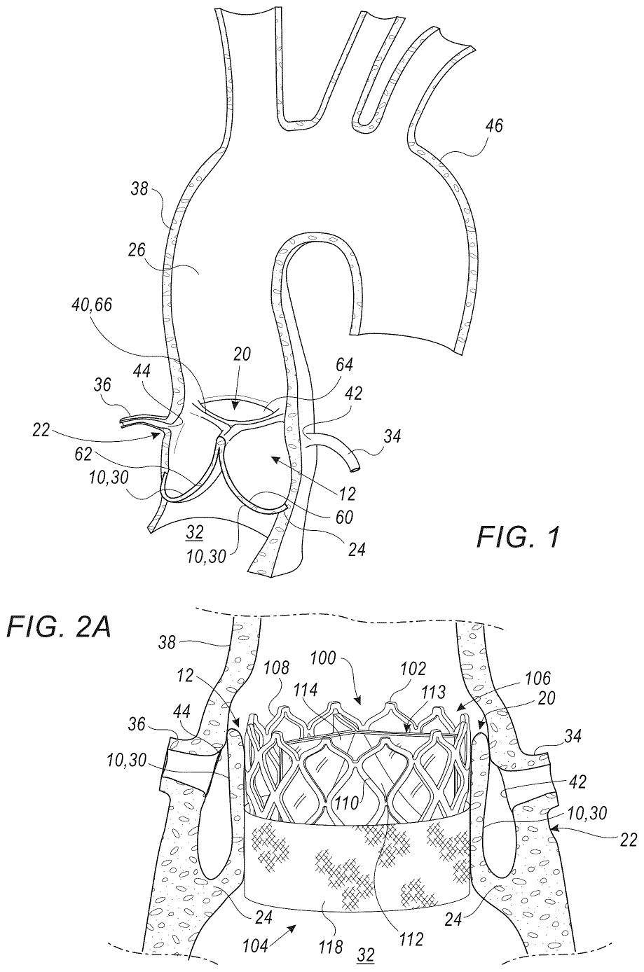

FIG. 1 illustrates an anatomy of the aortic root 22, which is positioned between the left ventricle 32 and the ascending aorta 26. The aortic root 22 includes a native aortic valve 20 having a native valvular structure 29 comprising a plurality of native leaflets 30. Normally, the native aortic valve 20 has three leaflets, but aortic valves with fewer than three leaflets are possible. The three native leaflet 30 of the aortic valve 20 include the left leaflet 60, right leaflet 62, and non-coronary leaflet 64. The leaflets 30 are supported at native commissures 40 by the aortic annulus 24, which is a ring of fibrous tissue at the transition point between the left ventricle 32 and the aortic root 22. The leaflets 30 can cycle between open and closed positions (the closed position is shown in FIG. 1) to regulate flow of blood from the left ventricle 32 to the ascending aorta 26. Branching off the aortic root 22 are the left coronary artery 34 and the right coronary artery 36. The coronary ostia 42, 44 are the openings that connect the aortic root 22 to the coronary arteries 34, 36. The left coronary ostium 42 is positioned proximate to the left leaflet 60, and the right coronary ostium 44 is positioned proximate to the right leaflet 62.

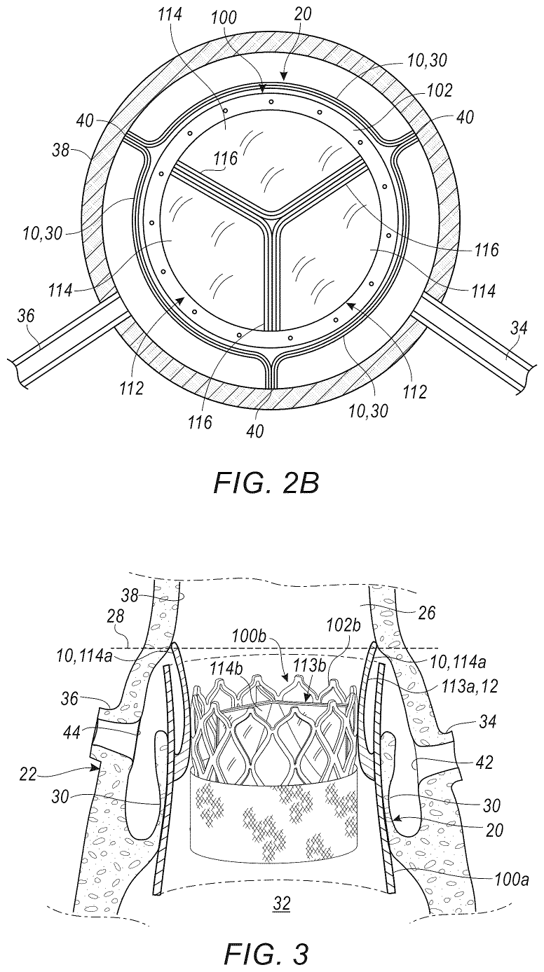

FIGS. 2A-2B show an exemplary prosthetic valve 100 that can be implanted in a native heart valve, such as the native aortic valve 20 of FIG. 1. The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valve can be crimped on or retained by an implant delivery apparatus in the radially compressed state during delivery, and then expanded to the radially expanded state once the prosthetic valve reaches the implantation site. The expanded state may include a range of diameters to which the valve may expand, between the compressed state and a maximum diameter reached at a fully expanded state. Thus, a plurality of partially expanded states may relate to any expansion diameter between radially compressed or crimped state, and maximum expanded state. A prosthetic valve of the current disclosure (for example, prosthetic valve 100) may include any prosthetic valve configured to be mounted within the native aortic valve, the native mitral valve, the native pulmonary valve, and the native tricuspid valve.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (not shown). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US2021/052745 and U.S. Provisional Application Nos. 63/85,947 and 63/209904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

FIGS. 2A-2B show an example of a prosthetic valve 100, which can be a balloon expandable valve or any other type of valve, illustrated in an expanded state. The prosthetic valve 100 can comprise an outflow end 106 and an inflow end 104. In some instances, the outflow end 106 is the proximal end of the prosthetic valve 100, and the inflow end 104 is the distal end of the prosthetic valve 100. Alternatively, depending for example on the delivery approach of the valve, the outflow end can be the distal end of the prosthetic valve, and the inflow end can be the proximal end of the prosthetic valve.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The terms “longitudinal” and “axial”, as used herein, refer to an axis extending in the proximal and distal directions, unless otherwise expressly defined.

The prosthetic valve 100 comprises an annular frame 102 movable between a radially compressed configuration and a radially expanded configuration, and a valvular structure 113 that comprises prosthetic valve leaflets 114 mounted within the frame 102. The frame 102 can be made of various suitable materials, including plastically-deformable materials such as, but not limited to, stainless steel, a nickel-based alloy (for example, a nickel-cobalt-chromium alloy such as MP35N alloy), polymers, or combinations thereof. When constructed of a plastically-deformable materials, the frame 102 can be crimped to a radially compressed state on a balloon catheter, and then expanded inside a patient by an inflatable balloon or equivalent expansion mechanism. Alternatively or additionally, the frame 102 can be made of shape-memory materials such as, but not limited to, nickel titanium alloy (for example, Nitinol). When constructed of a shape-memory material, the frame 102 can be crimped to a radially compressed state and restrained in the compressed state by insertion into a shaft or equivalent mechanism of a delivery apparatus.

In the example illustrated in FIGS. 2A-2B, the frame 102 is an annular, stent-like structure comprising a plurality of intersecting struts 108. In this application, the term “strut” encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 108 may be any elongated member or portion of the frame 102. The frame 102 can include a plurality of strut rungs that can collectively define one or more rows of cells 110. The frame 102 can have a cylindrical or substantially cylindrical shape having a constant diameter from the inflow end 104 to the outflow end 106 as shown, or the frame can vary in diameter along the height of the frame, as disclosed in U.S. Pat. No. 9,155,619, which is incorporated herein by reference.

The struts 108 can include a plurality of angled struts and vertical or axial struts. At least some of the struts 108 can be pivotable or bendable relative to each other, so as to permit frame expansion or compression. For example, the frame 102 can include a single piece of material, such as a metal tube, via various processes such as, but not limited to, laser cutting, electroforming, and/or physical vapor deposition, while retaining the ability to collapse/expand radially in the absence of hinges and like.

A valvular structure 113 of the prosthetic valve 100 can include a plurality of prosthetic valve leaflets 114 (for example, three leaflets), positioned at least partially within the frame 102, and configured to regulate flow of blood through the prosthetic valve 100 from the inflow end 104 to the outflow end 106. While three leaflets 114 arranged to collapse in a tricuspid arrangement, are shown in the example illustrated in FIGS. 2A-2B, it will be clear that a prosthetic valve 100 can include any other number of leaflets 114. Adjacent leaflets 114 can be arranged together to form prosthetic valve commissures 116 that are coupled (directly or indirectly) to respective portions of the frame 102, thereby securing at least a portion of the valvular structure 113 to the frame 102. The prosthetic valve leaflets 114 can be made from, in whole or part, biological material (for example, pericardium), bio-compatible synthetic materials, or other such materials. Further details regarding transcatheter prosthetic valves, including the manner in which leaflets 114 can be coupled to the frame 102 of the prosthetic valve 100, can be found, for example, in U.S. Pat. Nos. 6,730,118, 7,393,360, 7,510,575, 7,993,394, 8,652,202, and 11,135,056, all of which are incorporated herein by reference in their entireties.

In some examples, the prosthetic valve 100 can comprise at least one skirt or sealing member. For example, the prosthetic valve 100 can include an inner skirt (not shown in FIG. 2A-2B), which can be secured to the inner surface of the frame 102. Such an inner skirt can be configured to function, for example, as a sealing member to prevent or decrease perivalvular leakage. An inner skirt can further function as an anchoring region for leaflets 114 to the frame 102, and/or function to protect the leaflets 114 against damage which may be caused by contact with the frame 102, for example during valve crimping or during working cycles of the prosthetic valve 100. An inner skirt can be disposed around and attached to the inner surface of frame 102, while the leaflets can be sutured to the inner skirt along a scalloped line (not shown). An inner skirt can be coupled to the frame 102 via sutures or another form of coupler.

The prosthetic valve 100 can comprise, in some examples, an outer skirt 118 mounted on the outer surface of frame 102 (as shown in FIGS. 2A-2B), configured to function, for example, as a sealing member retained between the frame 102 and the surrounding tissue of the native annulus against which the prosthetic valve is mounted, or against an inner side of a previously implanted valve in the case of ViV procedures (described further below), thereby reducing risk of paravalvular leakage (PVL) past the prosthetic valve 100. The outer skirt 118 can be coupled to the frame 102 via sutures or another form of coupler.

Any of the inner skirt and/or outer skirt can be made of various suitable biocompatible materials, such as, but not limited to, various synthetic materials (for example, PET) or natural tissue (for example pericardial tissue). In some cases, the inner skirt can be formed of a single sheet of material that extends continuously around the inner surface of frame 102. In some cases, the outer skirt 118 can be formed of a single sheet of material that extends continuously around the outer surface of frame 102.

The cells 110, defined by interconnected struts 108, define cell openings 112. While some of the cell openings 112 can be covered by the inner skirt and/or the outer skirt, at least a portion of the cell opening 112 can remain uncovered, such as cell openings 112 which are closer to the outflow end 106 of the prosthetic valve.

FIGS. 2A-2B illustrate a hypothetical coronary artery obstruction that could occur in some cases from implantation of a prosthetic valve 100 within the native aortic valve 20. In this example, the prosthetic valve 100 is the guest valve or new valve, and the native aortic valve 20 is the host valve or old valve.

During implantation of the prosthetic valve 100, the prosthetic valve 100 is positioned within a central region defined between the native leaflets 30, which are also the host leaflets 10 for the example illustrated in FIG. 2A-2B. The prosthetic valve 100 is then radially expanded against the host leaflets 10. As illustrated, the host leaflets 10 form a tube around the frame 102 of the prosthetic valve 100 upon the prosthetic valve 100 is radially expanded to the working diameter. As further illustrated, expansion of the prosthetic valve 100 displaces the host leaflets 10 outwards towards the coronary ostia 42, 44 such that the host leaflets 10 contact a portion of the aortic root 22 surrounding the coronary ostia 42, 44, causing coronary artery obstruction.

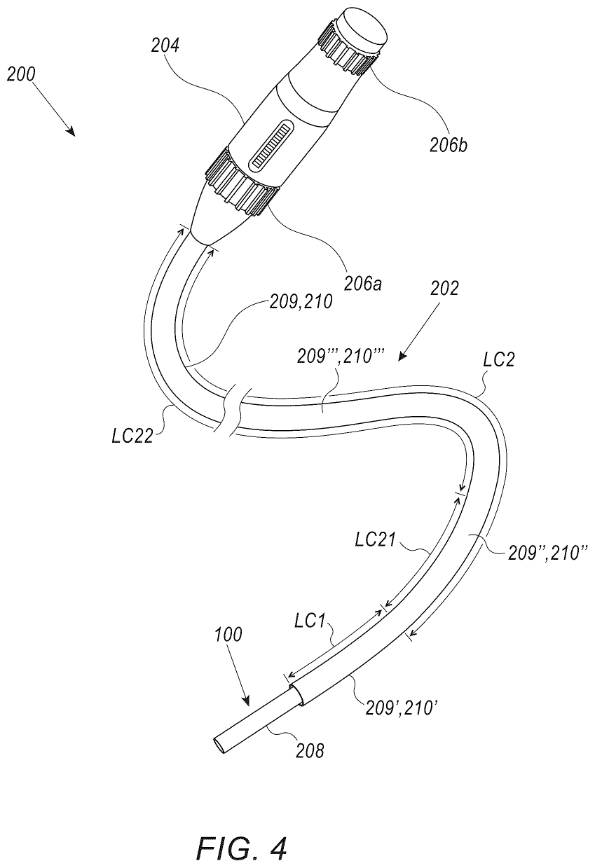

For an existing implanted prosthetic valve, the valvular structure may naturally degrade over time thereby requiring repair or replacement in order to maintain adequate heart functions. In a Valve-in-Valve (ViV) procedure, a new prosthetic heart valve is mounted within the existing, degrading prosthetic heart valve in order to restore proper function. FIG. 3 illustrates an exemplary hypothetical coronary artery obstruction that could occur in some cases from implantation of a prosthetic valve 100b within a previously implanted prosthetic valve 100a (for example, after a ViV procedure). In this example, the prosthetic valve 100b is the guest valve or new valve, and the prosthetic valve 100a is the host valve or old valve. In this example, the prosthetic valve 100a was previously implanted within the orifice of the native aortic valve 20. Each of the prosthetic valves 100a, 100b can have the general structure of the prosthetic valve 100 described with reference to FIGS. 2A-2B, though in some examples, each of the prosthetic valves 100a, 100b can be a different type of prosthetic valve. For example, a balloon expandable guest valve 100b can be implanted inside a previously implanted mechanically expandable or self-expandable host valve 100a.

During implantation of the prosthetic valve 100b, the prosthetic valve 100b is positioned within a central region defined between the leaflets 114a of the prosthetic valve 100a, which now take the role of host leaflet 10. The prosthetic valve 100b is then radially expanded against the host leaflets 10 (i.e., against the prosthetic valve leaflets 114c). As illustrated, the radial expansion of the prosthetic valve 100a results in outward displacement of the host leaflets 10. As further illustrated, the host leaflets 10 are displaced such that the host leaflets 10 contact the aortic root 22 at positions superior to the coronary ostia 42, 44, causing coronary artery ostia obstruction. Alternatively, the guest prosthetic valve 100b can displace the host leaflets 114a outwardly against the frame 102a of the host valve 100a, thereby blocking the flow of blood through the frame 102a to the coronary ostia 42, 44.

In some patient anatomies (for example, when the outflow end 106 of the prosthetic valve 100 is at the STJ level 28 and the diameter of the prosthetic valve 100 is similar to the STJ diameter such that the frame 102 touches or is very close to the aortic wall 38 at the STJ level 28), the host leaflets 10 may compromise the ability for future access into the coronary arteries 34, 36 or perfusion through the frame 102 to the coronary arteries 34, 36 during the diastole phase of the cardiac cycle. Similar problems may occur in some patient anatomies either when a guest prosthetic valve 100b is percutaneously expanded within a previously implanted host prosthetic valve 100a, or when a prosthetic valve 100 is percutaneously expanded within a native valve, displacing the native leaflets 30 outward toward the coronary ostia 42, 44.

The risk illustrated in FIG. 3 may be higher when the host valve is a bioprosthetic valve without a frame or when the leaflets of the host valve are external to a frame. Risk of coronary artery ostia obstruction can increase in a cramped aortic root or when the coronary artery ostium sits low. In the examples illustrated in FIGS. 2A-3, the host leaflets 10 are shown obstructing both coronary ostia 42, 44. In some cases, only one host leaflet 10 may obstruct a respective coronary artery ostium. For example, the risk of obstructing the left coronary ostium 42 tends to be greater than obstructing the right coronary ostium 44 because the left coronary ostium 42 can sit lower than the right coronary ostium 44.

The term “host valve” as used herein refers to a native heart valve in which a prosthetic valve is implanted or a previously implanted prosthetic valve in which a new prosthetic valve is implanted. Moreover, in any of the examples disclosed herein, when the host valve is a previously implanted prosthetic valve, the host valve can be a surgically implanted prosthetic heart valve (known as a “surgical valve”) or a transcatheter heart valve. The term “guest valve”, as used herein, refers to a prosthetic valve implanted in a host valve, which can be either a native heart valve or a previously implanted prosthetic valve. Similarly, the term “host leaflets 10”, as used herein, refers to native leaflets 30 of a native valve in which a new guest prosthetic valve 100 is implanted, or to prosthetic valve leaflets 114a of a previously implanted host valve 100a in which a new guest prosthetic valve 100b is implanted.

To avoid obstruction of blood flow to the coronary arteries 34, 36, the valvular structure 12 of the existing host valve (whether a native aortic valve or a previously implanted prosthetic valve) can be modified by components of a delivery apparatus prior to or during implantation of a new prosthetic valve within the existing valvular structure 12. In some examples, the host valvular structure 12 is modified by piercing, lacerating, tearing, slicing, and/or cutting one or more host leaflets 10 (for example, a free end of the host leaflet 10 or a commissure of adjacent host leaflets 10, which can be a native commissure 40 for a native aortic valve 20, or a prosthetic valve commissure 116 for a previously implanted host prosthetic valve 100) using the delivery apparatus. The modification thus disrupts the impermeable tubular structure that would otherwise be formed by the existing host leaflets 10, thereby allowing blood to flow to the coronary arteries 34, 36. In some examples, a delivery apparatus according to any example described throughout the current disclosure, can be configured to deliver a device configured to modify the host valvular structure 12 (i.e., modify at least one of the host leaflets 10). In some examples, a delivery apparatus according to any example described throughout the current disclosure, can be configured to deliver and implant a guest prosthetic valve 100 within a modified valvular structure 12.

A delivery assembly comprising any delivery apparatus described throughout the current disclosure can be utilized, for example, to deliver a cutting tool for modifying a valvular structure at the region of the aortic valve, at the region of the mitral valve, or at the region of any other valve. A delivery assembly comprising any delivery apparatus described throughout the current disclosure can be utilized, for example, to deliver a prosthetic aortic valve for mounting against the native aortic annulus or against a prosthetic valve previously implanted in a native aortic valve, to deliver a prosthetic mitral valve for mounting against the native mitral annulus or against a prosthetic valve previously implanted in a native mitral valve, or to deliver a prosthetic valve for mounting against any other native annulus or against a prosthetic valve previously implanted in any other native valve.

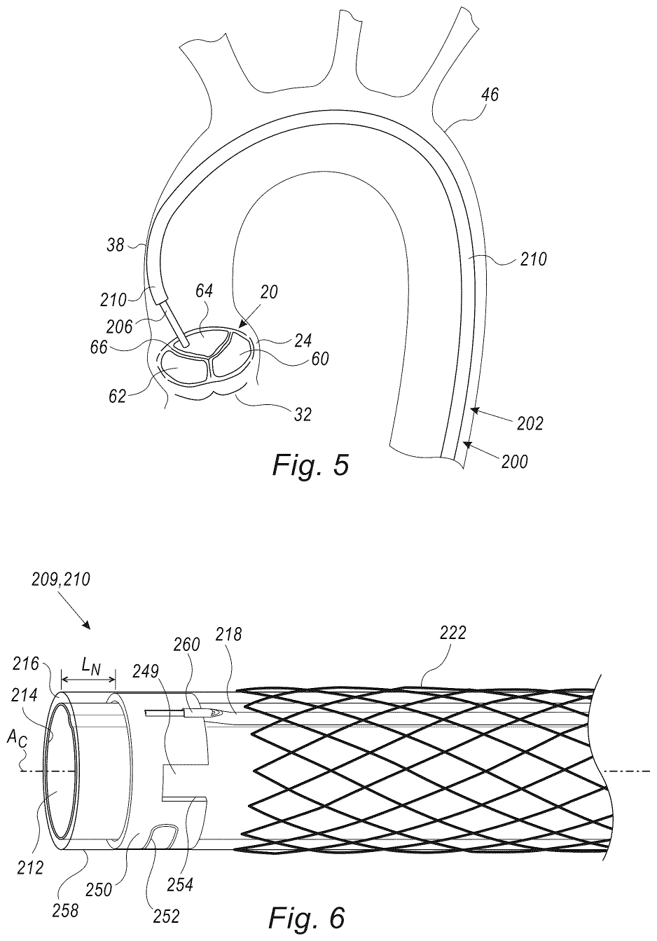

FIG. 4 illustrates an exemplary delivery assembly 200 that includes an exemplary delivery apparatus 202 adapted to deliver an inner catheter 208, wherein the inner catheter 208 can be part of a cutting or lacerating device for modifying a host leaflet, and/or part of an apparatus that can carry an implantable prosthetic device, such as prosthetic valve 100 described above with respect to FIGS. 2A-2B. In some examples, the delivery apparatus 202 includes a handle 204 and a steerable delivery catheter 210. An inner catheter 208 can extend through a primary lumen of the steerable delivery catheter.

The steerable delivery catheter 210 and the inner catheter 208 can be configured to be axially movable relative to each other. For example, or a distally oriented movement of the inner catheter 208 relative to the steerable delivery catheter 210, can expose a distal portion of the inner catheter 208.

The proximal ends of the steerable delivery catheter 210 and the inner catheter 208 can be coupled to the handle 204. During delivery of the prosthetic valve 100, the handle 204 can be maneuvered by an operator (for example, a clinician or a surgeon) to axially advance or retract components of the delivery apparatus 202, such as the inner catheter 208 or any other components passing therethrough, such as a cutting device which will be described in further detail below, and/or a balloon catheter equipped with an inflatable balloon (not shown) and carrying a prosthetic valve, through the patient's vasculature and/or along the target site of treatment.

The handle 204 can include a steering mechanism configured to adjust the curvature of the distal end portion of the delivery apparatus 202. In the illustrated example, the handle 204 can include an adjustment member, such as the illustrated rotatable knob 206a, which in turn is operatively coupled to the proximal end portion of a pull-wire. The pull-wire can extend distally from the handle 204 through the steerable delivery catheter 210 and has a distal end portion affixed to the steerable delivery catheter 210 at or near the distal end of the steerable delivery catheter 210. Rotating the knob 206a can increase or decrease the tension in the pull-wire, thereby adjusting the curvature of the distal end portion of the delivery apparatus 202. Further details on steering or flex mechanisms implemented in a handle 204 for controlling the tension of a pull-wire can be found in U.S. Pat. No. 9,339,384, which is incorporated by reference herein. The handle 204 can further include an adjustment mechanism including an adjustment member, such as the illustrated rotatable knob 206b. The adjustment mechanism can be configured to adjust the axial position of the inner catheter 208 relative to the steerable delivery catheter 210. The handle can include additional adjustment mechanisms controllable by additional knobs to maneuver additional components of the delivery apparatus 202, such as axial movement of other components and/or shafts of a cutting device and/or prosthetic valve deployment assemblies that can extend through the steerable delivery catheter 210.

In some examples, a delivery assembly 200 that includes a prosthetic valve can be packaged in a sterile package that can be supplied to end users for storage and eventual use. In some examples, the leaflets of the prosthetic valve (can be made from bovine pericardium tissue or other natural or synthetic tissues) are treated during the manufacturing process so that they are completely or substantially dehydrated and can be stored in a partially or fully crimped state without a hydrating fluid. In this manner, the package containing the delivery assembly can be free of any liquid. Methods for treating tissue leaflets for dry storage are disclosed in U.S. Pat. Nos. 8,007,992 and 8,357,387, both of which documents are incorporated herein by reference.

For illustrative purposes, the steerable delivery catheter 210 is described herein, in some examples, as having three longitudinal segments, i.e., a distal segment 210′, an intermediate segment 210″ which is proximal to the distal segment 210′, and a proximal segment 210′″ which is proximal to the intermediate segment 210′″. The segments 210′, 210″, 210′″ are integral or continuous segment of the steerable delivery catheter 210 but may have, in some examples, different constructions as described herein. It is to be understood that the steerable delivery catheter 210 can include additional segments proximal to the proximal segment 210′″ or distal to the distal segment 210′ without departing from the scope of the disclosure. Moreover, it is to be understood that each of the segments 210′, 210″, 210″ can be further divided into sub-segments that can include different materials or can include different constructions, and/or that the steerable delivery catheter can have any other number of segments, such as only a distal segment and a proximal extent continuous therewith, or a single segment extending the whole length of the steerable delivery catheter. The distal, intermediate, and proximal segments 210′, 210′, 210′ are the integral segments of the delivery catheter 210 that are sufficiently flexible to bend. However, the delivery catheter 210 may include additional segments, or any of the segments can have sub-segments or portions thereof, which are relatively rigid and not configured to flex or bend, as will be described in greater detail below.

The distal segment 210′ of a steerable delivery catheter 210 can be the segment that includes a slotted tube, as will be described in greater detail below for slotted tube 230 (or slotted tube 229 that will be described below for a distal segment 209′ of a steerable delivery catheter 209), such that the distal segment 210′ is the segment of the steerable delivery catheter 210 configured to be actively bent to steer the delivery catheter 210 and direct it towards a desired orientation, while the remainder to the steerable delivery catheter 210, extending proximally from the distal segment 210′, can be devoid of a slotted tube, configured to be rather passively bent during advancement through curved portions of the vasculature. Thus, the proximal end of the distal segment 210′ can be defined as the proximal terminal end of the slotted tube 230.

While three segments are described above, it is to be understood that in some examples, the steerable delivery catheter 210 can include two segments, namely the distal segment 210′ and the remainder of the steerable delivery catheter 210 extending proximally therefrom, meaning that the intermediate segment 210″ and the proximal segment 210′″ can be a single segment having uniform material properties along its length.

In some examples, the distal segment 210′ can have a length LC1, and the remainder of the steerable delivery catheter 210, extending proximally from the distal segment 210′, can have a length LC2. In some examples, such as when the portion of the steerable delivery catheter 210 extending proximally from the distal segment 210′ is further divided into intermediate segment 210″ and proximal segment 210′″, each optionally having different material properties, the intermediate segment 210″ can have a length LC21, and the proximal segment 210′″ can have a length LC22, such that the lengths LC21 and LC22 together define the length LC2. The combined length of segments, which is LC1+LC2, and in some examples, can be LC1+LC21+LC22, can be denoted LC_{T}. In some examples, the length LC1 of distal segment 210′ is in the range of 5-15% of the total length LC_{T}. In some examples, the length LC2 of the portion of the steerable delivery catheter 210 extending proximally from the distal segment 210′ is in the range of 85-95% of the total length LC_{T}. In some examples, such as when the intermediate segment 210″ and the proximal segment 210′″ can have non-uniform properties and geometries, the intermediate segment 210″ can be in the range of 5-10% of the total length LC_{T}, and the length LC3 of proximal segment 210′″ can be in the range of 75-90% of the total length LC_{T}. In some examples, the length L1 of the distal segment 210′ is between 90 and 100 mm. (millimeters). In some examples, the total length LC_{T} of the steerable delivery catheter 210 is in the range of 100 and 110 cm. (centimeters).

It is to be understood that while segments and lengths thereof are described above with respect to a steerable delivery catheter 210, equivalent segments and lengths thereof can be defined in the same manner for a steerable delivery catheter 209, mutatis mutandis.

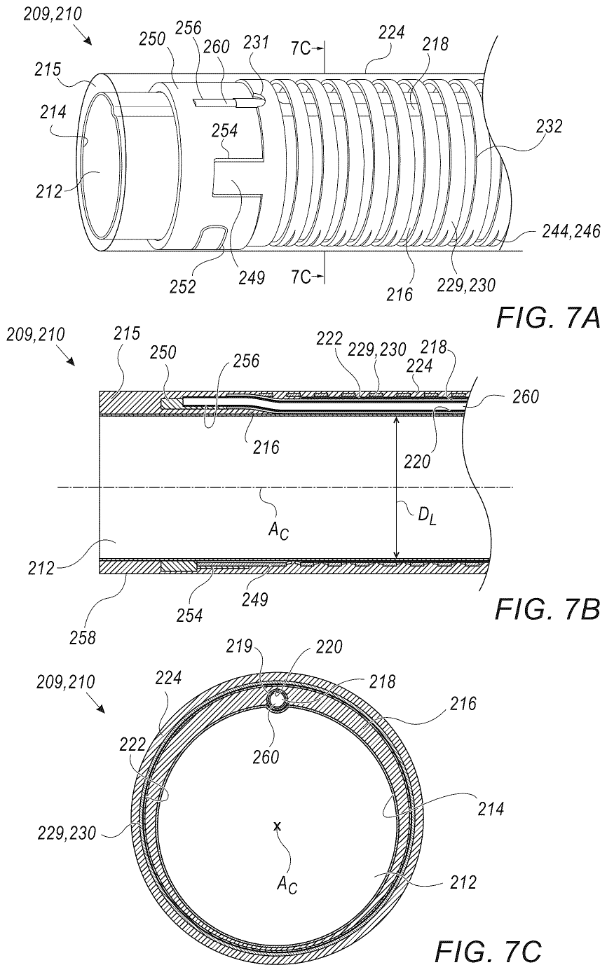

FIG. 5 shows a sectional view of a delivery assembly 200 being delivered in situ to a treatment site. When positioned in situ, steering of the delivery assembly 200 may be of importance in order to properly position a tool or prosthetic device at a desired location next to a site of treatment. The steerable delivery catheter is flexible and may be either passively bent, such as by being forced to articulate by the walls of the anatomical passageways, or actively steered, for example by manipulating a pull-wire as described above, to permit a user to navigate the delivery assembly 200 through a curved anatomy such as the aortic arch 46 as shown in FIG. 5.

Conventional steering mechanisms can assist the user in properly aligning a prosthetic valve carried by the delivery apparatus, within the target site, such as the native annulus. For example, the a prosthetic valve 100 needs to be properly aligned, axially and annularly/circumferentially, so that the prosthetic valve 100 properly engages the native annulus of the target site, e.g., the aortic annulus 24, or a previously implanted prosthetic valve in case of ViV procedures, without causing conduction blockages by implanting too deep (e.g., the prosthetic valve positioned too far towards the left ventricle) or causing an embolization of the prosthetic valve 100 because it was implanted too high (e.g., the prosthetic valve positioned not far enough towards the left ventricle). Some types of steering mechanisms allow the distal end portion of the delivery apparatus to bend in a predefined direction along a two-dimensional plane defined by the central longitudinal axis of the steerable catheter. If it is desired to bend or deflect the distal portion of the delivery apparatus in a different or even an opposing direction, the steerable delivery catheter may be torqued or rotated and then the pull-wire may be actuated to bend the distal portion of the steerable delivery catheter. Torquing the delivery apparatus 202 can be accomplished via rotation of the handle 204, thus permitting the user to circumferentially align the prosthetic valve 100 within the target site, e.g., the native annulus, in situ.

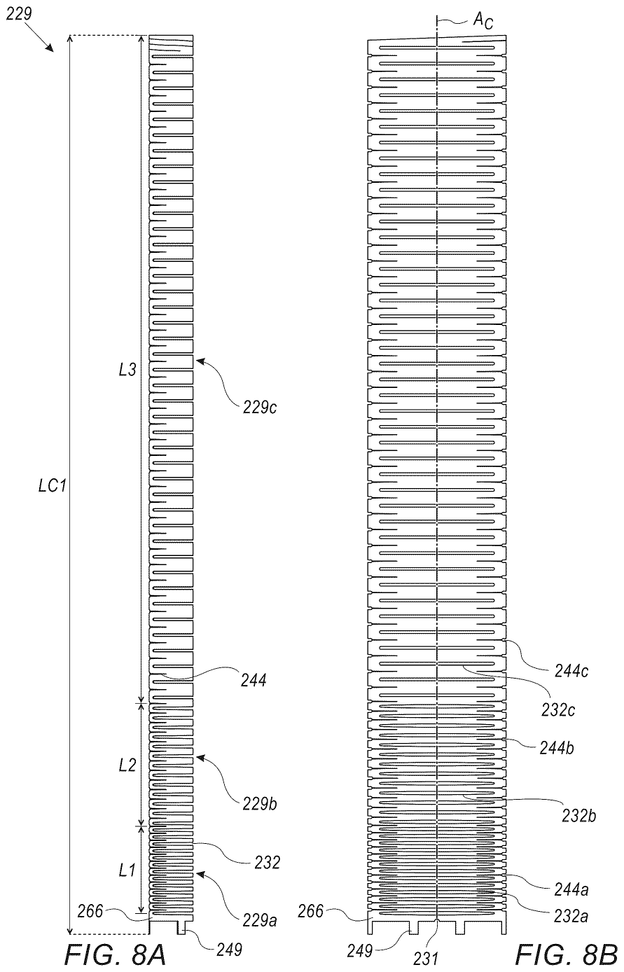

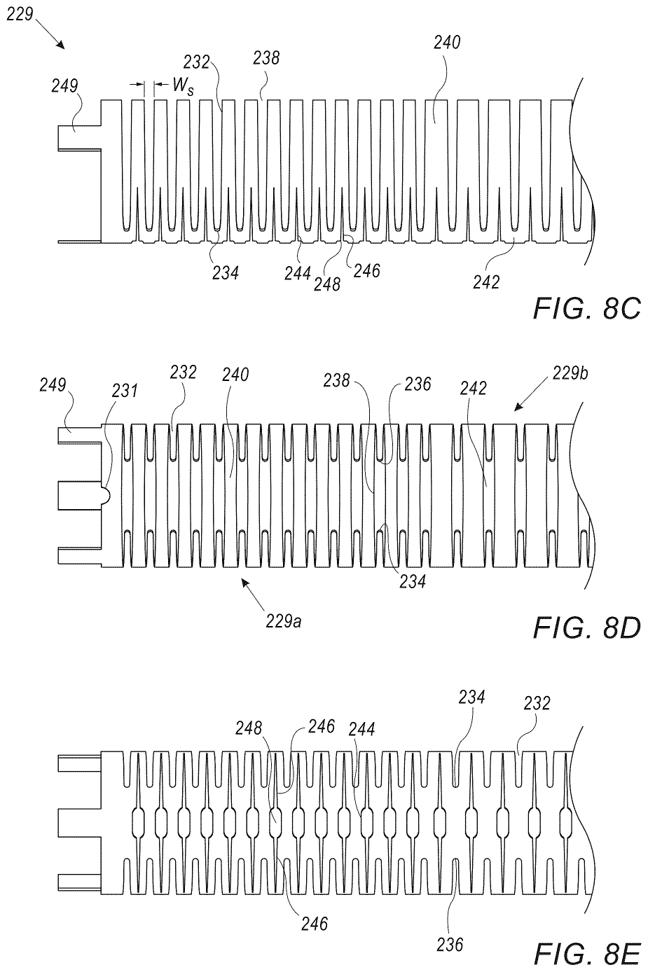

FIG. 6 shows a distal portion an exemplary steerable delivery catheter, with some components and/or layers thereof removed from view. The exposed portion illustrated in FIG. 6 can include components and/or layers of a steerable delivery catheter which can be either a steerable delivery catheter 209 configured to bend in a two-dimensional plane defined by a central longitudinal axis A_{C} thereof, or a steerable delivery catheter 210 configured to bend in a three-dimensional out-of-plane manner. Layers and/or components shown in FIG. 6 can be shared by both types of steerable delivery catheters 209 and 210. FIGS. 7A and 7B show a perspective view and a longitudinal sectional view, respectively, of a distal segment of an exemplary steerable delivery catheter 209 configured for planar articulation. FIG. 7C is a lateral cross-sectional view along line 7C-7C of FIG. 7A. The components and structure of steerable delivery catheter 209 shown in FIG. 7A-7C and described below, can be similarly implemented for any exemplary steerable delivery catheter 210 described herein, except for the arrangement of slots along a slotted tube, described in greater detail below, and optionally except for the path or orientation of a pull-wire lumen and a pull-wire extending therethrough, also described in greater detail below.

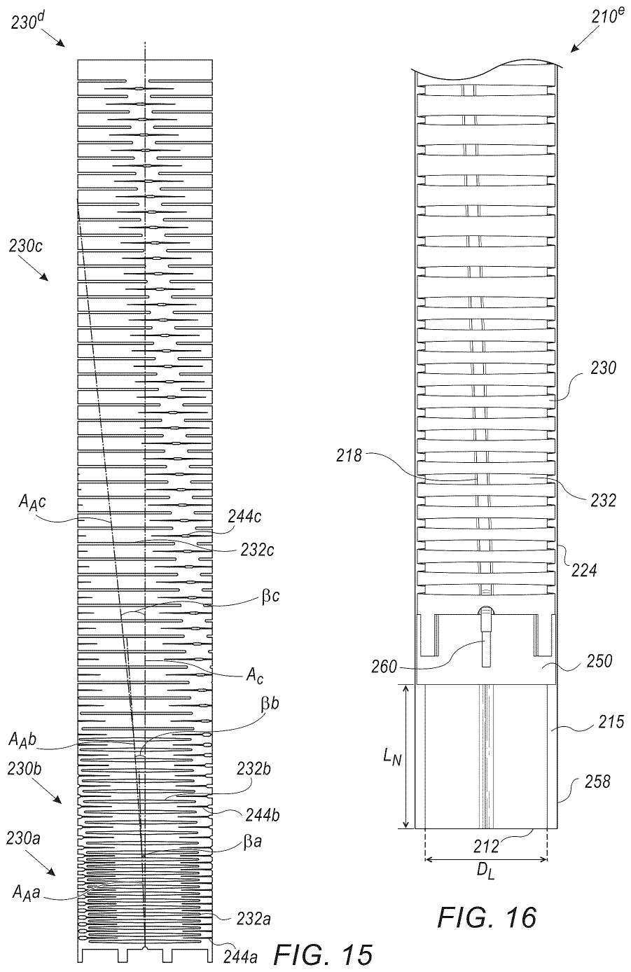

A steerable delivery catheter 209, 210 has a primary lumen 212 that extends the length of the catheter 209, 210, defining a central longitudinal axis A_{C}, for example, as shown in FIG. 7B. The primary lumen 212 can be used to transport one or more of a medical device (such as a prosthetic valve 100), tools (such as a cutting or perforating device 270 that will be described in greater detail with respect to FIGS. 17A-17D below), medicament, or other substance. The steerable delivery catheter 209 or 210 further includes a pull-wire lumen 218 through which a pull-wire 260 extends. The pull-wire lumen 218 is defined or preformed in a sidewall of the steerable delivery catheter 209, 210, such as in a polymeric layer 215 of catheter 209, 210. As shown, the pull-wire lumen 218 is radially offset from the central longitudinal axis A_{C}, and has a diameter that is significantly less than diameter D_{L} of the primary lumen 212. In some examples, the pull-wire 260 can include aramid fiber, carbon, or another relatively hard polymeric material. In some examples, the pull-wire 260 can include Nitinol or stainless steel.

Steerable delivery catheter 209, 210 can include a plurality of layers comprising a variety of different materials configured to impart various properties to the catheter 209, 210. In some examples, a low-friction and/or flexible primary lumen liner 214 can cover the inner surface around primary lumen 212, which can be made of (or coated by) a lubricious material such as polytetrafluoroethylene (PTFE), and can extend the full length of the primary lumen 212 of the delivery catheter 209, 210. The primary lumen liner 214 fully surrounds the interior surface of the primary lumen 212, to provide a smooth surface so that an inner catheter 208 and/or any other device, such as a perforating device 270 or a prosthetic valve 100, can be easily passed therethrough.

Steerable delivery catheter 209, 210 can include a polymeric layer 215 radially outward to the primary lumen liner 214. In some examples, a polymeric layer 215 can be comprised of at least two layers that can be fused to each other: an encapsulating polymeric layer 216 that can be disposed around the primary lumen liner 214, and an outer polymeric layer 224 which is radially outward to the encapsulating polymeric layer 216. The polymeric layer 215, and optionally an encapsulating polymeric layer 216 thereof, can encapsulate the pull-wire lumen 218 along at least some, and optionally along most, of the length of the steerable delivery catheter 209 or 210. The pull-wire lumen 218 can be defined or preformed in a sidewall of the steerable delivery catheter 209, 210, such as in a polymeric layer 215 of catheter 209, 210. The polymeric layer 215, or a sub-layer thereof, such as the encapsulating polymeric layer 216, can have a non-uniform cross-sectional thickness to provide a thicker wall section for the pull-wire lumen 218, which is sized and dimensioned to receive the pull-wire 260.

The pull-wire lumen 218 can be formed within the polymeric layer 215, such as within encapsulating polymeric layer 216, as an elongated radial protrusion that axially extends along the primary lumen liner 214 and radially extends or protrudes into the primary lumen 212, as illustrated in FIG. 7C. The pull-wire lumen 218 can be offset or off-centered within the polymeric layer 215 such that the pull-wire lumen 218 is disposed closer to the primary lumen 212 than an outer surface of the steerable delivery catheter 209, 210.

The pull-wire lumen 218 is sized and dimensioned to receive the pull-wire 260. As mentioned above, the pull-wire 260 is operable to bend the steerable delivery catheter 209, 210, for example for steering an inner catheter 208 extendable therethrough, and may be selectively tensioned by the operator. The pull-wire 260 is disposed within the pull-wire lumen 218 such that it can be selectively tensioned by the operator to bend the distal portion of delivery catheter 209, 210. The term “slidably”, as used herein, refers to back and forth movement in a longitudinal direction, which can be generally parallel to, and/or somewhat angled relative to, the central longitudinal axis A_{C}. In some examples, the pull-wire lumen 218 is defined by a sleeve 219 (indicated, for example, in FIG. 7C) which is sufficiently flexible yet substantially axially non compressible. For example, a sleeve 219 can comprises an elongated slotted tube (such as a metallic hypotube) that has a plurality of axially-spaced, circumferentially extending slots formed (such as by laser cutting) along a length of the tube. The term “tensioned”, as used herein, refers to a pull-wire 260 being proximally pulled such that an initial positive tension force is imposed thereon, as contrasted to an untensioned pull-wire 260 having no axial force applied thereto.

A conduit defining the pull-wire lumen 218 can include low-friction and/or flexible pull-wire lumen liner 220 covering the inner surface of the pull-wire lumen 218, and can comprise ePTFE, PTFE or another suitable material to reduce friction between the conduit and the pull-wire 260.

As mentioned above, the steerable delivery catheter 209, 210 can include, in some examples, an outer polymeric layer 224 disposed radially outwards of the encapsulating polymeric layer 216 and the pull-wire lumen 218, such that the encapsulating polymeric layer 216 and the outer polymeric layer 224 can together form a combined polymeric layer 215. In some examples, the encapsulating polymeric layer 216 and the outer polymeric layer 224 are made of the same material. In some examples, the material properties of the outer polymeric layer 224 can be different from those of the encapsulating polymeric layer 216. In some examples, the outer polymeric layer 224 can have a uniform thickness around the circumference of the central longitudinal axis A_{C}. Any of the polymeric layer 215, encapsulating polymeric layer 216, and/or outer polymeric layer 224 can comprise, for example, any of a variety of polymeric materials such as polyamides (e.g., VESTAMID®), polyether block amides (e.g., Pebax®), nylon, or any other suitable biocompatible polymer or combinations thereof along its length.

In some examples, steerable delivery catheter 209, 210 can include a braid 222, shown for example in FIG. 6, but removed from view in FIG. 7A for clarity. In some examples, the braid 222 can be embedded in the polymeric layer 215. In some examples, the braid 222 can be disposed radially outward the encapsulating polymeric layer 216 and the pull-wire lumen 218. In some examples, the braid 222 can be disposed between the encapsulating polymeric layer 216 and the outer polymeric layer 224. The braid 222 can be composed of, in some examples, metal wires (for example, stainless steel or titanium flat wires) braided together in a pattern, such as a one-over, one-under woven pattern as illustrated in FIG. 6, or any other pattern.

The braid 222 may be formed of a material such as stainless steel, Nitinol, tungsten, carbon, aramid, glass fiber, a stainless steel that has been reinforced with polyimide, or molybdenum. In some examples, the material utilized for the braid 222 has an ultimate tensile strength (UTS) in the range of 200,000 to 350,000 lbs/in². In some examples, the material utilized for the braid 222 is high temper stainless steel type 2913B ribbon wire.

The braid 222 can be configured to resist spontaneous torsional deformation of the steerable delivery catheter 209, 210 during delivery to allow the catheter to transmit torque, which can aid in positioning an inner catheter 208 extending therethrough at the treatment site. The braid 222 can also provide crush or kink-resistance properties to steerable delivery catheter 209, 210.

Steerable catheter 209 can have distal, intermediate and proximal segments 209′, 209″ and 209′″, respectively (see FIG. 4), which can be generally characterized in a manner similar to that described above for segments 210′, 210″ and 210″ for of steerable catheter 210. In some examples, the stiffness of the steerable delivery catheter 209, 210 varies along its length. In some examples, at least one segment of a steerable delivery catheter 209, 210 can have a stiffness that is different from that of at least one other segment thereof. In some examples, the polymeric layer 215 of the distal segment 209′, 210′ of a steerable delivery catheter 209, 210 comprises a first polymer having a first stiffness. In some examples, the polymeric layer 215 of the intermediate segment 209″, 210″ of a steerable delivery catheter 209, 210 comprises a second polymer having a second stiffness. In some examples, the polymeric layer 215 of the proximal segment 209′″, 210′″ of a steerable delivery catheter 209, 210 comprises a third polymer having a third stiffness. In some examples, the first stiffness of the polymeric layer 215 along the distal segment 209′, 210′ is less than the second stiffness of the polymeric layer 215 along the intermediate segment 209″, 210″, and the second stiffness is less than the third stiffness of the polymeric layer 215 along the proximal segment 209″, 210″″. In some examples, the first polymer along the distal segment 209′, 210′ comprises PEBAX® 35D. In some examples, the second polymer along the intermediate segment 209″, 210″ comprises PEBAX® 63D. In some examples, the third polymer along the proximal segment 209′″, 210′″ comprises VESTAMID® ML21.

In some examples, the distal segment 210′ can be further divided to sub-segments, each comprising a material having a different diameter. For example, the distal segment 210′ can include a first sub-segment that comprises PEBAX® 25D, a second sub-segment proximal to the first sub-segment, comprising PEBAX® 35D, and a third sub-segment, proximal to the second subsegment, comprising PEBAX® 55D (first, second and third sub-segment not annotated).

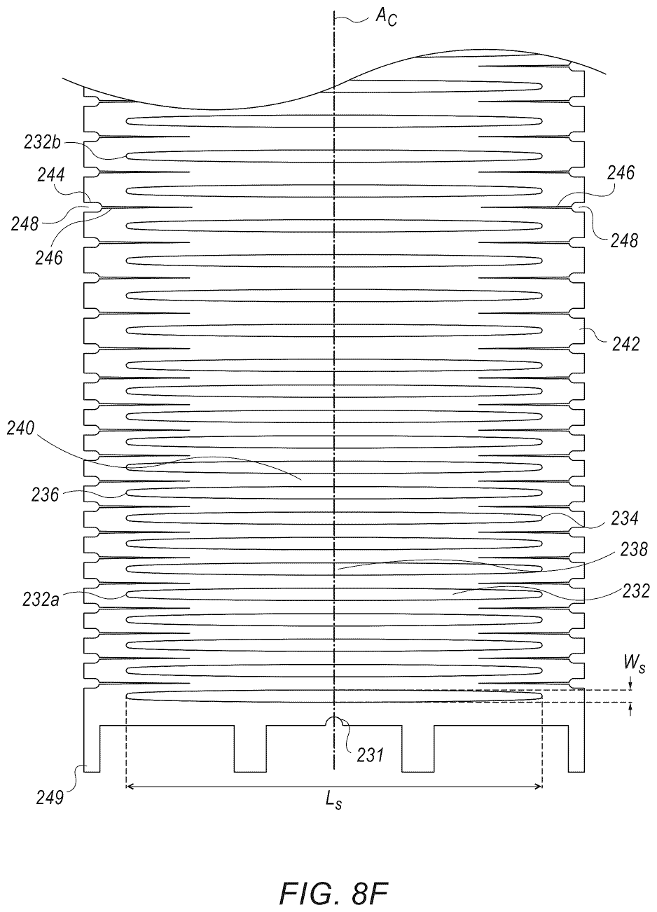

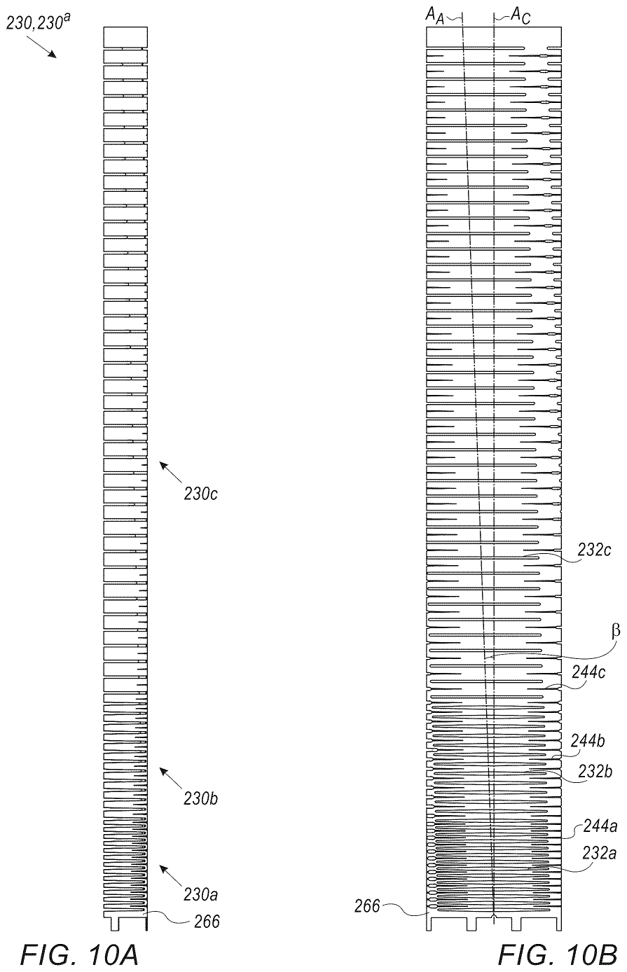

A steerable delivery catheter may further comprise a slotted tube which is configured for elastic deformation. Steerable delivery catheter 209 includes a slotted tube 229 which will be described in greater detail below, with respect to FIGS. 8A-8F for example. Steerable delivery catheter 210 includes a slotted tube 230 which will be described in greater detail below, with respect to FIGS. 10A-10F for example. In some examples, the slotted tube 229, 230 is embedded in the polymeric layer 215, along at least part of the length LC1 of the distal segment 209′, 210′. In some examples, the slotted tube 229, 230 can be disposed radially outward the encapsulating polymeric layer 216 and the pull-wire lumen 218. In some examples, the slotted tube 229, 230 can be disposed between the encapsulating polymeric layer 216 and the outer polymeric layer 224. In the illustrated example, the slotted tube 229, 230 overlays the braid 222. However, it is to be understood that in some example, the braid 222 can overlay the slotted tube 229, 230. In some examples, a steerable delivery catheter can be devoid of a braid. For example, the polymeric layer can be formed of a material which is compliant and flexible while still maintaining a sufficient degree of column strength to resist buckling of the delivery catheter, and sufficient tear resistance to reduce the likelihood that a component extending through the steerable delivery catheter will cause the catheter to tear.

The pull-wire 260 can exit the pull-wire lumen 218 at a distal end thereof, and be coupled at its distal end to a pull ring 250. In some examples, the pull ring 250 can be embedded in the polymeric layer 215, optionally between the encapsulating polymeric layer 216 and the outer polymeric layer 224. Pull ring 250 can be disposed inside the distal segment 210′, 210″, distal to the slotted tube 229, 230, and can include one or more cut-outs 254 aligned with corresponding one or more tube teeth 249 of the slotted tube 229, 230, situated therein. The pull-wire 260 is fixedly attached to the pull ring 250, which is embedded inside the distal part of the steerable delivery catheter. Pull-wire 260 can be, for example, welded to the pull ring 250, optionally at a groove 256 of the pull ring 250.

During manufacturing of a steerable delivery catheter, the primary lumen liner 214 can be formed, for example around a mandrel (not shown), over which an encapsulating polymeric layer 216 can be formed. The pull-wire lumen 218 can be placed over or formed inside of the encapsulating polymeric layer 216, and a braid 222 can be formed in a desired pattern around the encapsulating polymeric layer 216 and the pull-wire lumen 218. The pull ring 250 can be placed around the encapsulating polymeric layer 216, distal to the braid 222, as illustrated in FIG. 6. The pull ring 250 can include a wire groove 256, through which the pull-wire 260 and/or pull-wire lumen 218 can extend radially outward and over a portion of the pull ring 250, and the distal end of the pull-wire 260, extending out of the pull-wire lumen 218, can be affixed to the pull ring, such as by welding, gluing, or any other suitable manner.

The slotted tube can be placed around the encapsulating polymeric layer 216 and the pull-wire lumen 218, optionally over the braid 222, and one or more tube teeth 249 extending from the tube distal end 266 can be positioned in alignment with corresponding cut-outs 254 of the pull ring 250. The outer polymeric layer 224 can then be formed over the other layers and components, encapsulating the slotted tube 229, 230, the braid 222, and/or the pull-wire lumen 218, inside the resulting polymeric layer 215, between the encapsulating polymeric layer 216 and the outer polymeric layer 224. The pull ring 250 can further include, in some examples, one or more windows 252 about its circumference, such that when the polymeric material of the outer polymeric layer 224 is reflowed over the pull ring 250, the molten material can flow through the windows 252 to encapsulate the pull ring 250 in the polymeric layer 215. The slotted tube can be similarly reflowed with the polymeric material to facilitate flexibility, as the polymeric material fills the voids formed by the laser cut pattern of the slotted tube, including slots 232, and when present, opposite cuts 244, such that both polymeric layers 224 and 216 can be sintered together during manufacturing, fully covering the slotted tube. The polymeric material may be similarly reflowed during manufacturing, through the spaces of the braid 222.

Pull ring 250 can be provided either as a stand-alone component attachable to the polymeric layer 215 and/or slotted tube 229, 230, or can be integrally formed with the slotted tube 229, 230, in which case it can be defined as part of the tube distal end 266. While pull ring 250 is described and illustrated in some examples herein as a separate component coupled, directly or indirectly, to the slotted tube, it is to be understood that this is not meant to be limiting, and that any reference to a pull-ring 250 throughout the specification and the claims, similarly refers to a distal end portion 266 of the slotted tube itself, devoid of cut-outs 254 and/or tube teeth 249, to which the pull-wire 260 can be coupled, such as by welding, gluing, and the like.

Pull-wire 260 is coupled to the slotted tube 229, 230 either directly or indirectly. Any reference to a pull-wire 260 coupled to the slotted tube 229, 230 throughout the specification and the claims, refers to a distal end of the pull-wire 260 being attached either directly to a distal end of the slotted tube, or to another component (such as a separate pull-ring 250 or part of the polymeric layer 215) which is attached, in turn, to a distal end of the slotted tube. While the pull-wire 260 is described and illustrated in some examples herein to be indirectly attached to a distal end of the slotted tube by being affixed to a pull-ring 250 which, in turn, is coupled to the distal end of the slotted tube, it is to be understood that the pull-wire 260 can be, in some examples, directly attached to the slotted tube 229, 230, such as when the pull-ring 250 is provided as an integral distal end portion of the slotted tube itself. Similarly, the pull-wire 260 can be indirectly attached to a distal end of the slotted tube 229, 230 by having its distal end coupled to (for example, embedded in) a distal region of the polymeric layer 215, which is in turn coupled to the slotted tube (for example, by having the slotted tube embedded inside the polymeric layer 215)

As mentioned above, a steerable delivery catheter can include segments or sub-segments that are not necessarily configured to bend. In some examples, the proximal segment 209′″, 210′″ may include a proximal non-steerable sub-segment that is configured to permit passage of a pull-wire into the handle 204, and such a proximal sub-segment can be configured to be relatively rigid and not sufficiently flexible to bend. While the pull-wire 260 is primarily housed or disposed with the pull-wire lumen 218, the proximal segment 209′″, 210′″ can include an exit point (not shown) from which the proximal end of the pull-wire can radially extend out of the polymeric layer 215, and continue further therefrom into the handle 204 to be pulled, which results in controlled bending movement of the steerable distal segment 209′, 210′ of the delivery catheter.

In some examples, the distal segment 209′, 210′ may include a tip 258 distal to the slotted tube 229, 230 that is configured to be relatively rigid and not sufficiently flexible to bend, to provide structural support to a component extendable through and out of the steerable delivery catheter, such as an inner catheter 208. The tip 258 can include a polymeric layer 215 distal to the pull ring 250 along a length LN, that is devoid of the slotted tube 229, 230, the braid 222, and/or the pull-wire lumen 218. In some examples, the tip comprises polymeric material of higher durometer than another sub-segment of the distal segment 209′, 210′ proximal thereto. In some examples, the tip 258 comprises PEBAX® 55D. In some examples, the length L_{N} of the tip 258 is less than the lumen diameter D_{L}.

FIG. 8A shows a side view of an exemplary slotted tube 229, configured for planar articulation. FIG. 8B shows the slotted tube 229 of FIG. 8A in a flattened configuration, illustrating the manner by which the slotted tube is cut if the tube were to be longitudinally cut along its length to form slots 232 and laid flat. For illustrative purposes, the slotted tube 229 is described herein, in some examples, as having three longitudinal portions, i.e., a distal portion 229a terminating at a distal end 266 of the tube, an intermediate portion 229b which is proximal to the distal portion 229a, and a proximal portion 229c which is proximal to the intermediate portion 229b. The portions 229a, 229b, 229c are integral or continuous portions of the slotted tube 229 but may have, in some examples, different constructions as described herein. In some examples, the distal segment 209′ of steerable catheter 209 includes all three portions 229a, 229b and 229c of the slotted tube 229. In some examples, the distal segment 209′ of steerable catheter 209 includes the distal portion 229 of the slotted tube 229, while the intermediate 229b and proximal 229c portions can extend further along the intermediate segment 209″, an optionally further along at least part of the proximal segment 209′″ of catheter 209.

In some examples, the slotted tube 229 is comprised in the distal segment 209′ of the steerable delivery catheter 209. In some examples, the slotted portions 229a, 229b, and 229c are comprised in the distal segment 209′ of catheter 209. The distal portion 229a of the slotted tube 229, can extend along a length L1; the intermediate portion 229b of the slotted tube 229 can extend along a length L2; and the proximal portion 229c of the slotted tube 229 can extend along a length L3. In some examples, the length LC1 of the distal segment 209′ of catheter 209′ is greater than the sum of the lengths L1+L2+L3 of the slotted portions 229a, 229b, and 229c of slotted tube 229.

FIGS. 8C, 8D and 8E show a side view, a top view and a bottom view, respectively, of a distal region of the slotted tube 229 of FIG. 8A, including the distal portion 229a and part of the intermediate portion 229b continuously extending therefrom. FIG. 8F shows the distal region of the slotted tube 229 of FIG. 8C in a flattened configuration, illustrating the manner by which the distal region of the slotted tube is cut if the tube were to be longitudinally cut along its length to form slots 232 and laid flat. The slotted tube 229 can be formed by seamless drawn tubing where slots are laser cut into the tube.

A slotted tube 229, 230 can be made of a metal, such as stainless steel, Nitinol, or any other suitable material. As mentioned above, a slotted tube 229, 230 can optionally include one or more tube teeth 249 extending distally from a distal edge thereof, configured to extend into corresponding cut-outs 254 formed in the pull ring 250. In some examples, such as when the slotted tube 229, 230 is formed of stainless steel, the tube teeth 249 can be affixed to the pull ring 250, such as be welding. In some examples, the tube teeth 249 are situated inside the cut-outs 254 without being directly affixed thereto, held in place by the polymeric layer 215 disposed therearound. In some examples, a slotted tube 229, 230 further comprises a semi-circular cut-out 231 that can be aligned with a wire groove 256 of the pull ring 250, to allow pull-wire 260 and/or pull-wire lumen 218 to extend therethrough, such as from a position beneath (i.e., radially inward to) the slotted tube 229, 230, to a position above (i.e., radially outward to) the pull ring 250.

A slotted tube 229, 230 comprises a plurality of successive discrete slots 232 which are cut (such as by laser-cutting or any other suitable manufacturing procedure) through the wall thickness of the slotted tube. Each of the slots 232 extends in a transverse direction of the tube 229, 230 from a slot first circumferential end 234 to a slot second circumferential end 236, spanning more than 180° of the circumference of the tube 229, 230 around the central longitudinal axis A_{C}. In some examples, slot 232 spans more than 270° around the central longitudinal axis A_{C}. In some examples, slots 232 extend around the circumference of the slotted tube, for example over at least 200°, at least 220°, at least 280°, at least 300°, at least 320°, or at least 340° circumferentially, leaving an uncut gap between the slot circumferential ends 234, 236 that defines the backbone 242.

As shown in, for example, in FIG. 8F, each slot 232 has a slot length L_{S} defined between the slot first circumferential end 234 and the slot second circumferential end 236, and a slot width W_{S} in a direction perpendicular to the slot length L_{S}. The slot length L_{S} is much greater than the slot width W_{S}. In some examples, the slot length L_{S} extends along the circumference of the slotted tube, and the slot width W_{S} is parallel to the central longitudinal axis A_{C}. A slot circumferential center 238 can be defined between the slot first circumferential end 234 and the slot second circumferential end 236. Slots 232 define ribs 240 extending axially therebetween. The portion of the tube 229, 230 not cut by the slots 232 may be referred to as a backbone 242 of the slotted tube.

In some examples, a slotted tube 229, 230 can further comprise a plurality of opposite cuts 244 that extend through the backbone 242 of the tube 229, and may partially extend along parts of the ribs 240, but do not extend through the portions of the ribs 240 that are directly opposite to the backbone 242. Each opposite cut 244 includes an opening 248 aligned with the center of the backbone 242, and two slits 246 that extend circumferentially from both ends of the opening 248, passing over parts of the ribs 240 that do not reach the portions which are opposite to the opening 248. Stated another way, the uncut portions of the ribs 240 are generally opposite to the openings 248. As shown, the opening 248 of an opposite cut 244 can be wider (in the longitudinal direction) than the slits 246 extending therefrom.

The distal portion 229a of slotted tube 229 can include a plurality of distal slots 232a and a plurality of distal opposite cuts 244a, the intermediate portion 229b of slotted tube 229 can include a plurality of intermediate slots 232b and a plurality of intermediate opposite cuts 244b, and the proximal portion 229c of slotted tube 229 can include a plurality of proximal slots 232c and a plurality of proximal opposite cuts 244c. In some examples, the plurality of distal slots 232a can be equally spaced from each other along the longitudinal direction (i.e., along the direction of central longitudinal axis A_{C}). In some examples, the plurality of intermediate slots 232b can be equally spaced from each other along the longitudinal direction. In some examples, the plurality of proximal slots 232c can be equally spaced from each other along the longitudinal direction. The opposite cuts 244 can be generally positioned midway between adjacent slots 232 along the longitudinal direction, though other positions between neighboring slots 232 are contemplated.

In some examples, the slots 232 along the length of the slotted tube 229 are equally spaced from each other, such that the ribs 240 formed therebetween have similar widths (defined as the dimension of rib 240 between two adjacent slots 232 in a longitudinal direction) along the entire length of the slotted tube 229. In some examples, the slots 232 of at least one portion of the slotted tube 229 can be differently spaced from each other relative to the slots 232 of at least one other portion of the tube 229, such that the ribs 240 along different portions can be wider or narrower to change the radius of the greatest possible bend by the corresponding bending portions. In the illustrated example, the ribs 240 of the distal portion 229a are narrower than the ribs 240 of the intermediate portion 229b, and the ribs 240 of the intermediate portion 229b are narrower than the ribs 240 of the proximal portion 229c. This will result in variable flexibility of the slotted tube 229 such that the distal portion 229a is the most flexible.

In some examples, the plurality of distal slots 232a can be similarly shaped and dimensioned with respect to each other. In some examples, the plurality of intermediate slots 232b can be similarly shaped and dimensioned with respect to each other. In some examples, the plurality of proximal slots 232c can be similarly shaped and dimensioned with respect to each other. In some examples, the shape and dimensions of the slots 232 along the length of the slotted tube 229 are similar to each other. In some examples, the slots 232 of at least one portion of the slotted tube 229 can be differently shaped and/or dimensioned with respect to slots 232 of at least one other portion of the tube 229, such that the different shapes and/or dimensions of the slots can change the radius of the greatest possible bend by the corresponding bending portions.

In some examples, the slot width W_{S} can increase in size from the slot's first and second circumferential ends 234, 236 towards the slot circumferential center 238, thus forming an extended ovaloid shape. In some examples, the slot width W_{S} can be uniform between the slot's first and second circumferential ends 234, 236. In the illustrated examples, the distal slots 232a and intermediate slots 232b are shown to be ovaloid-shaped slots, having a slot width W_{S} at their circumferential centers 238 greater than at their first and second ends 234, 236, while the proximal slots 232c are shown to have a uniform slot width W_{S}.

As mentioned above, the curvature of one or more portions of the steerable delivery catheter can be changed based on the operator manipulating the pull-wire 260 via an actuator of the handle. In the example illustrated in FIG. 4, the actuator is a knob 206a that is rotatable relative to the housing of the handle 204. When the knob 206a is rotated in a first direction (i.e., one of clockwise or counterclockwise), the pull-wire 260 is retracted and placed under tension to bend or deflect the distal portion of the steerable delivery catheter. Stated another way, when the pull-wire 260 is retracted via actuation of the actuator 206a, the pull-wire 260 is placed under tension and bends the distal segment 209′, 210′. When the knob 206a is rotated in a second direction opposite from the first direction (i.e., the other of clockwise or counterclockwise), tension on the pull-wire 260 is released and the distal portion of the steerable delivery catheter resumes its straightened (or passively bent) configuration.

Bending of the steerable delivery catheter, such as by pulling on the pull-wire 260, results in the ribs 240 being moved closer together at the portions opposite to the backbone 242. The slots 232 can be narrowed or closed at the slot circumferential centers 238, bending the delivery catheter in the direction of the closure. When present, the opposite cuts 244, including openings 248 thereof, can open as the delivery catheter is bent, such as along the backbone 242. The backbone 242 flexes during bending of the delivery catheter. The backbone 242 can be configured to resist axial compression of the slotted tube, for example when pushing the steerable delivery catheter along the vasculature of a patient or when bending the steerable delivery catheter along a direction that is not in line with the predetermined bending direction of the respective bending portion. The slits 246 are configured to operate similar to relief cuts in that the slits 246 are closed or substantially closed when the slotted tube 229, 230 is in a straight configuration, and can open or expand when the ribs 240 move toward one another as the slotted tube 229, 230 is transitioned into a bent configuration.

Resilience of the slotted tube material can be configured to assist the steerable delivery catheter in returning to a straight or unbent condition (or less bent) condition, when the pull-wire 260 is released (i.e., no longer tensioned, or tensioned at a smaller pulling force). The shape of the slots and/or cuts and the material from which the slotted tube is made, such as Nitinol in some examples, can facilitate “spring-back” of the slotted tube to its pre-bent configuration. This can be advantageous because, in some examples, the pull-wire 260 will not be compressed, thus avoiding kinks.

The slots 232 of the slotted tube 229 of steerable delivery catheter 209 are circumferentially aligned with each other along the entire length of the slotted tube 229. This means that the slot circumferential centers 238 are positioned at the same angular orientation or circumferential position relative to each other, with respect to the central longitudinal axis A_{C}, such that an imaginary axis extending through the slot circumferential centers 238 is parallel to the central longitudinal axis A_{C}. The opposite cuts 244 of the slotted tube 229 can be similarly aligned with each other, such that the backbone 242 of slotted tube 229 is parallel to the central longitudinal axis A_{C}. If all slots 232 of slotted tube 229 have identical slot lengths L_{S}, the slot first circumferential ends 234 can be similarly aligned with each other, and the slot second circumferential ends 236 can be similarly aligned with each other. The pull-wire lumen 218 of steerable delivery catheter 209, and the pull-wire 260 extending therethrough, is aligned with the slot circumferential centers 238, such that the pull-wire lumen 218 is parallel to the central longitudinal axis A_{C}. This aligned configuration of the slots 232 and the pull-wire lumen 218, parallel to the central longitudinal axis A_{C}, will generally result in a single plane of motion during bending of the slotted tube 229, which will be generally aligned with the slot circumferential centers 238. Accordingly, when force is applied to the delivery catheter 209 by tensioning the pull-wire 260, the aligned pattern of the slots 232 of tube 229 will bend the catheter 209 along the plane formed by the slot circumferential centers 238, allowing for a two-dimensional movement.

It is to be understood that the steerable delivery catheter in not only actively bendable due to pulling of a pull-wire 260, but can be also passively bendable, configured to bend in a similar manner, when the catheter is forced to conform to the shape of the vascular path when it impacts the anatomical walls during advancement thereof.

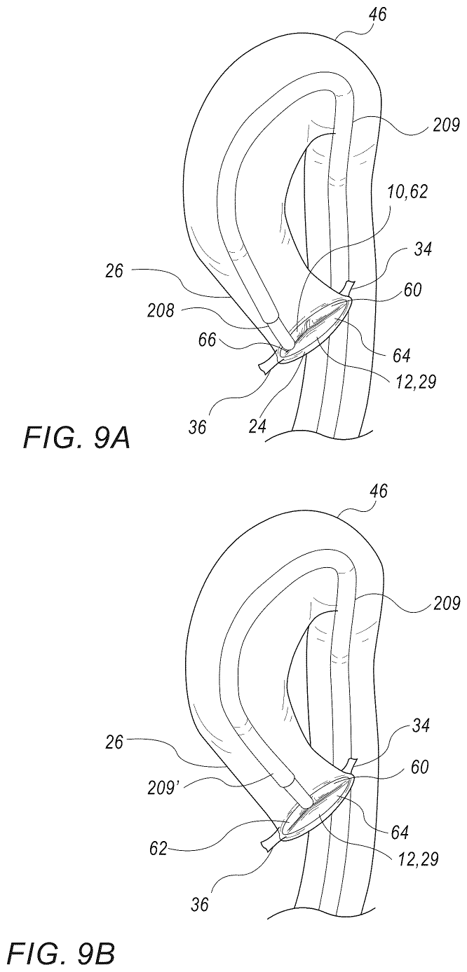

FIG. 9A shows a steerable delivery catheter 209 advanced, along the aortic arch 46, towards the native aortic valve 22. In some cases, such as when the catheter 209 is allowed to passively bend due to interaction with the native anatomy without being actively bent by pulling the pull-wire 260, as the steerable delivery catheter 209 is pressed against the aortic wall 38 during advancement through the curved aortic arch 46, the anatomical structure of the aortic arch 46 orients the distal end of the steerable delivery catheter 209 towards a portion of the aortic root 22 adjacent to the right coronary ostium 44, such as the right/non commissure 66 (disposed between the right leaflet 62 and the non-coronary leaflet 64) or other regions of the right leaflet 62 or non-coronary leaflet 64. This position may be different from a desired site of treatment in a leaflet modification procedure (e.g., modifying a left leaflet 60 to prevent obstruction of the left coronary ostium 42). Thus, it may be desired, in some occasions, to be able to direct or steer the distal end of the steerable delivery catheter, and any component extendable therethrough, such as an inner catheter 208, to a region that is different from the region at which the distal end of the steerable delivery catheter lands due to the native anatomy of the patient.

When positioned in situ, steering and torquing of the delivery assembly 200 are of utmost importance in order to properly position the distal portion of the steerable delivery catheter 209 within a host valvular structure 12 prior to treatment. Steering of the delivery assembly 200 is accomplished via manipulation of the pull-wire 260 as described herein, and permits the user to navigate the delivery assembly 200 through curved anatomy such as the aortic arch as shown in FIGS. 5 and 9A. Further, steering of the delivery assembly 200 assists the user in properly aligning the distal end of the steerable delivery catheter 209 and/or any component extendable therethrough, such as inner catheter 208, within the target site, e.g., the host valvular structure 12.

As mentioned above, when the pull-wire 260 of a steerable delivery catheter 209 is tensioned (e.g., pulled in the proximal direction), the distal segment 209′ of steerable delivery catheter 209 will bend in a planar manner, due to the aligned configuration of the slots 232 along the slotted tube 229 and the pull-wire lumen 218 aligned with the slot circumferential centers 238, extending in parallel to the central longitudinal axis A_{C}.

In some examples, the steerable delivery catheter may be torqued via rotation of the handle 204. If it is desired to bend or deflect the distal portion 209a of the steerable delivery catheter 209 in a different direction that that along which it bends when the pull-wire 260 is tensioned, the steerable delivery catheter 209 may be torqued or rotated (for example, rotated approximately 180° if the desired bending is in an opposite direction), and then the pull-wire 260 may be actuated (e.g., pulled) to bend the distal portion 209a of the steerable delivery catheter 209. Nevertheless, torquing (or rotating) of the handle, may only partially deflect the distal end (or distal portion 209a) of the steerable delivery catheter 209 relative to its state in the absence of wire-pulling, due to the radius of bending and the distance along which the distal end of the steerable delivery catheter 209 may be deflected. For example, torquing the handle may cause the distal end of the steerable delivery catheter 209 to move towards the vicinity of the central of aortic annulus 24 (such as the center of coaptation of the leaflets 30), as illustrated in FIG. 9B. While this centralized position may be sufficiently adequate for some interventional procedures, such as deployment of a prosthetic valve in conventional transcatheter aortic valve intervention (TAVI) procedures, a greater extent of deflection of the distal portion of the steerable delivery catheter 209, optionally across the entire width of the aortic root 22, may be required in some desired procedures, such as when treatment of a left leaflet 60, in the vicinity of the left coronary ostium 44, is desired.

According to examples of the current disclosure, a delivery apparatus 202 includes a steerable delivery catheter 210 configured to allow three-dimensional bending. Steerable delivery catheter 210 comprises a slotted tube 230 having a plurality of successive slots 232 which are circumferentially offset from each other. FIG. 10A shows a side view of an exemplary slotted tube 230, configured for out-of-plane articulation. FIG. 10B shows the slotted tube 230 of FIG. 10A in a flattened configuration, illustrating the manner by which the slotted tube is cut if the tube were to be longitudinally cut along its length to form slots 232 and laid flat. Slotted tube 230 can be similar to any example of slotted tube 229 described herein, except that at least some, and optionally all, of the slots 232 are circumferentially offset relative to adjacent slot(s) 232. For illustrative purposes, the slotted tube 230 is described herein, in some examples, as having three longitudinal portions, e.g., a distal portion 230a terminating at a tube distal end 266, an intermediate portion 230b which is proximal to the distal portion 230a, and a proximal portion 230c which is proximal to the intermediate portion 230b. The portions 230a, 230b, 230c are integral or continuous portions of the slotted tube 230 but may have, in some examples, different constructions as described herein. In some examples, the distal segment 210′ of steerable catheter 210 includes all three portions 230a, 230b and 230c of the slotted tube 230. In some examples, the distal segment 210′ of steerable catheter 210 includes the distal portion 230 of the slotted tube 230, while any of the intermediate 230b and proximal 230c portions can extend further along the intermediate segment 210″, an optionally further along at least part of the proximal segment 210′″ of catheter 210.

It is to be understood that while the steerable delivery catheter 210 can include segments or sub-segments which may not be steerable, as described above. The distal portion 210′ of steerable delivery catheter 210 is the portion of the catheter that includes the slotted tube 230 or at least a portion of the slotted tube 230, such that at least the distal segment 210′ is steerable. In some examples, the slotted portions 230a, 230b, and 230c are comprised in the distal segment 210′ of catheter 210. In some examples, the length LC1 of the distal segment 210′ of catheter 210′ (for example, see FIG. 4) is greater than the sum of the lengths L1+L2+L3 of the slotted portions 230a, 230b, and 230c of slotted tube 230 (for example, see FIG. 8A).

FIGS. 10C, 10D and 10E show a side view, a top view, and a bottom view, respectively, of a distal region of the slotted tube 230 of FIG. 10A, including the distal portion 230a and part of the intermediate portion 230b continuously extending therefrom. FIG. 10F shows the distal region of the slotted tube 230 of FIG. 10C in a flattened configuration, illustrating the manner by which the distal region of the slotted tube is cut if the tube were to be longitudinally cut along its length to form slots 232 and laid flat. The slotted tube 230 can be formed by seamless drawn tubing where slots are laser cut into the tube. The shape and size of each slot 232 can be generally similar to any example described for the shape and size of slots 232 above with respect to FIGS. 8A-8F.

Various exemplary implementations for delivery assemblies 200, delivery apparatuses 202, and/or steerable delivery catheter 210 thereof, can be referred to, throughout the specification, with superscripts, for ease of explanation of features that refer to such exemplary implementations. It is to be understood, however, that any reference to structural or functional features of any assembly, apparatus or component, without a superscript, refers to these features being commonly shared by all specific exemplary implementations that can be also indicated by superscripts. In contrast, features emphasized with respect to an exemplary implementation of any assembly, apparatus or component, including steerable delivery catheter 210 and/or a slotted tube 230 thereof, referred to with a superscript, may be optionally shared by some but not necessarily all other exemplary implementations. For example, slotted tube 230ª is an exemplary implementation of slotted tube 230, and thus includes the features described for slotted tube 230 throughout the current disclosure, except that while a slotted tube 230 can be generally provided with various angular and spatial configurations of circumferentially offset slots 232, slotted tube 230ª includes distal slots 232a, intermediate slots 232b and proximal slots 232c which are offset from their neighboring slots by the same offset angle.

In some examples, the slotted tube 230 further comprises a plurality of successive opposite cuts 244, as illustrated for example for slotted tube 230ª, the shape and size of which can be similar to any example described for the shape and size of opposite cuts 244 above with respect to FIGS. 8A-8F. In the example illustrated in FIG. 10B, the distal portion 230a of a slotted tube 230 includes a plurality of distal slots 232a, and optionally can further include a plurality of distal opposite cuts 244a. In some examples, the intermediate portion 230b of a slotted tube 230 can include a plurality of intermediate slots 232b, and optionally a plurality of intermediate opposite cuts 244b. In some examples, the proximal portion 230c of slotted tube 230 can include a plurality of proximal slots 232c, and optionally a plurality of proximal opposite cuts 244c. In some examples, the plurality of distal slots 232a can be equally spaced from each other along the longitudinal direction. In some examples, the plurality of intermediate slots 232b can be equally spaced from each other along the longitudinal direction. In some examples, the plurality of proximal slots 232c can be equally spaced from each other along the longitudinal direction. The opposite cuts 244 can be generally positioned midway between adjacent (or neighboring) slots 232 along the longitudinal direction, though other positions between adjacent slots 232 are contemplated.

In some examples, the slots 232 along the length of the slotted tube 230 are equally spaced from each other, such that the ribs 240 formed therebetween (for example, see FIG. 10D) have similar widths along the entire length of the slotted tube 230. In some examples, the slots 232 of at least one portion of the slotted tube 230 can be differently spaced from each other relative to the slots 232 of at least one other portion of the tube 230, such that the ribs 240 along different portions can be wider or narrower to change the radius of the greatest possible bend by the corresponding bending portions. In the illustrated example of slotted tube 230ª shown in FIG. 10B, the ribs 240 of the distal portion 230a are narrower than the ribs 240 of the intermediate portion 230b, and the ribs 240 of the intermediate portion 230b are narrower than the ribs 240 of the proximal portion 230c. This will result in variable flexibility of the slotted tube 230 such that the distal portion 230a is the most flexible.

In some examples, the plurality of distal slots 232a can be similarly shaped and dimensioned with respect to each other. In some examples, the plurality of intermediate slots 232b can be similarly shaped and dimensioned with respect to each other. In some examples, the plurality of proximal slots 232c can be similarly shaped and dimensioned with respect to each other. In some examples, the shape and dimensions of the slots 232 along the length of the slotted tube 230 are similar to each other. In some examples, the slots 232 of at least one portion of the slotted tube 230 can be differently shaped and/or dimensioned with respect to slots 232 of at least one other portion of the slotted tube 230, such that the different shapes and/or dimensions of the slots can change the radius of the greatest possible bend by the corresponding bending portions. In some examples, as illustrated in FIGS. 10A-10F, the distal slots 232a and intermediate slots 232b can be ovaloid-shaped slots, having a slot width W_{S} at their circumferential centers 238 greater than at their first and second ends 234, 236, while the proximal slots 232c are shown to have a uniform slot width W_{S}.

In some examples, the width W_{S} (for example, see FIG. 10C) and/or length L_{S} (for example, see FIG. 10F) of slots 232 along different portions of the slotted tube 230, or even between different slots 232 along the same portion of a slotted tube 230, can vary. Different dimensions of the slots 232 can allow for different articulation at different portions of the slotted tube 230, optionally creating a staged effect so that different sections can bend at different times. In some examples (not shown), the slot length L_{S} of the distal slots 232a is greater than the slots length L_{S} of the intermediate slots 232b and/or of the proximal slots 232c. In some examples, the slot length L_{S} of the intermediate slots 232b is greater than the slots length L_{S} of the proximal slots 232c. The decrease in slot length L_{S} in the proximal direction along the slotted tube 230 can allow the distal portion 210a of the steerable delivery catheter 210 to bend prior than the proximal portion 210c. By having longer distal slots 232a, which result in a smaller backbone 242 between the slot ends 234, 236, the distal portion 230a of the tube 230 will bend first as there is less material to bend.

In contrast to slotted tube 229, slots 232 of the steerable delivery catheter 230 are circumferentially offset from each other at an offset angle β (i.e., angle between axis A_{A} and axis A_{C}) as shown in FIG. 10B. In some examples, the slots 232 of steerable delivery catheter 230 are offset from each other. In some examples, at least the distal slots 232a of the distal portion 230a of steerable delivery catheter 230 are circumferentially offset from each other. FIGS. 10A-10F illustrate an angled axis A_{A}, which is angled at an offset angle β relative to the central longitudinal axis A_{C}. Any reference to slots 232 being circumferentially offset from each other, throughout the present disclosure and the claims, refers to the slot circumferential centers 238 of such slots 232 being aligned with the angled axis A_{A}, wherein the circumferential center 238 of each such slot 232 is circumferentially offset, by the offset angle β, from at least one adjacent slot 232, which can be a preceding slot and/or a succeeding slot. The angled axis A_{A} can be defined in a flattened configuration of the slotted tube 230, as shown in FIGS. 10B and 10F, defining the offset angle β with respect to the central longitudinal axis A_{C}, such that when the slotted tube 230 is in its tubular configuration (shown in FIGS. 10A and 10C-10E), the slot circumferential centers 238 follow a helical path around the central longitudinal axis A_{C}, such that the offset angle is the helix angle β of the helical path of the centers 238. This means that the backbone 242 of slotted tube 230 also forms a helix following helix angle β.

In some examples, the opposite cuts 244 of the slotted tube 230 can be similarly circumferentially offset from each other. If slots 232 of slotted tube 229 have identical slot lengths L_{S}, the slot first circumferential ends 234 can be similarly circumferentially offset from each other, and the slot second circumferential ends 236 can be similarly circumferentially offset from each other.

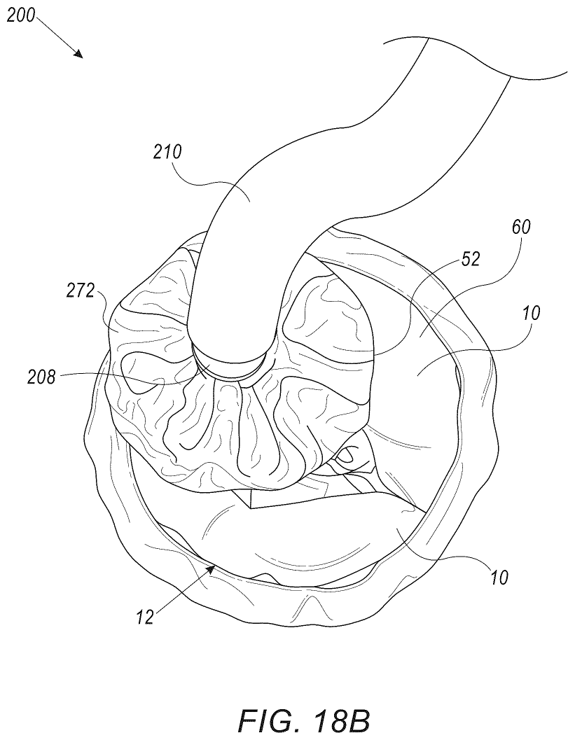

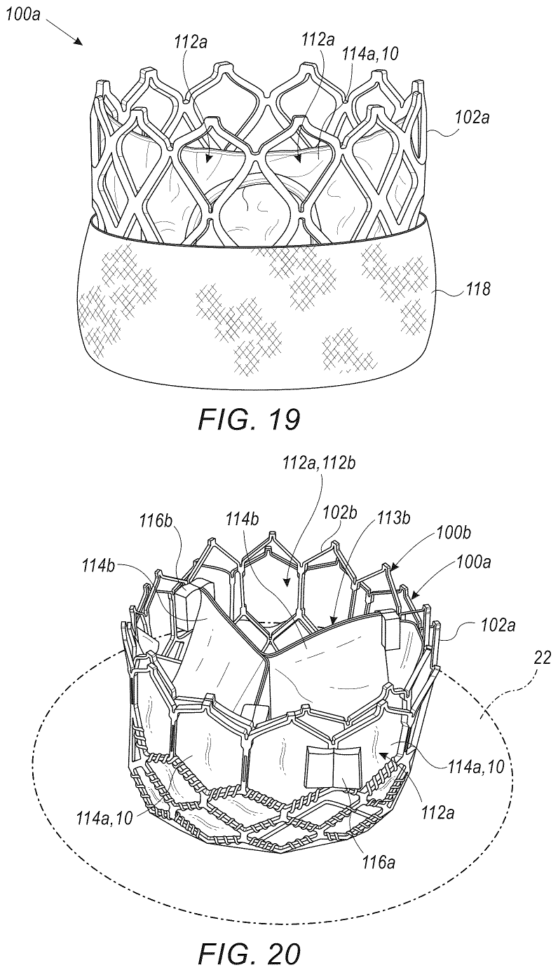

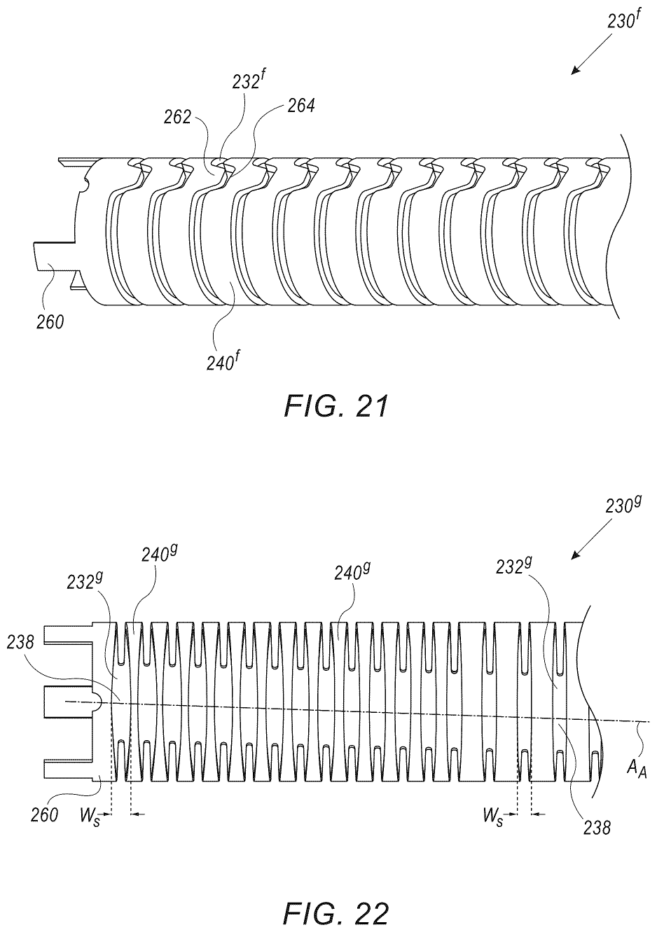

FIG. 23 shows a simplified view of a distal portion of a slotted tube 230 that includes two slots 232 which are circumferentially offset from each other by a circumferential offset angle α measured in a radial plane (i.e., a plane orthogonal to the central longitudinal axis A_{C}), while FIGS. 24-25 show a manner of deriving the helix angle β (in a flattened state of the tube, such as the state shown in FIG. 10F) from the circumferential offset angle α. The tube 230 and slots 232 are not drawn to scale but rather schematically shown to have simplified shapes and dimensions, with the angular offsetting of the slot circumferential centers 238 exaggerated for illustrative purpose only. FIG. 24 shows a simplified sectional view of the tube 230 of FIG. 23 along the radial plane. FIG. 25 shows a simplified flattened view of the tube 230 of FIG. 23. The axial distance between two adjacent slots 232 (such as between their respective circumferential centers 238) is marked by W_{C}, and R_{T} indicates the radius of slotted tube 230.

As shown in FIG. 24, the arch length between two adjacent slot circumferential centers 238 is α·R_{T}. When the tube 230 is flattened, as shown in FIG. 25, helix angle β is defined as an angle of a right triangle, with the arch length α·R_{T} being one leg of the triangle, opposite to the helix angle β, and the distance W_{C} between slots centers being the other leg perpendicular thereto. The helix angle β can be thus expressed as:

$$β = {tan}^{- 1} ( \frac{α \cdot {R}_{T}}{{W}_{C}} ) .$$

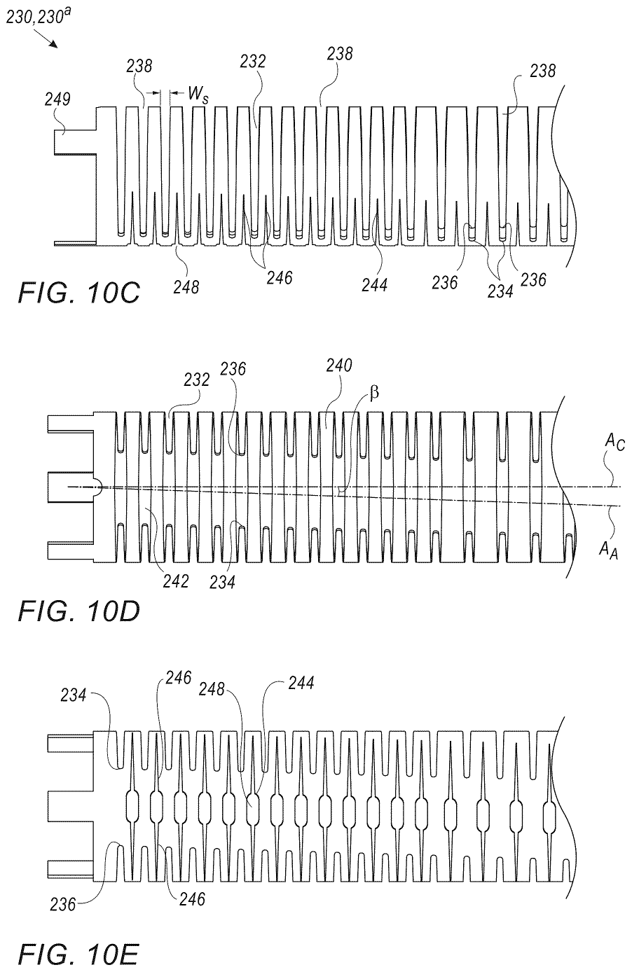

FIG. 11A show a top view of a distal region of an exemplary steerable delivery catheter 210 comprising a slotted tube 230, such as slotted tube 230^{a} illustrated in FIG. 10D, illustrating part of the distal segment 210′ of the catheter. FIG. 11B shows a perspective side view of the distal region of the steerable delivery catheter 210 of FIG. 11A. An optional braid 222 (for example, see FIG. 6) is not shown in any of the FIGS. 11A-11B, and the outer polymeric layer 224 is shown with partial transparency in FIG. 11A, and is removed from view in FIG. 11B, to show internal components of the steerable delivery catheter 210.

In some examples, the pull-wire lumen 218 of steerable delivery catheter 210, and the pull-wire 260 extending therethrough, is aligned with the slot circumferential centers 238, such that the pull-wire lumen 218 extends in a helical manner defining the helix angle β. This helical configuration of the slots 232 and the pull-wire lumen 218, helically extending around the central longitudinal axis A_{C}, will generally result in a three-dimensional out-of-plane movement of the catheter 210. The ribs 240 defined between successive slots 232 can be referred to as a series of hinged portions of the slotted tube 230, wherein the hinge points of the ribs 240 are distributed over the backbone 242 and slits 246 opposite to the slots 232. When the pull-wire 260 is proximally pulled, each rib 240 will move towards an adjacent rib in a manner that strives to close, or at least narrow, the gap defined by the slot 232 between such adjacent ribs 240, along a plane which is aligned with the slot circumferential center 238. If the slots circumferential centers 238 are circumferentially offset relative to each other, this will cause the corresponding ribs 240 to move close to each other along bending planes which are similarly offset from each other, such that the resulting bent geometry of the slotted tube 230 is three-dimensional.

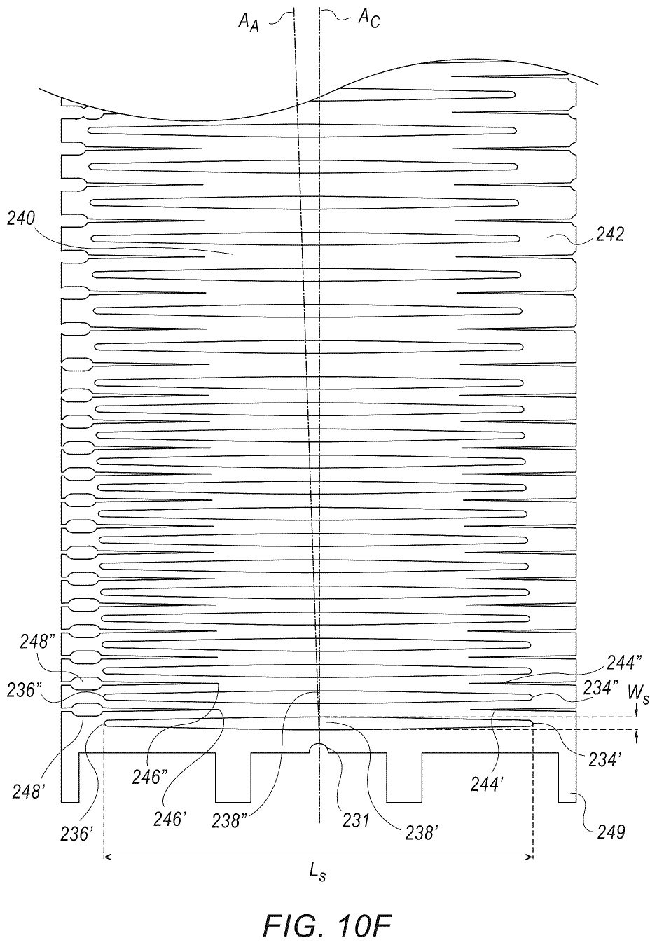

FIG. 12A shows a steerable delivery catheter 210 advanced, along the aortic arch 46, towards the native aortic valve 22. As described herein for steerable delivery catheter 209 with respect FIG. 9A, in some cases, such as when the catheter 210 is allowed to passively bend due to interaction with the native anatomical structures without being actively bent by pulling the pull-wire 260, as the steerable delivery catheter 210 is pressed against the aortic wall 38 during advancement through the curved aortic arch 46, the anatomical structure of the aortic arch 46 may orient the distal end of the steerable delivery catheter 210 towards a portion of the aortic root 22 adjacent to the right coronary ostium 44, such as towards the region of the right/non commissure 66 or other regions in the vicinity of the right leaflet 62 or non-coronary leaflet 64, which may be opposite to or different from a desired site of treatment in a procedure of modifying a leaflet (for examples, a left leaflet 60 to prevent obstruction of the left coronary ostium 42). Thus, it may be desired, in some occasions, to direct the distal end of the steerable delivery catheter 210, and any component extendable therethrough, such as an inner catheter 208, to a region that is different from the region at which the distal end of the steerable delivery catheter passively lands due to the native anatomy of the patient. In a typical aortic anatomy, the aorta is curved, such in a U-shaped geometry, along the aortic arch 46, and a portion of the ascending aorta 26 can be then curved at about a 30° angle relative to the plane defined by the aortic arch 46. Thus, the ascending aorta 26 can be referred to as a portion of the aorta bent out-of-plane, relative to the plane defined by the aortic arch 46. This three-dimensional out-of-plane geometry of the aorta, leading to the native aortic valve 20, makes it harder to navigate a distal portion of a catheter configured to bend along a two-dimensional plane, such as catheter 209, between different regions of the aorta.

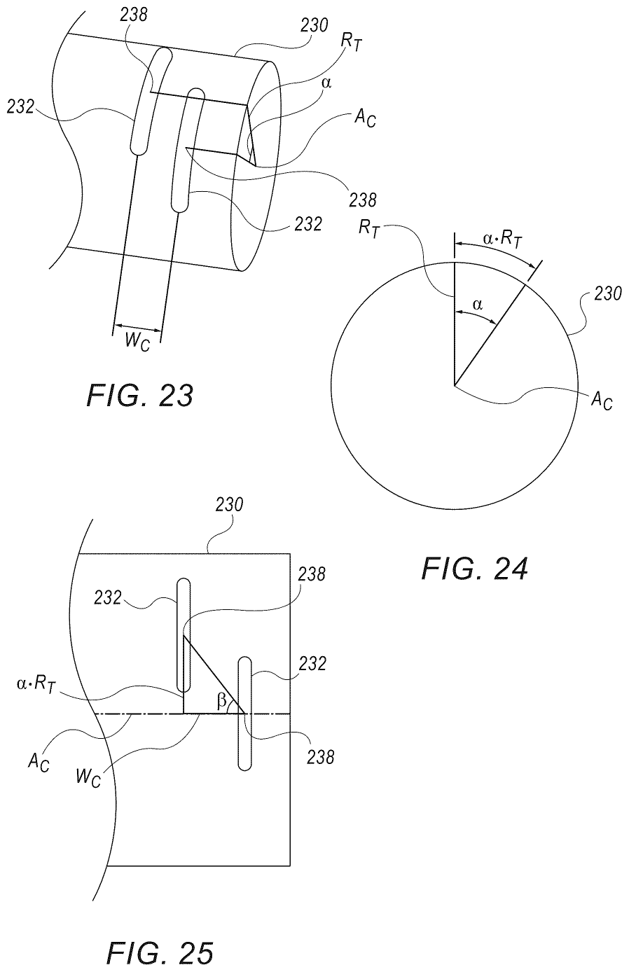

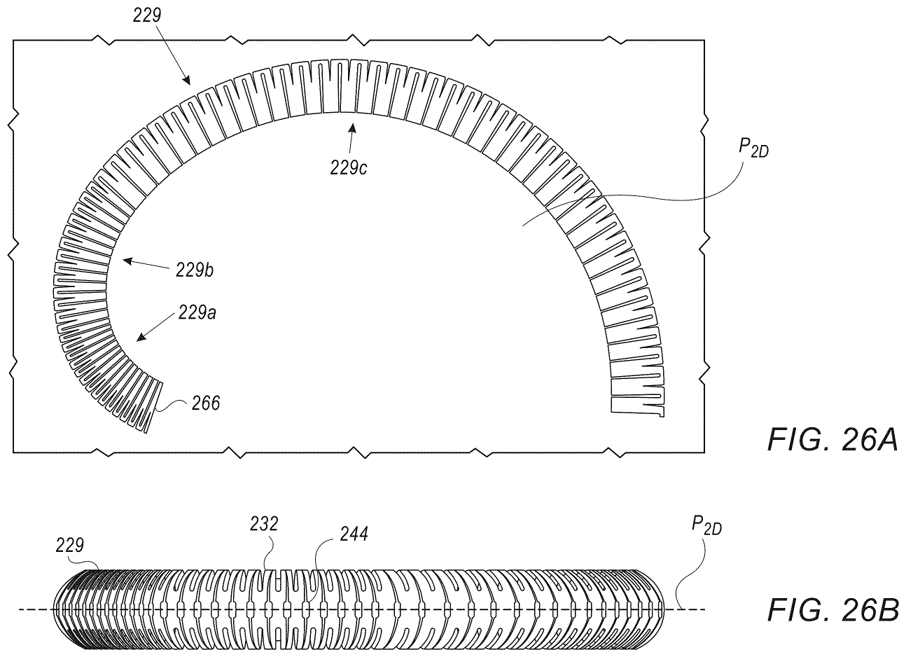

FIGS. 26A and 26B show a side view and a top view, respectively, of a slotted tube 229 curved in a two-dimensional plane P_{2D}, which is the plane of the drawing shown in FIG. 26A for example. This means that the central longitudinal axis A_{C} of the slotted tube 229 lies along the plane of the drawing sheet, without having any part thereof extending out of the plane defined by the drawing sheet. FIGS. 27A and 27B are top views of exemplary slotted tubes 230 curved in a three-dimensional or out-of-plane configuration, defined as a configuration in which the distal end of the tube 230, represented by the pull ring 250 for example, extends away from the two-dimensional plane P_{2D}) along which an equivalent slotted tube 229, similar in all respects except for being devoid of helical offset between slots thereof, would have been curved. A three-dimensional offset O_{L} can be defined as the distance of the tube distal end 266 from the two-dimensional plane P_{2D}, along a direction orthogonal to the two-dimensional plane P_{2D}). It is to be understood that any reference to a two-dimensional plane P_{2D} defined by a slotted tube 229 similarly refers to this plane being defined by a steerable tube 209 comprising the slotted tube 229, and that any reference to a three-dimensional geometry defines by a slotted tube 230 similarly refers to this geometry defined by a steerable tube 210 comprising the tube 230.

In some examples, a pull-wire 260 of a steerable catheter 210 can be pulled in a range between a minimal value configured to partially close the slots 232 such that a space still remains between edges of the slots 232, yet sufficient to assume an out-of-plane three-dimensional geometry of the catheter 210, and a maximal value in which the slots are further closed, optionally up to contact between adjacent edges of the slots (for example, fully closing the slot circumferential centers 238). In some examples, the handle 204 can include a knob or other adjustment mechanism (not shown) configured to allow an operator to pull the pull-wire 260 to a selected extent within such a range. The term “maximally pulled slotted tube 230” refers to a state in which the slots 232 are maximally closed within this range.

In some examples, FIG. 27A can be representative of a maximally pulled slotted tube 230 having a helix angle β of 0.5°, while FIG. 27B can be representative of a maximally pulled slotted tube 230 having a helix angle β of 1°, while other parameters that can influence the three-dimensional configuration, such as the axial distance between adjacent slots W_{C}, the tube radius R_{T}, and the like, can be similar for both tube examples. As shown for such examples, the greater the helix angle is, the greater is the three-dimensional offset O_{L} of the tube distal end 266 from the two-dimensional plane P_{2D}.

In some examples, FIG. 27A can be representative of a partially pulled slotted tube 230, while FIG. 27B can be representative of the same slotted tube 230 of FIG. 27A having its pull-wire 260 pulled to a greater extent, including being optionally maximally pulled in the state shown in FIG. 27B. As shown for such examples, when the pull-force applied to proximally pull or tension the pull-wire 260 is adjustable within a range, the operator can partially pull the pull-wire 260 to an extent that will result in a desirable three-dimensional offset O_{L} according to patient-specific anatomy or other factors, wherein the maximal offset distance O_{L} can be achieved when the pull-wire is maximally pulled.

As shown in the top view of FIG. 12B, when the pull-wire 260 of a steerable delivery catheter 210 is tensioned (for example, pulled towards a delivery handle), the distal portion 210′ of steerable delivery catheter 210 will bend out of plane, outside the two-dimensional plane described above with respect to FIG. 9B for example. In some examples, the helical pattern of slots 232, having their circumferential centers 238 angularly offset from each other at a helix angle β, as well as the optional similarly helical path of the pull-wire 260 when aligned with the slot circumferential centers 238, will cause the distal segment 210′ of steerable delivery catheter 210 to bend along the helical direction of the slots 232 with the enactment of the pull-wire 260 force, thus giving the delivery catheter 210 three-dimensional steerability. Depending on the helix angle β, along with the geometrical and spatial configuration of the slots, the extent to which the distal end of the delivery apparatus 202 articulates can be varied, optionally allowing it to bend across the aortic valve 20 towards the left leaflet 60, providing a bending geometry that better conforms to the out-of-plane anatomical geometry of the ascending aorta 26 relative to the aortic arch 46, as illustrated in FIG. 12B.

In some examples, slots 232 of a slotted tube 230 helically extend around the central longitudinal axis A_{C} at a helix angle β, while the pull-wire lumen 218 and/or pull-wire 260 extends parallel to the central longitudinal axis, such that the slot circumferential centers 238 of most or all of the slots 232 may be circumferentially offset from the pull-wire 260.

It may be desirable, in some examples, to avoid the helix formed by the slot circumferential centers 238 from completing more than one turn around the central longitudinal axis A_{C} along the length of the slotted tube 230. In some examples, the helix angle β is chosen to avoid the helix formed by the slot circumferential centers 238 from completing more than one turn around the central longitudinal axis A_{C} along the length the slotted tube 230. In some examples, the helix angle β is not greater than about 15°. In some examples, the helix angle β is not greater than about 10°. In some examples, the helix angle β is not greater than about 5°. In some examples, the helix angle β is between about 0.5° and about 10°. In some examples, the helix angle β is between about 1° and about 5°. In some examples, the helix angle β is between about 1.5° and about 4°.

An important advantage of the steerable delivery catheter 210 described herein is that it only requires a single pull-wire 260 to provide three-dimensional articulation for the distal segment 210′ of the catheter within a body lumen. As shown in FIG. 7C, the delivery catheter 210 can protrude radially into primary lumen 212, or be otherwise thicker, at the region through which the pull-wire 260 extends. Inclusion of a plurality of pull-wires can thus restrict the free space inside primary lumen 212, requiring the outer diameter of the catheter 210 to be larger to compensate for this effect. Thus, a single pull-wire 260 can advantageously occupy a smaller cross-sectional area. Moreover, utilizing only a single pull-wire 260 greatly simplifies the manufacturability as well as use of the steerable delivery catheter 210.

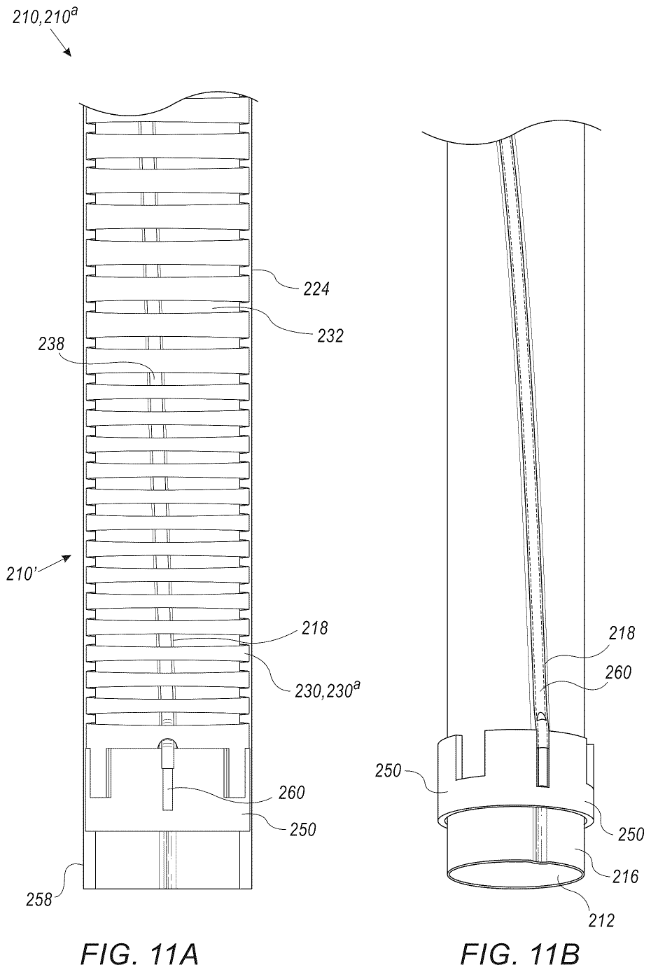

FIGS. 13A and 13B show a side view and a flattened view of an exemplary slotted tube 230b Slotted tube 230b is an exemplary implementation of slotted tube 230, and thus includes the features described for slotted tube 230 throughout the current disclosure, except that while slotted tube 230 can be provided with or without opposite cuts 244, slotted tube 230b is devoid of opposite cuts 244, such that the backbone 242 is formed as a fully uncut matter extending in a helical manner as illustrated.

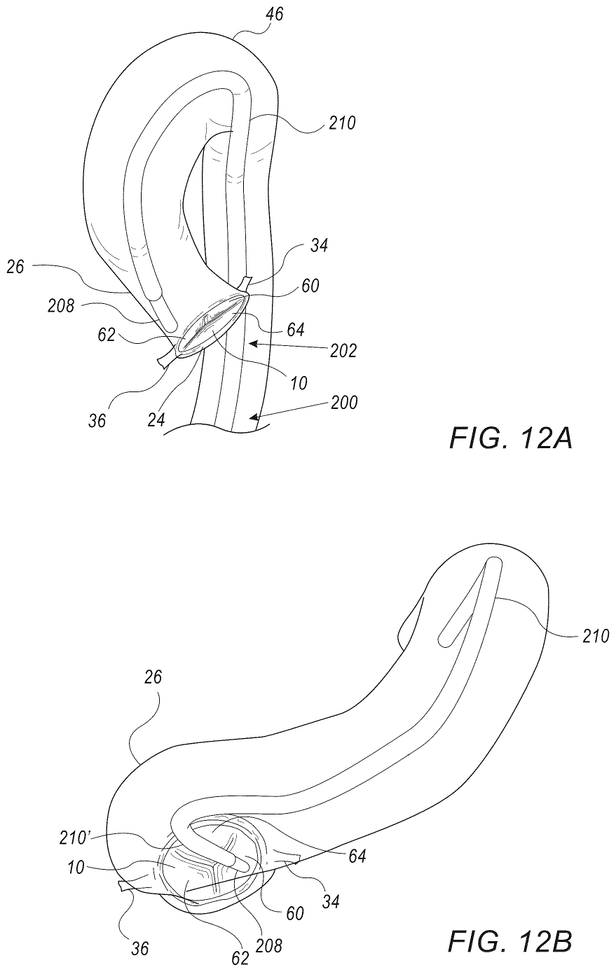

FIGS. 14A and 14B show a side view and a flattened view of a distal region of an exemplary slotted tube 230^{c}, respectively. Slotted tube 230^{c} is an exemplary implementation of slotted tube 230, and thus includes the features described for slotted tube 230 throughout the current disclosure, except that the slots 232 of the slotted tube 230^{c}, and optionally opposite cuts 244 when present, are orthogonal to the angled axis A_{A}, instead of being orthogonal to the central longitudinal axis A_{C} as illustrated for example in FIGS. 10A-10F.

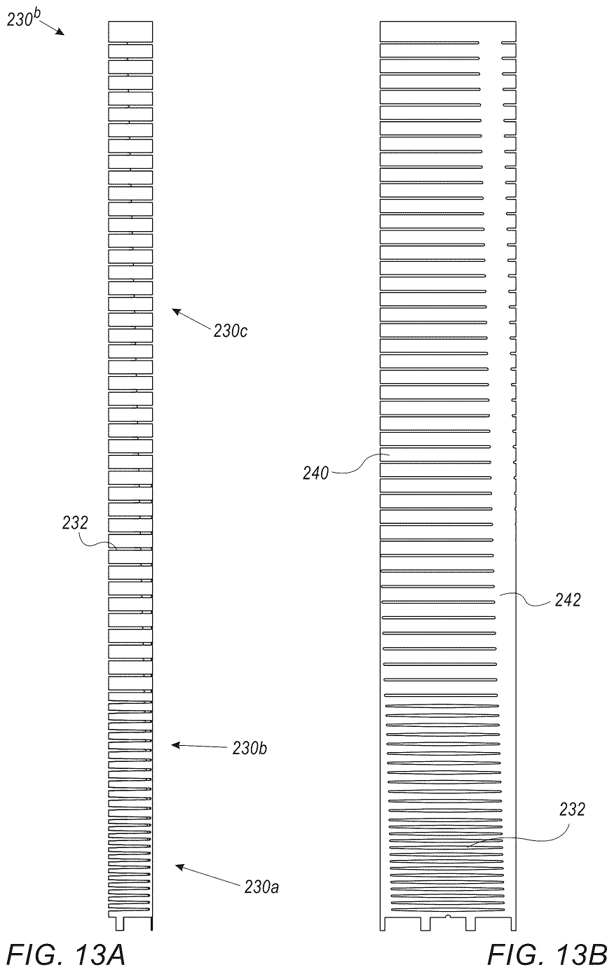

FIG. 15 shows a flattened view of an exemplary slotted tube 230d. Slotted tube 230d is an exemplary implementation of slotted tube 230, and thus includes the features described for slotted tube 230 throughout the current disclosure, except that different portions of slotted tube 230d can have a different helix angle β. For example, the distal portion 230a can have a distal helix angle βa, the intermediate portion can have an intermediate helix angle βb, and the proximal portion 230c can have a proximal helix angle βc, wherein at least two of the helix angles βa, βb, βc can be different from each other. The distal-most slot 232b of the intermediate portion 230b can be angularly offset from the proximal-most slot 232a of the distal portion 230a by any of the distal helix angle βa, the intermediate helix angle βb, or any angle therebetween. The distal-most slot 232c of the proximal portion 230c can be angularly offset from the proximal-most slot 232b of the intermediate portion 230b by any of the intermediate helix angle βb, the proximal helix angle βc, or any angle therebetween, but not by an angle that deviates from this range of helix angles.

In some examples, the distal helix angle βa is equal to the intermediate helix angle βb, but different from the proximal helix angle βc. In some examples, all three helix angles βa, βb and βc are different from each other. While the intermediate helix angle βb is shown to be greater than the distal helix angle βa in FIG. 15, and the proximal helix angle βc is shown to be greater than the intermediate helix angle βb, it is to be understood that in some example, the distal helix angle βa can be greater than the intermediate helix angle βb, and that in some examples, the intermediate helix angle βb can be greater than the proximal helix angle βc.

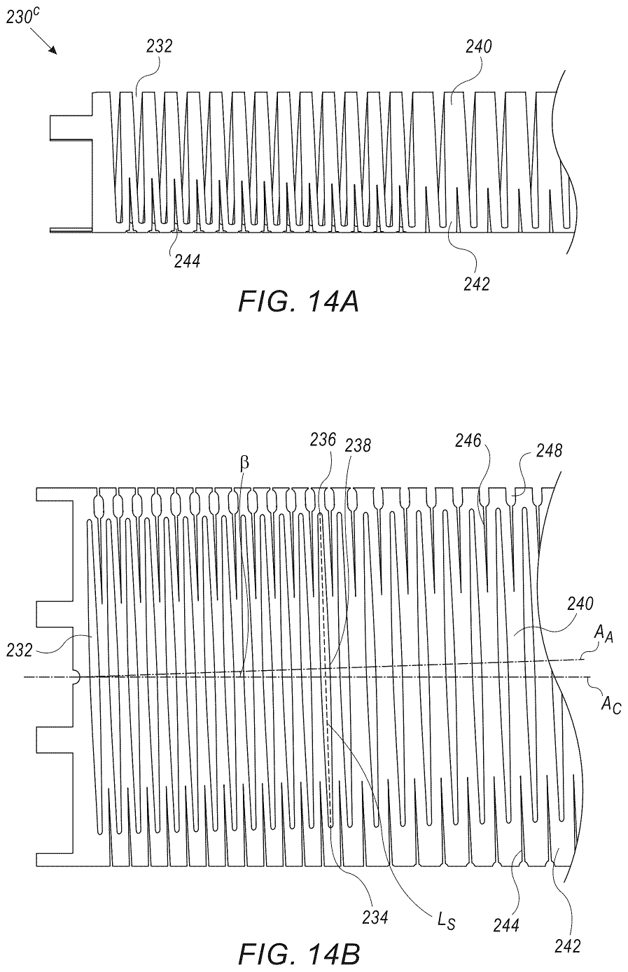

FIG. 16 shows a top view of a distal region of an exemplary steerable delivery catheter 210^{e}. Steerable delivery catheter 210e is an exemplary implementation of steerable delivery catheter 210, and thus includes the features described for steerable delivery catheter 210 throughout the current disclosure, except that the tip 258 of steerable delivery catheter 210^{e} can have a length L_{N} that is greater than the length L_{N} illustrated, for example, in FIG. 6. In some examples, the length L_{N} of the distal non-steerable portion 258 is greater than the diameter D_{L} of the primary lumen 212. In some examples, the length L_{N} of the distal non-steerable portion 258 is at least two times greater than the diameter D_{L} of the primary lumen 212.

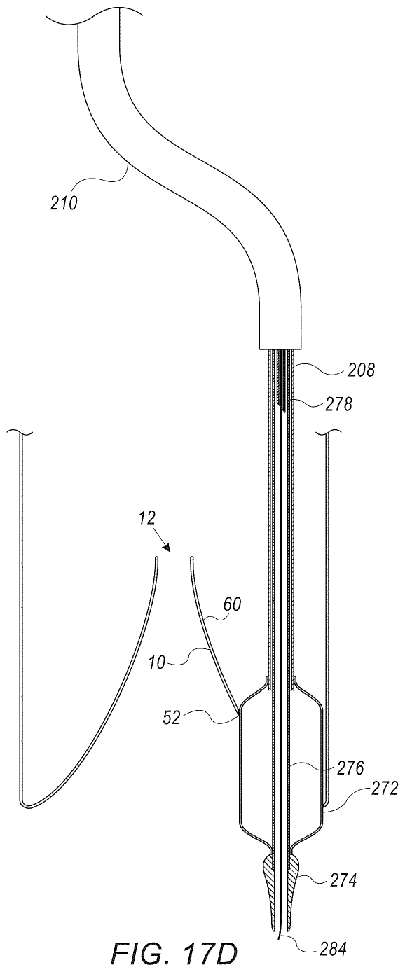

FIG. 21 shows a perspective view of an exemplary slotted tube 230^{f}. Slotted tube 230^{f} is an exemplary implementation of slotted tube 230, and thus includes the features described for slotted tube 230 throughout the current disclosure, except that one or more of the ribs 240^{b} includes a protrusion 262 extending towards a recess 264 of an adjacent rib 240^{b}. The protrusion 262 can generally extend in a direction perpendicular to the length of the slot 232, i.e., perpendicular to an imaginary line extending between the slot's first 234 and second 236 circumferential ends. In some examples, such as when the slots 232 are generally orthogonal to the central longitudinal axis A_{C}, as shown for example in FIGS. 10A-10F, the protrusion 262 can extend parallel to the central longitudinal axis A_{C}. In some examples, such as when the slots 232 are generally orthogonal to the angled axis A_{A}, as shown for example in FIGS. 14A-14B, the protrusion 262 can extend parallel to the angled axis A_{A}.

In some examples, the protrusions 262 can be located at the slots' circumferential centers 238. While the protrusions 262 are illustrated in FIG. 21 to be generally trapezoidal in shape, it is to be understood that other shapes are contemplated, such as curved or semi-circular protrusions, triangular protrusion, rectangular protrusion, and the like. While the protrusions 262 are shown in FIG. 21 to extend generally in a proximally-oriented direction, it is to be understood that in some examples, the protrusions 262 can extend in a generally distal direction, towards complementary recesses that can be formed along proximal edges of the ribs. The inclusion of protrusions 262 extending toward complementary recesses 264 improved the ability of the slotted tube 230^{f} to be torqued in clockwise or counterclockwise directions, as needed.

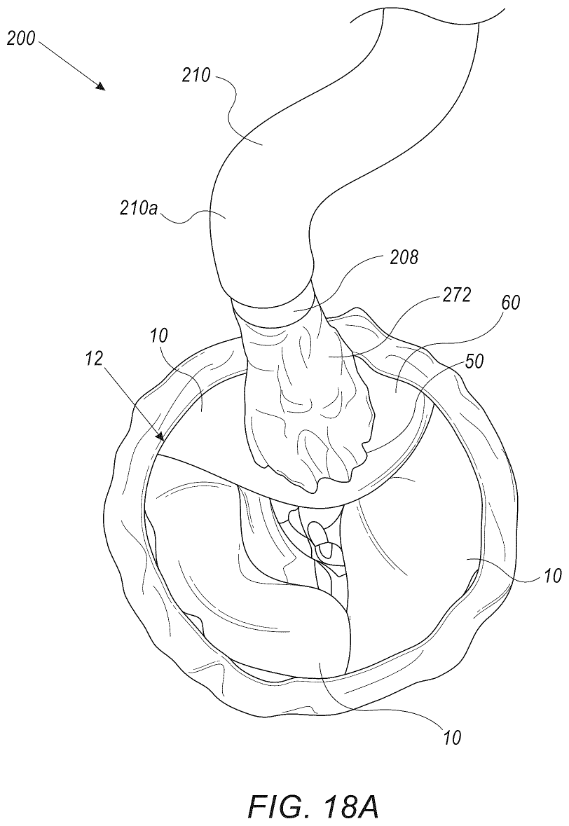

FIG. 22 shows a top view of an exemplary slotted tube 230^{g}. Slotted tube 230^{g} is an exemplary implementation of slotted tube 230, and thus includes the features described for slotted tube 230 throughout the current disclosure, except that at least some of the slots 232^{g} of slotted tube 230^{g} gradually change in width W_{S}, at least along their circumferential centers 238. In the illustrated example, each slot 232^{g} is shown to be wider, at least at its circumferential center 238, than a subsequent slot 232^{g} positioned proximal thereto. Gradually changing the widths W_{S} of the slots can vary the flexibility of the slotted tube 230^{g} along its length. Increasing the width W_{S} of the slots in the distal direction can increase the flexibility of the slotted tube 230^{g} towards the distal end.

FIGS. 17A-17D illustrate some steps in a method for utilizing a delivery assembly 200 in a method for forming a leaflet hole inside a host leaflet, which can be performed prior to implanting a guest prosthetic valve inside the host valvular structure. The delivery assembly 200 can be used to perforate, cut, and/or tear a host leaflet 10, such as a native leaflet 30 or a prosthetic valve leaflet 114 of a previously implanted prosthetic valve. The below method is an example of the manner by which the delivery assembly 200 that includes a delivery catheter 210 steerable in a three-dimensional space can be used. It will be understood that delivery assemblies 200 described herein can be used as part of other methods as well.

In some examples, the delivery apparatus 202 can further include a perforating device 270, configured to form an opening inside a host leaflet 10. The perforating device 270 can include a perforating member 278, which can be also referred to as a lacerating member 278, configured to form a pilot puncture 50 inside the host leaflet, and an expansion member 272 configured to be placed inside the pilot puncture 50, and to expand the pilot puncture 50 to form a larger leaflet opening 52.

The expansion member 272 may include and/or be any suitable structure for expanding the pilot puncture 50 to form the leaflet opening 52. In some examples, the expansion member 272 extends at least substantially around a circumference of the inner catheter 208 in order to exert an expansion force across full circumference of the pilot puncture 50. In some examples, the expansion member 272 may have a circular profile when in the radially expanded configuration. This is not required of all examples, however, and it additionally is within the scope of the present disclosure that the expansion member 272 may have a non-circular profile when in the radially expanded configuration.

In some examples, the expansion member 272 is an inflatable balloon 272 that can be mounted on a distal portion of a balloon catheter. In some examples, the inner catheter 208 extendable through the steerable delivery catheter 210 is the balloon catheter 208 over which an inflatable balloon 272 of the perforating device 270 is mounted. The balloon 272 is configured to transition between a deflated state and an inflated state.

The balloon catheter 208 can extend through the handle 204 and be fluidly connectable to a fluid source for inflating the balloon 272. The fluid source (not shown) comprises an inflation fluid. The term “inflation fluid”, as used herein, means a fluid (such as saline, though other liquids or gas can be used) used for inflating the balloon 272. The inflation fluid source is in fluid communication with a lumen of the balloon catheter 208, which in turn is in fluid communication with a cavity of the balloon 272, such that fluid from the fluid source can flow through the balloon catheter 208 to inflate the balloon 272. The balloon catheter 208 can be axially movable relative to the steerable delivery catheter 210. The delivery apparatus 202 can further include a dilator such as a nosecone 274, carried by a dilator shaft or nosecone shaft 276 extending through a lumen of the balloon catheter 208. The balloon catheter 208, the nosecone shaft 276, and the perforation member 278, can be configured to be axially movable relative to each other.

The proximal ends of the steerable delivery catheter 210, the balloon catheter 208, the perforating member 278, and optionally the nosecone shaft 276, can be coupled to the handle 204. During delivery of the assembly 200 and modification of a host valvular structure 12, the handle 204 can be maneuvered by an operator (e.g., a clinician or a surgeon) to axially advance or retract components of the delivery apparatus 202, such as the nosecone shaft 276, the balloon catheter 208, the steerable delivery catheter 210, and/or a perforating member 278, through the patient's vasculature and/or along the target site of treatment, as well as to inflate balloon 272 mounted on the balloon catheter 208, so as to enlarge a leaflet opening 52, and to deflate the balloon 272 and retract the delivery apparatus 202 when the procedure is completed.

The nosecone shaft 276 can extend through the lumen of balloon catheter 208 and define a lumen extending along the entire length of the nosecone shaft 276 and nosecone 274, through which a guidewire 284 can pass, such that the entire delivery apparatus 202 can be advanced toward the treatment region over the guidewire 284. In some examples, the perforating member 278 can extend through the balloon catheter 208, such as by extending through the lumen of balloon catheter 208 and/or a lumen of a shaft extending through balloon catheter 208, such as through a lumen of the nosecone shaft 276. In some examples, the perforating member 278 is axially movable relative to another component of the delivery apparatus 202, such as the balloon 272. In some examples, the perforating member 278 can extend through a lumen of nosecone shaft 276 and be axially movable relative to nosecone shaft 276. The perforating member 278 can define a lumen through which the guidewire 284 can extend.

The distal end portion of the delivery apparatus 202, including dilator or nosecone 274, is configured to be inserted into a patient's vasculature, such as within an ascending aorta 26, and to be advanced towards the host leaflet 10, wherein the guidewire 284 can optionally pierce through the host leaflet 10 as shown in FIG. 17A. Positioning the delivery apparatus 202 relative to the host leaflet 10 may comprise advancing the delivery apparatus 202 toward the leaflet via guidewire 284. In some examples, the delivery catheter 210 can be passively advanced to towards the aortic root 22 (i.e., allowed to passively bend while the pull-wire 260 remain unpulled). When the distal end portion of delivery apparatus 202 lands at the target site, the steerable delivery catheter 210 can be bent in an out-of-plane three-dimensional configuration, to orient the distal end portion of the delivery apparatus 202 towards the left leaflet 60, as described above with respect to FIGS. 12A-12B. In some examples, the pull-wire 260 can be proximally pulled to actively bend the delivery catheter 210 during advancement, such as during or prior to entry into the region of the aortic root 22, optionally during or prior to passage of the catheter 210 through the ascending aorta 26, allowing the distal portion of the catheter 210 to assume an out-of-plane three-dimensional configuration during advancement towards the aortic annulus 24 so that the distal end portion will be oriented towards the desired site of treatment (for example, the left leaflet 60) without contacting the aortic wall 38 at the opposite region, thereby avoiding undesirable friction of the catheter's distal portion against the aortic wall 38. In some examples, positioning the delivery apparatus 202 comprises positioning the perforating device 270 relative to the target host leaflet, which can comprise positioning a distal end of the inner catheter 208 in a pocket formed along a cusp edge of the host leaflet 10 and/or between a proximal surface of the host leaflet 10 and a portion of the host valvular structure 12 radially exterior of the leaflet 10.

While a steerable delivery catheter 210 is described and illustrated in some examples throughout the present specification as being used to bend towards a native left leaflet 60, it is to be understood that in the case of a procedure for modifying a leaflet of a previous implanted prosthetic valve, the steerable delivery catheter 210 can be similarly bent towards a desired prosthetic leaflet of the host valve, which is the leaflet closest to the left coronary ostium 42. Thus, any reference to maneuvering the steerable delivery catheter 210 to bend towards a left leaflet, a similar procedure may be performed with respect to a left native leaflet or a leaflet of a previously implanted prosthetic valve that is closest to the left coronary ostium.

As mentioned above, the delivery apparatus 202 can further include a perforating member 278, which can include a distal end portion 280 configured to pierce a host leaflet 10 of a host valvular structure 12 to form a pilot puncture 50 in the host leaflet 10, when a distal end portion 280 is positioned distal to the balloon 272.

In some examples, the distal end portion 280 of perforating member 278 is configured to be selectively translated in the proximal or distal directions relative to another component of the delivery apparatus 202, such as the balloon 272, balloon catheter 208, and/or the nosecone 274. In some examples, the nosecone shaft 276 and the perforating member 278 are configured to be movable axially relative to each other in the proximal and distal directions. The perforating member 278 can be coupled to a handle 204. The handle 204 can have one or more actuators (for example, in the form of rotatable knobs 206) that are operatively coupled to the perforating member 278 to facilitate axial movement thereof. In such examples, the distal end portion 280 can be configured to pierce a host leaflet 10 (such as left leaflet 60) when axially translated to a position which is distal to the balloon 272 and/or the nosecone 274. In some examples, the distal end portion 280 is not necessarily configured to be axially translatable relative to the balloon 272, in which case it is positioned distal to the balloon 272 at all times.

As shown in FIG. 17B, the perforating member 278 is configured to puncture the host leaflet 10 (such as left leaflet 60) to form a pilot puncture 50 within host leaflet 10. Subsequent to forming the pilot puncture 50, and as shown in FIG. 17C, the balloon 272 may be inserted within the pilot puncture 50. The distal end portion 280 of the perforating member 278 can be concealed within a lumen of the delivery apparatus 202, being positioned proximal to a distal end of the nosecone 274 during delivery, as shown in FIG. 17A, to avoid damage that may be caused to internal anatomical structures of the patient's body due to accidental contact with an optionally sharp distal end portion 280. The distal end portion 280 can be then pushed toward and through the host leaflet 10 to form the pilot puncture 50 as shown in FIG. 17B. The distal end portion 280 can be retracted back to re-conceal the distal end portion 280.

In some examples, the perforating member 278 can be retracted so as to position the distal end portion 280 proximal to the distal end of the nosecone 274 to place balloon 272 inside the pilot puncture 50, relying on the distal end of the nosecone 274 being small enough to allow insertion of the tapering nosecone 274 through pilot puncture 50. In some examples, the distal end portion 280 of the perforating member 278 remains distal to the distal end of the nosecone 274 during advancement of the nosecone 274 and the balloon 272 through the pilot puncture 50. In some examples, the distal end portion 280 of the perforating member 278 is retracted to conceal the perforating member 278, positioning its distal end portion 280 proximal to the distal end of the nosecone 274, as illustrated in FIG. 17C.

With the balloon 272 received within the pilot puncture 50, inflating the balloon 272 to transition it from the radially deflated state (FIG. 17C) to the radially inflated state (FIG. 17D) can expand the pilot puncture 50 to form a leaflet opening 52 that is sized to receive a guest prosthetic valve 100 in a radially compressed or crimped configuration thereof.

In some examples, inflating the balloon 272 within the host leaflet 10 (such as left leaflet 60) serves to increase a diameter of the pilot puncture 50 such that the resulting leaflet opening 52 is a hole with an increased diameter relative to the pilot puncture 50. In some examples in which the leaflet opening 52 is a hole, the leaflet opening 52 may be a substantially circular hole. In other examples, the leaflet opening 52 may be non-circular (for example, elliptical or asymmetric). In such examples, the diameter of the leaflet opening 52 may refer to any suitable dimension of the leaflet opening 52, such as a minimum diameter of the leaflet opening 52, a maximum diameter of the leaflet opening 52, and/or an average diameter of the leaflet opening 52.

In some examples, inflating the balloon 272 within the host leaflet 10 may cause the host leaflet 10 (such as left leaflet 60) to rip and/or tear such that the leaflet opening 52 is not a bounded hole. Stated differently, in such examples, the leaflet opening 52 may be formed by a tear that extends from the pilot puncture 50 fully to the free edge of the host leaflet 10 (the coaptation edge of the leaflet).

The delivery apparatus 202 may be configured to form the leaflet opening 52 in any of a variety of host valvular structures 12. In the example of FIGS. 17A-17D, the host valvular structure 12 can be the valvular structure 113 of a previously implanted prosthetic valve, such as the prosthetic valve 100a of FIG. 3. In such examples, using the delivery assembly 200 as described herein to form the leaflet opening 52 in a previously implanted prosthetic valve may be followed by steps for implanting a guest prosthetic valve 100b within the previously implanted prosthetic valve 100a (for example, via a ViV procedure).

Similarly, the host valvular structure 12 in the example of FIGS. 17A-17D can be a valvular structure 29 of a native heart valve, such as the native aortic valve 20 shown in FIGS. 2A-2B. In such examples, the perforating member 278 can be configured to puncture a native leaflet 30 (such as the left leaflet 60) of the native aortic valve 20. In other examples, the host valvular structure and/or the native valve may refer to another valve of a patient's heart, such as a mitral valve, a pulmonary valve, or a tricuspid valve.

In some examples, the perforating member 278 may include and/or be a needle, such as a spring-loaded needle and/or a Veress needle. As shown in FIGS. 17A-17D, the distal end portion 280 of the perforating member 278 can terminate at an angled surface 282. The angled surface 282 can have a sharp cutting edge to facilitate piercing the host leaflet 10 when the needle is pressed against the leaflet.

As further mentioned above, in some examples, the perforating member 278 can define a lumen configured to accommodate a guidewire 284 that can extend therethrough. In such examples, the guidewire 284 can be inserted into the patient's vasculature, and then the perforating member 278 and/or other shafts of the delivery apparatus 202 may be advanced toward the host leaflet 10 over the guidewire 284.

In some examples, the guidewire 284 can be used as a perforating or lacerating member for forming a pilot puncture 50. In such examples, the guidewire 284 can be a relatively stiff wire having a distal tip 286 configured to pierce the host leaflet 10 when the guidewire 284 is pressed against the leaflet. In some examples, the guidewire 284 can include a radio-frequency (RF) energy delivery tip 286 to assist with penetration through the leaflet tissue. For this purpose, a suitable RF energy device may be coupled to the guidewire 284, and the RF energy device can apply the RF energy to the guidewire tip 286 to penetrate the host leaflet 10. In any examples disclosed herein wherein a guidewire is used to puncture a leaflet, the guidewire can be coupled to a source of RF energy that applies RF energy to the tip of the guidewire. When the guidewire 284 is used to pierce the leaflet 10, the perforating member 278 in the form of a needle can be omitted, or it can be used in combination with the guidewire 284 that forms an initial puncture in the leaflet 10. For example, the guidewire 284 can be used to form an initial pilot puncture 50 (see FIG. 17A). In some examples, the perforating member 278 can be advanced through the leaflet to form a slightly larger pilot puncture (see FIG. 17B) for subsequent advancement of the balloon 272 through the host leaflet 10.

In some examples, the guidewire 284 is used as a perforating member without any additional separate perforating member, such as a needle, disposed thereover, such that the guidewire 284 can be utilized as the sole component that forms the pilot puncture 50, allowing other components of the delivery apparatus, such as dilator or nosecone 274, to pass through the pilot puncture 50, in a manner similar to that illustrated in FIG. 17B, but without the needle 278.

In some examples, the guidewire 284 is used as a perforating member that can be used in addition to perforating member (e.g., needle) 278, such that the guidewire 284 can form an initial puncture via a sharp tip 286 or an RF energy delivery tip 286, as illustrated in FIG. 17A, followed by penetration of the perforating member 278 into the leaflet 10 to form the pilot puncture 50, or a pilot puncture 50 which is greater in size than an initial puncture formed by the guidewire tip 286, as shown in FIG. 17B.

In some examples, the guidewire tip 286 is not necessarily sharp enough or otherwise configured to puncture through the host leaflet 10, in which case the guidewire 284 can be utilized for advancement of the delivery apparatus 202 toward the valvular structure 12, but terminate in proximity of the host leaflet 10 without piercing through it (for example, remaining above host leaflet 10 instead of passing through the tissue as shown in FIG. 17A), and the distal end portion 280 of perforating member 278 can be then advanced towards and into the host leaflet 10, to form the pilot puncture 50 in a similar manner to that illustrated in FIG. 17B.

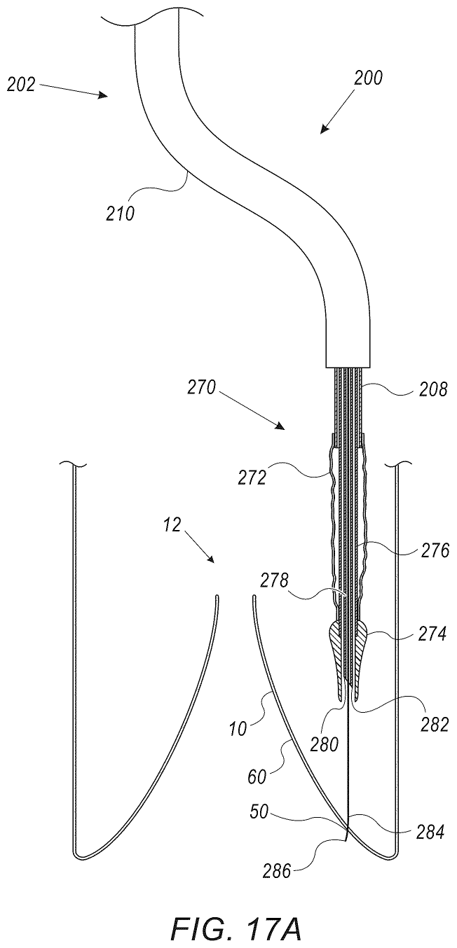

FIGS. 18A-18B illustrate the balloon 272 utilized to expand the pilot puncture 50 into the leaflet opening 52. FIG. 18A illustrates the balloon 272 in the deflated state within the pilot puncture 50, corresponding to the state described above with respect to FIG. 17C, while FIG. 18B illustrates the balloon 272 in the inflated state such that the pilot puncture 50 has enlarged into the leaflet opening 52, corresponding to the state described above with respect to FIG. 17D.

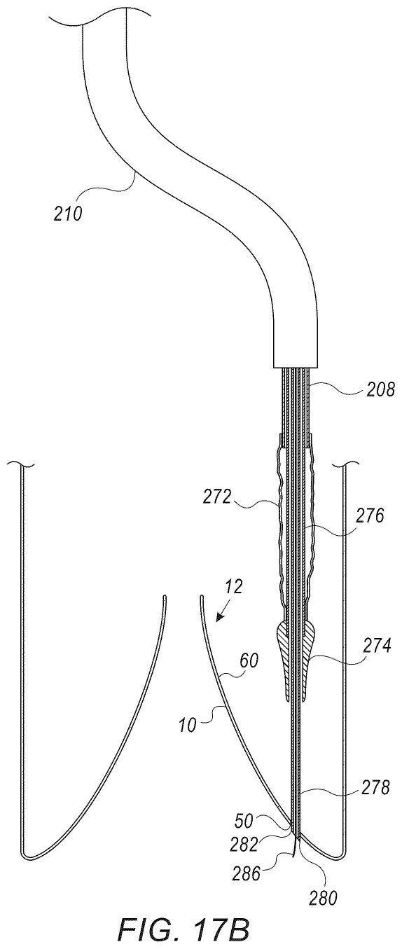

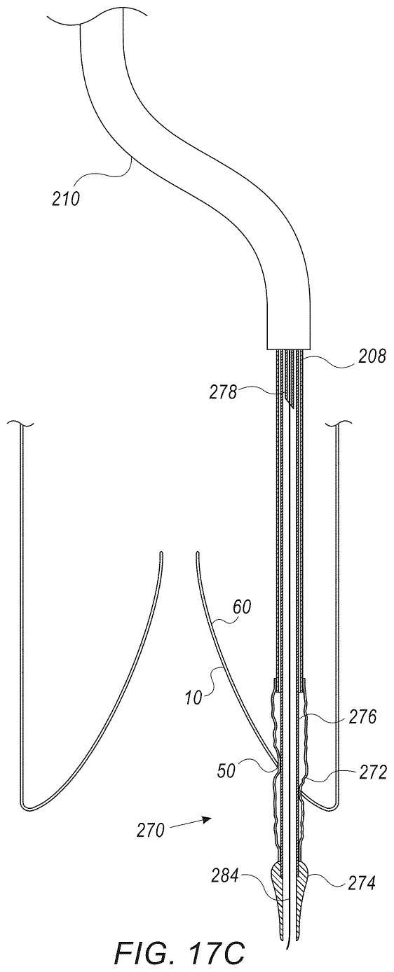

As mentioned, the delivery assemblies and methods of the current specification can be utilized for forming a leaflet opening 52 in a host leaflet 10 which can be either a native leaflet 30 or a prosthetic valve leaflet 114 of a previously implanted prosthetic valve, such as prosthetic valve 100a of FIG. 3, for example in the case of ViV procedures. FIG. 19 shows a previously implanted prosthetic valve 100a subsequent to forming the leaflet opening 52, for example subsequent to the method described above with respect to FIGS. 17A-18B or any equivalent thereof. FIG. 20 shows a configuration in which a second prosthetic valve 100b has been expanded within the leaflet opening 52 of a host prosthetic valve 100a. In the example of FIG. 20, the guest prosthetic valve 100b is the same type of valve as the host prosthetic valve 100a. It is to be understood, however, that the methods described herein, when implemented in ViV procedures, also may be applied to any other suitable valvular structures, such as different prosthetic valves and/or native heart valves. For example, the guest prosthetic valve 100b need not be the same type of valve as the host prosthetic valve 100a.

In the example of FIG. 19, when the prosthetic valve leaflets 114a of the previously implanted prosthetic valve 100a are pressed against the frame 102a, the leaflet opening 52 provides a partial access into the frame 102a, but the leaflet opening 52 may not be sufficiently large to completely uncover any of the cell openings 112a of the frame 102a.

As shown in FIG. 20, however, fully expanding the guest prosthetic valve 100b within the leaflet opening 52 further expands and/or tears the leaflet opening 52 such that several cell openings 112a of the frame 102a of the host prosthetic valve 100a and several cell openings 112b of the frame 102b of the guest prosthetic valve 100b are fully uncovered by the leaflets 114a. In some examples, this may result from the frame 102b of the guest prosthetic valve 100b pushing the leaflet 114a comprising the leaflet opening 52 downwardly (toward the inflow ends of the prosthetic valves 100a, 100b) such that one or more cell openings 112a are unobstructed by the leaflet 114a. In some examples, expanding the frame 102b within the leaflet 114a comprising the leaflet opening 52 may rip and/or tear this leaflet 114a such that the leaflet 114a cannot obstruct one or more cell openings 112a.

While the methods disclosed herein refer to forming a leaflet opening 52 in a host leaflet 10, prior to positioning and expanding a prosthetic valve 100, it is to be understood that any of the methods can comprise, in some examples, repeating one or more steps disclosed throughout the current specification to form a plurality of punctures and openings in the host valvular structure. For example, steps described above with respect to FIGS. 17A-18B and equivalents thereof, can be performed for forming a first leaflet opening in a first host leaflet. In some examples, the balloon can be deflated, and the delivery apparatus can be retracted from the first host leaflet and steered, utilizing the steering mechanism of steerable delivery catheter 210, toward another host leaflet. In some examples, the same steps can be repeated to form a second leaflet opening within the second host leaflet. The procedure can be optionally repeated to form further leaflet openings, such as a third leaflet opening in a third host leaflet.

In some examples, forming more than one leaflet opening, such as forming the second leaflet opening, can provide further access and/or fluid paths through the frame of the guest prosthetic valve. For example, radially expanding the guest prosthetic valve 100 within the first leaflet opening may push the second host leaflet against the frame of the guest prosthetic valve such that the second leaflet opening is aligned with cell opening(s) of the frame of the guest prosthetic valve. Thus, the second leaflet opening can provide additional unobstructed paths through the frame of the guest prosthetic valve. Moreover, in an example in which the host valve is a previously implanted prosthetic valve, expanding the guest prosthetic valve within the first leaflet opening can trap the second leaflet opening between the respective frames of the host prosthetic valve and the guest prosthetic valve, thereby providing additional access and/or flow paths through each of the frames.

Thus, forming the second leaflet opening can ensure that a greater number of cell openings of the frame are uncovered, and/or that a greater proportion of the frame is uncovered, relative to an example in which only one leaflet is punctured to form a leaflet opening. This may be particularly beneficial in examples in which the frame of a host prosthetic valve extends axially in a downstream direction beyond one or both of the coronary arteries when the guest prosthetic valve is implanted within a native heart valve.

Specifically, in some patient anatomies, the left coronary artery 34 is positioned lower (that is, proximate to the host valvular structure) than the right coronary artery 36. In such examples, the right coronary artery 36 may be sufficiently far from the host valvular structure that implanting the guest prosthetic heart valve within the host valvular structure does not limit access and/or perfusion to the right coronary artery 36. Accordingly, forming a single leaflet opening in the host valvular structure may be sufficient to ensure access and/or perfusion to both coronary arteries, provided that the leaflet opening is formed and/or positioned to ensure access to the left coronary artery 34.

In other examples, however, each of the left and right coronary arteries may be positioned sufficiently proximate to the host valvular structure, such that forming a single leaflet opening in the host valvular structure is insufficient to ensure access to both coronary arteries. In such examples, forming two leaflet openings in respective leaflets of the previously implanted prosthetic heart valve may ensure the ability for future access into both coronary arteries or perfusion through the frame to both coronary arteries during the diastole phase of the cardiac cycle. As a more specific example, the host valvular structure can be modified such that the guest prosthetic valve is implanted by being expanded in a leaflet opening of a first host leaflet (for example, left leaflet 60) that faces the left coronary artery 34, and such that the second leaflet opening is formed in a second host leaflet (for example, right leaflet 62) that faces the right coronary artery 36 (or vice-versa).

In some examples, forming the first leaflet opening can be performed prior to forming the second leaflet opening. In other examples, forming the second leaflet opening can be performed prior to forming the first leaflet opening. In some examples, the order of forming leaflet openings is chosen such that the final leaflet opening is formed in the host leaflet in which the prosthetic valve 100 is to be positioned and expanded.

It is to be understood that the guest prosthetic valve 100 is not limited to being implanted within an opening 52 of a leaflet. For example, in cases where the balloon 272 forms a full tear in a host leaflet that extends to the coaptation edge of the leaflet, the guest prosthetic valve 100 can be positioned at a location between the leaflets of the host valvular structure, for example by retracting the delivery apparatus from the host leaflet in which a leaflet opening is formed, repositioning by optionally utilizing steerable delivery catheter 210, and readvancing it such that the prosthetic valve 100 is positioned between the host leaflets, and then expanding the prosthetic valve 100. In some examples, such as in cases where the opening 52 does not form a full tear in the leaflet, the guest prosthetic valve can be positioned at a location between the leaflets of the host valvular structure 12 (such that the delivery assembly 200 used to implant to guest prosthetic valve 100 does not extend through the leaflet opening 52) and then expanded. In such cases, the opening 52 may provide sufficient open space through which blood may flow into the coronary ostia, and/or through which additional access devices, such as coronary catheters, can pass during future interventional procedures.

While described as an inflatable balloon in some examples above, it is to be understood that the expansion member 272 can be implemented, in some examples, to include non-inflatable expansion mechanisms. In some examples, the expansion member 272 can comprise and/or be an expandable frame. Some examples of expandable frames that can be utilized for expanding a pilot puncture 50 to form a leaflet opening 52 are described in U.S. Provisional Application No. 63/355,739, which is incorporated herein by reference in its entirety.

In some examples, one or more steps of the method described above with respect to FIGS. 17A-18B may be performed and/or facilitated with one or more guidewires. For example, positioning the perforating device 270 relative to the host leaflet 10 may comprise advancing and steering the delivery assembly 200 toward the host leaflet 10 via a perforating device guidewire, such as the guidewire 284 disclosed herein. In some examples, positioning a guest prosthetic valve within the host valvular structure 12, can comprise advancing the guest prosthetic valve toward the host valvular structure 12, and optionally into the leaflet opening 52, via a guest prosthetic valve guidewire. In some examples, the guest prosthetic valve guidewire and the perforating device guidewire refer to the same guidewire. In some examples, the perforating device guidewire extends alongside the guest prosthetic valve guidewire.

Radially expanding a guest prosthetic valve inside the host valvular structure 12, and optionally inside the leaflet opening 52, may be performed in any suitable manner, such as using any suitable valve expansion technique and/or mechanism that is known to the art. In some examples, radially expanding a guest prosthetic valve can comprise inflating an inflatable balloon on which the guest prosthetic valve is mounted.

In some examples, the delivery assembly 200 comprises a guest prosthetic valve 100 that can be carried, in a crimped configuration, towards the site of implantation, over a component of the delivery apparatus 202. In some examples, the delivery apparatus 202 comprises both the perforating device 270 and a catheter for delivering and expanding a guest prosthetic valve 100. In some examples, the delivery apparatus 202 comprises two separate inflatable balloon, a first inflatable balloon 272 for expanding a pilot puncture 50 to form a leaflet opening 52 as described above, and a second balloon (not shown) that can be inflatable to a greater diameter, for expanding a balloon-expandable prosthetic valve. Examples of delivery assemblies that include two separate balloons, one for forming a leaflet opening, and one for expanding a guest prosthetic valve, are described in U.S. Provisional Application Nos. 63/447,453 and 63/447,457, each of which is incorporated herein by reference in its entirety.

In some examples, the guest prosthetic valve can be a mechanically expandable prosthetic valve, and radially expanding the guest prosthetic valve can comprise actuating a mechanical actuator of the guest prosthetic valve to mechanically expand a frame of the guest prosthetic valve.

In some examples, the guest prosthetic valve can be a self-expandable prosthetic valve. When the guest valve is a self-expandable prosthetic valve, the act of radially expanding the guest valve can comprise advancing the guest valve from a delivery capsule or otherwise removing a restraint from the guest valve (which can be a distal portion of the inner catheter 208) to allow it to radially self-expand within the leaflet opening 52 (or another region within host valvular structure 12).

In some examples, when a guest prosthetic valve is received within the leaflet opening 52, radially expanding the guest prosthetic valve can serve to increase a size of the leaflet opening 52 and/or to tear the host leaflet 10. As a result, and as discussed above, radially expanding a guest prosthetic valve can serve to modify the host leaflet 10 such that the leaflet 10 does not obstruct a cell opening 112 in a frame 102 of the gust prosthetic valve 100 or at least increases the area of the host valve 100a and the guest valve 100b that is not covered or obstructed by the modified leaflet 10 to permit access and sufficient perfusion to the adjacent coronary artery. For example, radially expanding the guest prosthetic valve within the leaflet opening 52 can operate to push a portion of the leaflet 10 extending radially exterior of the guest prosthetic valve below an upper edge of an outer skirt 118 of the guest prosthetic valve and/or away from one or more cell openings 112 of the guest prosthetic valve.

Any of the assemblies, devices, apparatuses, etc. herein can be sterilized (for example, with heat, radiation, and/or chemicals, etc.) to ensure they are safe for use with patients, and any of the methods herein can include sterilization of the associated assembly, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

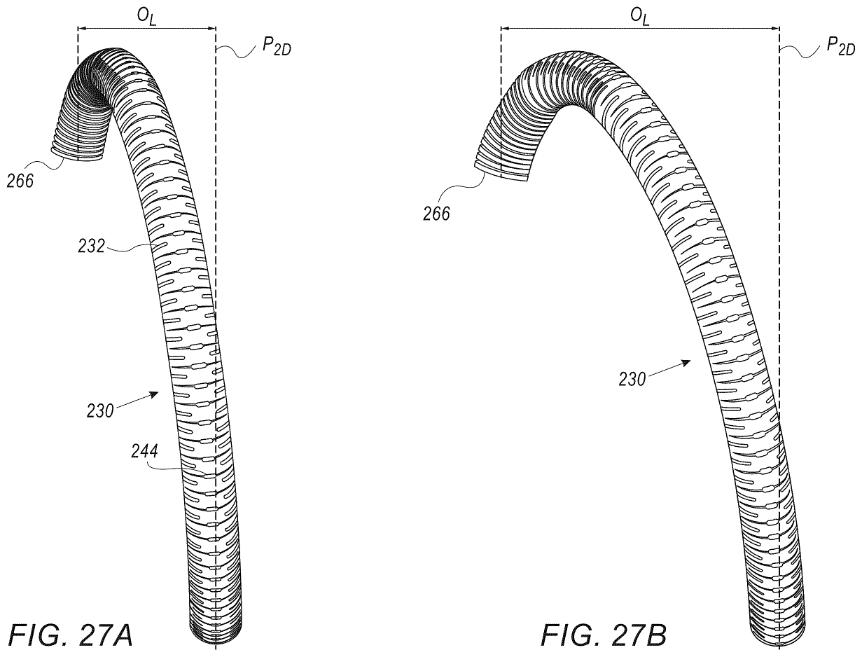

Reference is now made to FIGS. 28-30C, which illustrate a method for modifying a steerable delivery catheter, in some examples.

In some examples, herein a steerable delivery catheter 209 is described with a slotted tube 229, which has plurality of slots 232 circumferentially aligned with each other along the length of the slotted tube 229. Such slotted tube 229 is presented e.g., in FIGS. 8A-F. Further presented herein is a steerable delivery catheter 210 with a slotted tube 230, which has plurality of slots 232 with circumferential centers 238 circumferentially offset from the circumferential center 238 of the adjacent slot by a helix angle. Such slotted tube 230 is presented e.g., in FIGS. 10A-F with corresponding delivery catheter 210 in FIGS. 11A-B.

In some examples, the present method generally aims to modify a steerable delivery catheter 209 with slots 232 circumferentially aligned with each other, into a delivery catheter 210 with adjacent slot circumferential centers 238 offset by a helix angle. As will be described below, the method may further include, in some examples, synchronically twisting the polymeric layer 215, the pull-wire lumen 218 and/or the braid 222 with slotted tube radially to have a similar angle.

Thus, in some examples, the terms “modifying” and “modification” in the context of the present method are interpreted generally as forming or changing a helical angle between two or more of the slot circumferential centers 238 of the slotted tube 229. In some examples, the modification comprises forming the helical angle between two or more of the slot circumferential centers 238 of the slotted tube 229. In some examples, the modification comprises forming or changing a helical angle between two or more adjacent slot circumferential centers 238 of the slotted tube 229. In some examples, the modification comprises forming a helical angle between two or more adjacent slot circumferential centers 238 of the slotted tube 229.

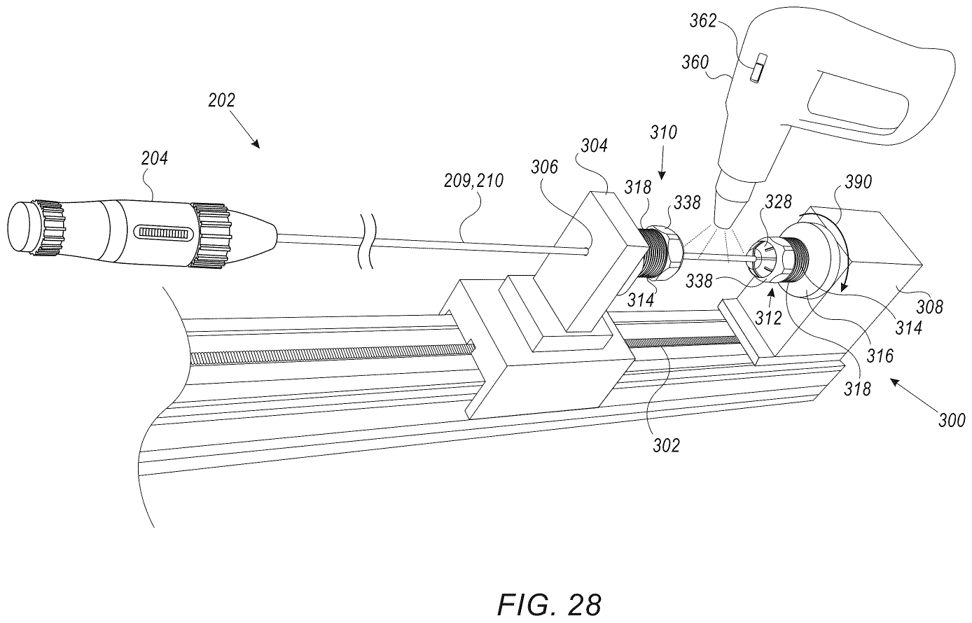

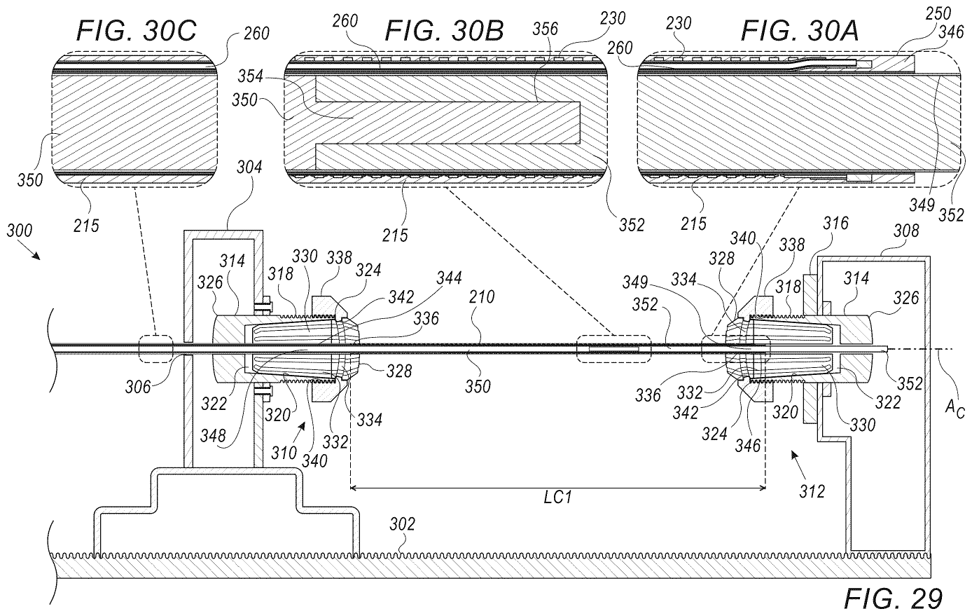

FIGS. 28 and 29 are perspective and sectional side views of an exemplary twisting apparatus 300 that can be used for modifying a steerable delivery catheter 209, as described in further detail herein. FIGS. 30A-30C are enlarged views of selected regions of the sectional side view of FIG. 29. The twisting apparatus 300 can include a stationary clamp 310 and a rotatable clamp 312 axially spaced from each other, wherein the rotatable clamp 312 is configured to rotate relative to the stationary clamp 310. Both the stationary clamp 310 and the rotatable clamp 312 are configured to clamp a steerable delivery catheter 209 extending therethrough, such that the portion of steerable delivery catheter 209 clamped by each of the clamps 310, 312 is immovable relative to the corresponding clamp 310, 312 to which it is attached.

In some examples, the twisting apparatus 300 can include a stationary clamp holder 304 comprising the stationary clamp 310, and a rotational clamp holder 308 comprising the rotatable clamp 312.

As shown in FIG. 28-29, the steerable delivery catheter 209 can be extended through the clamps 310, 312 of the twisting apparatus 300, with each of the clamps 310, 312 clamped over the portion of the steerable delivery catheter 209 passing therethrough. In some examples, the stationary clamp holder 304 can include a pass-through opening 306 aligned with the clamps 310, 312, through which the steerable delivery catheter 209 can be inserted to extend it through the clamps 310, 312, so each of the clamps 310, 312 can be clamped around the portion of the steerable delivery catheter 209 extending therethrough.

The portion of the steerable delivery catheter 209 extending between both clamps 310, 312 includes at least part of the slotted tube 229, and in some examples, the entire length of the slotted tube 229, which can be also referred to, in some examples, as the length LC1 of the distal section 209′ of the steerable delivery catheter 209 (i.e., the section that includes the slotted tube 229), as illustrated in FIG. 29. In some examples, at least one of the stationary clamp holder 304 and/or the rotational clamp holder 308 can be movable towards or away each other over a rack 302 of the twisting apparatus 300, to allow for adjustment of the distance between the clamps 310, 312 when unclamped over the steerable delivery catheter 209, to include a desired length of the steerable delivery catheter 209 therebetween.

In some examples, each of the clamps 310, 312 includes a collet 328 extending, at least partially, through an adapter 314, and a nut 338 engaged with an end of the adapter 314. The collet 328 can include a tapered collet base 330 defining a plurality of axial slots 332, and an annular recess 334. The adapter 314 extends between a first end portion 324 that can be exposed out of the corresponding clamp holder 304 or 308, and a second end portion 326 that can be attached tom or reside in, the corresponding clamp holder 304 or 308. The adapter 314 further include an outer threading 318 extending from its first end portion 326, and a tapered bore 322 defining an inner surface 320, shaped to accommodate a portion of the tapered collet base 330 therein. The nut 338 includes an inner threading 340 engaged with the outer threading 318 of the adapter 314, and an annular flange 342 extending into the annular recess 334 of the collet 328.

The collet 328 defines a collet bore 336 that initially is sized to allow free axial movement of a steerable delivery catheter 209 therethrough. Once the steerable delivery catheter 209 is positioned at a desired axial position, extending through the collet bore 336, the collet 328 can be clamped thereover. Threading the nut 338 around the adaptor 314 causes the tapered collet base 330 to move deeper into the tapered bore 322 of the adapter 314, which causes the elongated fingers or arms defined between the axial slots 332 of the tapered collet bore 330 to move radially inwardly, into engagement with the steerable delivery catheter 209 in a manner that locks the steerable delivery catheter 209 inside the collet 328.

In some examples, the twisting apparatus 300 further comprises an actuator, such as a rotatable knob 316, configured to rotate the rotatable clamp 312 around central longitudinal axis A_{C}. The rotatable knob 316 can be either directly attached to the rotatable clamp 312, or via intermediate components, such as a gear train, configured to transmit rotational movement of the knob 316 to rotation of the clamp 312, for example in circumferential direction 390 indicated in FIG. 28. Since the collet 328 applies a clamping pressure to the steerable delivery catheter 209, the portion of the steerable delivery catheter 209, which can be at, or in close proximity to catheter distal end 228, causes the distal portion of the steerable delivery catheter 209, clamped by the rotatable clamp 312, to move in the same rotation direction therewith.

In contrast to the rotatable clamp 312, the stationary clamp 310 may remain unmoved, forcing the portion of the steerable delivery catheter 209 clamped thereby, which is proximal to the portion of the steerable delivery catheter 209 attached to the rotatable clamp 312, to maintain its angular orientation without being able to move in any direction. Thus, the relative rotational movement of the portion of the steerable delivery catheter 209 attached to the rotatable clamp, for example in circumferential direction 390, relative to the portion attached to the stationary clamp 310, will result in twisting of the portion of the steerable delivery catheter 209 extending between both clamps 310, 312.

In some examples, a mandrel can be inserted through the primary lumen 212 of steerable delivery catheter 209 at the regions clamped by clamps 310, 312. The polymeric layer 214 of the steerable delivery catheter 209 can be relatively soft and/or flexible, such that the clamping pressure applied by a collet 328 can lead to inward collapse of the steerable delivery catheter 209. Thus, insertion of a mandrel into the primary lumen 212 of steerable delivery catheter 209 can support the steerable delivery catheter 209 against the clamping pressure, sandwiching the steerable delivery catheter 209 between the collet 328 and the mandrel.

Utilizing a single mandrel that extends both through the portions of the steerable delivery catheter 209 clamped by the stationary clamp 310 and the portion clamped by the rotational clamp 312 can lead to varying frictional forces developed between the outer surface of the mandrel and the inner surface of the steerable delivery catheter 209. Thus, in some examples, two mandrel are used, a first mandrel 350 extending through a portion of the steerable delivery catheter 209 clamped by the stationary clamp 310, and a second mandrel 352 extending through a portion of the steerable delivery catheter 209 clamped by the rotational clamp 312. The first 350 and second 352 mandrels can be rotatably coupled to each other, and axially aligned with each other, such that the second mandrel 352 can move in a circumferential direction 390 along with the portion of the steerable delivery catheter 209 clamped thereagainst by the rotational clamp 312, while the first mandrel 350 can remain circumferentially immovable.

In some examples, an alignment member 354, which can be a smaller-diameter extension that extends from one of the mandrels, can extend into an alignment bore 356 formed in the other one of the mandrels, configured to axially align both mandrels with each other. In the example illustrated in FIG. 30B, the first mandrel 350 is shown to include the alignment member 354 extending therefrom into an alignment bore 356 of the second mandrel 352. It is to be understood that this configuration is shown by way of illustration and not limitation, and that in some examples, the alignment member 354 can extend from the second mandrel 352 into an alignment bore 356 of the first mandrel 350.

While clamps 310, 312 that include collets 328 are described above and illustrated in FIGS. 28-29, it is to be understood that any of the clamps 310, 312 can include any other type of clamping mechanism configured to clamp around the steerable delivery catheter 209 in a manner that prevent relative movement in any direction between the clamp and the respective portion of the steerable delivery catheter 209 clamped thereby.

In some examples, a fan 360 or other heating and/or colling device can be utilized, either as part of the twisting apparatus 300 or as a separate device that can be used in combination with the twisting apparatus 300. FIG. 28 illustrates an exemplary fan 360 that can be in the form of a heat generator, such as a heat blower, configured to direct hot air to the region of the steerable delivery catheter 209 extending between the clamps 310, 312, so as to elevate the temperature of the polymeric layer 215.

In some examples, the fan 360 can be configured to transition between heating and cooling states thereof, such as by including a switch 362 that allows a user to set the desired state of the fan 360. In such examples, the fan 360 can be switched to a cooling state and used to direct cold air to the region of the steerable delivery catheter 209 extending between the clamps 310, 312, so as to affix the polymeric layer 215 in its twisted state. In some examples, separate devices can be used for heating and cooling the region of the steerable delivery catheter 209 extending between the clamps 310, 312.

In some examples, the modification further comprises forming or changing a helical angle of the pull-wire lumen 218. In some examples, the modification further comprises forming a helical angle of the pull-wire lumen 218. In some examples, the modification further comprises forming or changing a helical angle of the braid 222. In some examples, the modification further comprises forming a helical angle of the braid 222.

An example method of modifying the steerable delivery catheter 209 can include a step of providing a delivery catheter 209. The delivery catheter 209 can include a primary lumen 212 defining a central longitudinal axis A_{C}. The delivery catheter 209 can include polymeric layer 215 disposed around the primary lumen 212. The delivery catheter 209 can include a slotted tube 229. The slotted tube 229 can be embedded in the polymeric layer 215. The slotted tube 229 can be disposed around the primary lumen 212 along at least a distal segment 209′ of the steerable delivery catheter 209. The slotted tube 229 can comprise a plurality of slots 232 axially spaced from each other. Each slot can define a slot circumferential center 238 between circumferential ends of the slots 232. A distal-most slot can be circumferentially aligned with an adjacent slot along the length of the slotted tube 229.

The method of modifying the steerable delivery catheter 209 can include a step of holding the catheter 209 at two holding points, for example, as shown in FIG. 29. The step can include holding the polymeric layer 215 at a first holding point 344. The step can include holding the polymeric layer 215 at a second holding point 346. The first holding point 344 can be axially spaced from the second holding point 346. The second holding point 346 can be in mechanical communication with a distal end portion 230a of the slotted tube 229. The first holding point 344 can be in mechanical communication with a proximal end portion 230c and/or an intermediate portion 230b of the slotted tube. The first holding point 344 can be in mechanical communication with a proximal end portion 230c of the slotted tube 229. The first holding point 344 can be in mechanical communication with an intermediate portion 230b of the slotted tube 229.

The method of modifying the steerable delivery catheter 209 can include a step of imparting energy to a portion of the polymeric layer 215. The portion of the polymeric layer 215, can be in mechanical communication with the slotted tube 229. The step can result in increasing the malleability of the portion of the polymeric layer 215.

The method of modifying the steerable delivery catheter 209 can include a step of rotating the steerable delivery catheter 209. The step can include rotating one of the first 344 and second 346 holding points around the central longitudinal axis A_{C} relative to the other holding point. The rotation can result in twisting the polymeric layer 215 and slotted tube 229 radially around the central longitudinal axis A_{C}. The rotation can result in the circumferential center 238 of the distal-most slot being circumferentially offset from the circumferential center 238 of the adjacent slot by a helix angle.

An example method of modifying the steerable delivery catheter 209 can include:
(a) providing a delivery catheter 209, which comprises:
   a primary lumen 212 defining a central longitudinal axis A_{C};
   a polymeric layer 215 disposed around the primary lumen 212;
   a slotted tube 229 embedded in the polymeric layer 215 and disposed around the primary lumen 212 along at least a distal segment 209′ of the steerable delivery catheter 209, the slotted tube 229 comprising a plurality of slots 232 axially spaced from each other, wherein each slot defines a slot circumferential center 238 between circumferential ends of the slots 232, wherein a distal-most slot is circumferentially aligned with an adjacent slot along the length of the slotted tube 229;
(b) holding the polymeric layer 215 at a first holding point 344 and at a second holding point 346, wherein the first holding point 344 is axially spaced from the second holding point 346, wherein the second holding point 346 is in mechanical communication with a distal end portion 230a of the slotted tube 229 and the first holding point 344 is in mechanical communication with a proximal end portion 230c and/or an intermediate portion 230b of the slotted tube;
(c) imparting energy to a portion of the polymeric layer 215, which is in mechanical communication with the slotted tube 229, thereby increasing the malleability of the portion of the polymeric layer 215;
(d) rotating one of the first 344 and second 346 holding points around the central longitudinal axis A_{C} relative to the other holding point, thereby twisting the polymeric layer 215 and slotted tube 229 radially around the central longitudinal axis A_{C}, so that the circumferential center 238 of the distal-most slot is circumferentially offset from the circumferential center 238 of the adjacent slot by a helix angle.

The alphabetical designation of different method steps herein is provided for the sake of clarity and convenient reference to specific method steps. It is to be understood that the definitions of methods described herein are not limited to the alphabetical order.

Reference is now made to step (a) of the present method. In some examples, the present process comprises step (a) of providing a delivery catheter. In some examples, the present process comprises step (a) of providing a delivery catheter 209.

In some examples, the steerable delivery catheter 209 provided in step (a) includes a slotted tube 229 as described above, having a plurality of slots 232 circumferentially aligned with each other along the length of the slotted tube 229.

In some examples, step (a) comprises providing a delivery catheter 209, which comprises:
a primary lumen 212 defining a central longitudinal axis A_{C};
a polymeric layer 215 disposed around the primary lumen 212;
a slotted tube 229 embedded in the polymeric layer 215 and disposed around the primary lumen 212 along at least a distal segment 209′ of the steerable delivery catheter 209, the slotted tube 229 comprising a plurality of slots 232 axially spaced from each other, wherein each slot defines a slot circumferential center 238 between circumferential ends of the slot 232, wherein a distal-most slot is circumferentially aligned with an adjacent slot along the length of the slotted tube 229.

The term “polymeric layer” as used herein, refers to any layer, which comprises at least one natural or artificial polymer. In some examples, the polymeric layer consists of the natural and/or artificial polymer(s). In some examples, the polymer is artificial. Exemplary polymers constituting the polymeric layer

In some examples, the polymeric layer 215 has a temperature-dependent Ductility. In some examples, at least one of the polymers of the polymeric layer 215 has a temperature-dependent Ductility. In some examples, the polymeric layer 215 has a temperature-dependent malleability. In some examples, at least one of the polymers of the polymeric layer 215 has a temperature-dependent malleability.

The term “ductility” refers to a mechanical property commonly described as a material's amenability to drawing. In materials science, ductility is defined by the degree to which a material can sustain plastic deformation under tensile stress before failure. The term “malleability” refers to a similar mechanical property, which is characterized by a material's ability to deform plastically without failure under compressive stress.

In some examples, the polymeric layer 215 has a temperature-dependent stiffness. In some examples, at least one of the polymers of the polymeric layer 215 has a temperature-dependent stiffness.

The term “stiffness” refers to the extent to which an object resists deformation in response to an applied force. The complementary terms of stiffness are “flexibility” or “pliability”, which are used interchangeably herein. The more flexible or pliable an object is, the less stiff it is.

In some examples, the polymeric layer 215 comprises at least one polymer having a glass transition temperature in the range of 50° C. to 250° C., including each value and sub-range within the specified range. In some examples, the glass transition temperature is at least 50° C. In some examples, the glass transition temperature is at least 55° C. In some examples, the glass transition temperature is at least 60° C. In some examples, the glass transition temperature is no more than 250° C. In some examples, the glass transition temperature is no more than 200° C.

As used herein, the term “glass transition temperature” means the temperature at which a polymer transitions from its rigid state to a flexible or rubber-like state.

In some examples, at least 15% of the slots 232 in step (a) are circumferentially aligned with each other along the length of the slotted tube 229. In some examples, at least 25% of the slots 232 in step (a) are circumferentially aligned with each other along the length of the slotted tube 229. In some examples, at least 35% of the slots 232 in step (a) are circumferentially aligned with each other along the length of the slotted tube 229. In some examples, at least 45% of the slots 232 in step (a) are circumferentially aligned with each other along the length of the slotted tube 229. In some examples, at least 55% of the slots 232 in step (a) are circumferentially aligned with each other along the length of the slotted tube 229. In some examples, at least 65% of the slots 232 in step (a) are circumferentially aligned with each other along the length of the slotted tube 229. In some examples, at least 75% of the slots 232 in step (a) are circumferentially aligned with each other along the length of the slotted tube 229. In some examples, at least 85% of the slots 232 in step (a) are circumferentially aligned with each other along the length of the slotted tube 229. In some examples, at least 95% of the slots 232 in step (a) are circumferentially aligned with each other along the length of the slotted tube 229. In some examples, each one of the slots 232 of the slotted tube 229 of the delivery catheter 209 provided in step (a) are circumferentially aligned with each other along the length of the slotted tube 229.

Reference is now made to step (b) of the present method. In some examples, the present process comprises step (b) of holding a polymeric layer 215 of a delivery catheter 209 at a first holding point 344. In some examples, the present process comprises step (b) of holding a polymeric layer 215 of a delivery catheter 209 at a second holding point 346. In some examples, the present process comprises step (b) of holding the polymeric layer 215 at a first holding point 344 and at a second holding point 346.

In some examples, the first holding point 344 is axially spaced from the second holding point 346. In some examples, the second holding point 346 is in mechanical communication with a distal end portion 230a of a slotted tube 229 of a steerable delivery catheter 209. In some examples, a first holding point 344 of polymeric layer 215 of a delivery catheter 209 is in mechanical communication with a proximal end portion 230c and/or an intermediate portion 230b of a slotted tube of a steerable delivery catheter 209.

In some examples, the present process comprises step (b) of holding the polymeric layer 215 at a first holding point 344 and at a second holding point 346, wherein the first holding point 344 is axially spaced from the second holding point 346, wherein the second holding point 346 is in mechanical communication with a distal end portion 230a of the slotted tube 229 and the first holding point 344 is in mechanical communication with a proximal end portion 230c and/or an intermediate portion 230b of the slotted tube.

FIG. 29 illustrates the holding of the polymeric layer 215, including the first holding point 344 and the second holding point 346, which are shown spaced from each other as defined herein.

Although FIG. 29 shows two holding points, it is contemplated that the polymeric layer 215 can be held in more than two holding points. In some examples, step (b) comprises holding the polymeric layer 215 only at the first holding point 344 and at the second holding point 346.

In some examples, the second holding point 236 is in mechanical communication with a distal end portion 230a of the slotted tube 229. In some examples, the first holding point 344 is in mechanical communication with a proximal end portion 230c and/or an intermediate portion 230b of the slotted tube 229. In some examples, the first holding point 344 is in mechanical communication with a proximal end portion 230c of the slotted tube 229.

the term “mechanical communication” generally refers to components being in direct physical contact with each other or being in indirect physical contact with each other where movement of one component affect the position of the other.

In some examples, FIG. 29 represent an example of the present method, in which the first holding point 344 is located at the portion of the polymeric layer 215, in which the proximal end of the slotted tube 230c is embedded. Also, FIG. 29 represent an example of the present method, in which the second holding point 346 is located at the portion of the polymeric layer 215, in which the distal end of the slotted tube 230a is embedded (see FIG. 29). FIG. 30C, which focuses on a more proximal section of the catheter 209, shows that in this example, the slotted tube 229 terminates approximately at the first holding point 344, and is not embedded in the more proximal sections of the catheter. Thus, FIG. 29 represent an example of the present method, in which the slotted tube 229 is held in step (b) approximately at its two ends. Without wishing to be bound by any theory of mechanism of action, this configuration may enable a twisting of the slotted tube 229 throughout its length during steps (c) and (d).

Thus, in some examples, at least part of a length of the slotted tube 229 is axially disposed between the first holding point 344 and the second holding point 346. In some examples, at least 5% of the length of the slotted tube 229 is axially disposed between the first holding point 344 and the second holding point 346. In some examples, at least 10% of the length of the slotted tube 229 is axially disposed between the first holding point 344 and the second holding point 346. In some examples, at least 15% of the length of the slotted tube 229 is axially disposed between the first holding point 344 and the second holding point 346. In some examples, at least 25% of the length of the slotted tube 229 is axially disposed between the first holding point 344 and the second holding point 346. In some examples, at least 33% of the length of the slotted tube 229 is axially disposed between the first holding point 344 and the second holding point 346. In some examples, at least 40% of the length of the slotted tube 229 is axially disposed between the first holding point 344 and the second holding point 346. In some examples, at least 45% of the length of the slotted tube 229 is axially disposed between the first holding point 344 and the second holding point 346. In some examples, at least 50% of the length of the slotted tube 229 is axially disposed between the first holding point 344 and the second holding point 346. In some examples, at least 67% of the length of the slotted tube 229 is axially disposed between the first holding point 344 and the second holding point 346. In some examples, at least 75% of the length of the slotted tube 229 is axially disposed between the first holding point 344 and the second holding point 346. In some examples, at least 85% of the length of the slotted tube 229 is axially disposed between the first holding point 344 and the second holding point 346. In some examples, at least 95% of the length of the slotted tube 229 is axially disposed between the first holding point 344 and the second holding point 346.

In some examples, the entire length of the slotted tube 229 is axially disposed between the first holding point 344 and the second holding point 346.

In some examples, the distal end portion 230a of the slotted tube is embedded within the polymeric layer 215 proximally to the second holding point 346. This example is shown in FIG. 29 and in in FIG. 30A.

In some examples, an axial distance between the first holding point 344 and the second holding point 346 is at least 100% greater than an axial distance between the second holding point 346 and the distal end portion of the slotted tube 230a. In some examples, an axial distance between the first holding point 344 and the second holding point 346 is at least 250% greater than an axial distance between the second holding point 346 and the distal end portion of the slotted tube 230a. In some examples, an axial distance between the first holding point 344 and the second holding point 346 is at least 500% greater than an axial distance between the second holding point 346 and the distal end portion of the slotted tube 230a. In some examples, an axial distance between the first holding point 344 and the second holding point 346 is at least 1000% greater than an axial distance between the second holding point 346 and the distal end portion of the slotted tube 230a. In some examples, an axial distance between the first holding point 344 and the second holding point 346 is at least 2500% greater than an axial distance between the second holding point 346 and the distal end portion of the slotted tube 230a. In some examples, an axial distance between the first holding point 344 and the second holding point 346 is at least 5000% greater than an axial distance between the second holding point 346 and the distal end portion of the slotted tube 230a. In some examples, an axial distance between the first holding point 344 and the second holding point 346 is at least 10000% greater than an axial distance between the second holding point 346 and the distal end portion of the slotted tube 230a. In some examples, an axial distance between the first holding point 344 and the second holding point 346 is at least 100000% greater than an axial distance between the second holding point 346 and the distal end portion of the slotted tube 230a.

In some examples, LC1 is at least 100% greater than an axial distance between the second holding point 346 and the distal end portion of the slotted tube 230a. In some examples, LC1 is at least 250% greater than an axial distance between the second holding point 346 and the distal end portion of the slotted tube 230a. In some examples, LC1 is at least 500% greater than an axial distance between the second holding point 346 and the distal end portion of the slotted tube 230a. In some examples, LC1 is at least 1000% greater than an axial distance between the second holding point 346 and the distal end portion of the slotted tube 230a. In some examples, LC1 is at least 2500% greater than an axial distance between the second holding point 346 and the distal end portion of the slotted tube 230a. In some examples, LC1 is at least 5000% greater than an axial distance between the second holding point 346 and the distal end portion of the slotted tube 230a. In some examples, LC1 is at least 10000% greater than an axial distance between the second holding point 346 and the distal end portion of the slotted tube 230a. In some examples, LC1 is at least 100000% greater than an axial distance between the second holding point 346 and the distal end portion of the slotted tube 230a.

In some examples, the proximal end portion 230c of the slotted tube is embedded within the polymeric layer 215 proximally to the first holding point 344. This example is shown in FIG. 29.

In some examples, an axial distance between the first holding point 344 and the second holding point 346 is at least 100% greater than an axial distance between the first holding point 344 and the proximal end portion of the slotted tube 230c. In some examples, an axial distance between the first holding point 344 and the second holding point 346 is at least 250% greater than an axial distance between the first holding point 344 and the proximal end portion of the slotted tube 230c. In some examples, an axial distance between the first holding point 344 and the second holding point 346 is at least 500% greater than an axial distance between the first holding point 344 and the proximal end portion of the slotted tube 230c. In some examples, an axial distance between the first holding point 344 and the second holding point 346 is at least 1000% greater than an axial distance between the first holding point 344 and the proximal end portion of the slotted tube 230c. In some examples, an axial distance between the first holding point 344 and the second holding point 346 is at least 5000% greater than an axial distance between the first holding point 344 and the proximal end portion of the slotted tube 230c.

In some examples, LC1 is at least 100% greater than an axial distance between the first holding point 344 and the proximal end portion of the slotted tube 230c. In some examples, LC1 is at least 250% greater than an axial distance between the first holding point 344 and the proximal end portion of the slotted tube 230c. In some examples, LC1 is at least 500% greater than an axial distance between the first holding point 344 and the proximal end portion of the slotted tube 230c. In some examples, LC1 is at least 1000% greater than an axial distance between the first holding point 344 and the proximal end portion of the slotted tube 230c. In some examples, LC1 is at least 5000% greater than an axial distance between the first holding point 344 and the proximal end portion of the slotted tube 230c.

In some examples, an axial distance between the first holding point 344 and the second holding point 346 is at least 10000% greater than an axial distance between the first holding point 344 and the proximal end portion of the slotted tube 230c. In some examples, an axial distance between the first holding point 344 and the second holding point 346 is at least 50000% greater than an axial distance between the first holding point 344 and the proximal end portion of the slotted tube 230c. In some examples, an axial distance between the first holding point 344 and the second holding point 346 is at least 100000% greater than an axial distance between the first holding point 344 and the proximal end portion of the slotted tube 230c.

In some examples, LC1 is at least 10000% greater than an axial distance between the first holding point 344 and the proximal end portion of the slotted tube 230c. In some examples, LC1 is at least 50000% greater than an axial distance between the first holding point 344 and the proximal end portion of the slotted tube 230c. In some examples, LC1 is at least 100000% greater than an axial distance between the first holding point 344 and the proximal end portion of the slotted tube 230c.

In general, in some examples, the catheter 209 is an elongated hollow structure, which has the primary lumen 212 that is defining the central longitudinal axis A_{C}. As shown in the example of FIG. 29, a rod 350, 352 may be inserted into the primary lumen 212 during the method of the present disclosure. Also shown in FIG. 29 is a twisting apparatus 300, which holds the polymeric layer 215 at least during step (b) at the first 344 and second 346 holding points. The twisting apparatus 300 is described herein in more detail. It includes a first clamp 310 and a second clamp 312, which hold the first 344 and second 346 holding points, respectively, in step (b).

However, being a hollow structure with the primary lumen 212 involves a risk of collapsing the catheter 209 inward and subsequent deformation, as a result of the physical pressure applied to the first 344 and second 346 holding points by the first 344 and second 346 clamps.

The rod 350, 352 inserted into the primary lumen 212 during the holding of the polymeric layer 215 of the catheter 209 at the first 344 and second 346 holding points, is used, in some examples, in order to prevent such collapse and related deformation. The collapse of the polymeric layer 215 and entire catheter 209 inwards is prevented by the rod 350, 352, which may be a simple metal mandrel that is sandwiched by the catheter 209.

Thus, in some examples, step (b) further comprises preventing from the delivery catheter 209 to substantially collapse inwards upon the holding at the first holding point 344 and/or to substantially collapse inwards upon the holding at the second holding point 346. Each possibility represents a separate example.

In some examples, step (b) further comprises preventing from the delivery catheter 209 to substantially collapse inwards upon the holding at the first holding point 344. In some examples, step (b) further comprises preventing from the delivery catheter 209 to substantially collapse inwards upon the holding at the second point 346. In some examples, step (b) further comprises preventing from the delivery catheter 209 to substantially collapse inwards upon the holding at the first holding point 344. In some examples, wherein step (b) further comprises preventing from the delivery catheter 209 to substantially collapse inwards upon the holding at the second point 346 and at the first holding point 344.

In some examples, step (b) further comprises maintaining at least one rod 350, 352 within the primary lumen 212. In some examples, the rod 350, 352 is a mandrel.

In some examples, step (b) further comprises inserting at least one rod 350, 352 into the primary lumen 212.

It is to be understood that the timing of the insertion of the rod(s) 350, 352 may be considered as either in step (a) or in step (b) as long as during step (b), in some examples, the rod(s) 350, 352 is maintained within the primary lumen 212. For example, a preliminary procedure of manufacturing the delivery catheter 209 circumferentially around the at least one rod 350, 352, may take place prior to step (a).

Thus, in some examples, step (a) further comprises maintaining at least one rod 350, 352 within the primary lumen 212. In some examples, step (a) further comprises inserting at least one rod 350, 352 into the primary lumen 212. In some examples, the method further comprises maintaining at least one rod 350, 352 within the primary lumen 212. In some examples, the method further comprises inserting at least one rod 350, 352 into the primary lumen 212.

In some examples, the first 344 and second 346 holding points are maintained held during step (c). In some examples, the first 344 and second 346 holding points are maintained held during step (d). In some examples, the first 344 and second 346 holding points are maintained held during step (e).

Therefore, in some examples, the method further comprises maintaining the at least one rod 350, 352 within the primary lumen 212 during steps (b), (c) and (d). In some examples, the method further comprises maintaining the at least one rod 350, 352 within the primary lumen 212 during steps (b), (c), (d) and (e). In some examples, the method further comprises maintaining the at least one rod 350, 352 within the primary lumen 212 during steps (a), (b), (c) and (d). In some examples, the method further comprises maintaining the at least one rod 350, 352 within the primary lumen 212 during steps (a), (b), (c), (d) and (e).

In some examples, step (c) further comprises maintaining at least one rod 350, 352 within the primary lumen 212. In some examples, the method further comprises maintaining the at least one rod 350, 352 within the primary lumen 212 during both steps (b) and (c). In some examples, step (d) further comprises maintaining at least one rod 350, 352 within the primary lumen 212. In some examples, step (e) further comprises maintaining at least one rod 350, 352 within the primary lumen 212.

In some examples, the at least one rod 350, 352 is a metal rod. The term “metal rod” is intended to mean any rod that comprises at least one metal. For example, a mandrel made of either pure iron or an iron alloy (e.g., steal) is considered a metal rod, as used herein.

In some examples, the at least one rod 350, 352 has a melting point higher than the temperature of the polymeric layer 215 during step (c). In some examples, the at least one rod 350, 352 has a melting point at least 50° C. higher than the temperature of the polymeric layer 215 during step (c). The temperature of the polymeric layer in step (c) (e.g., T^{c}) is discussed below when reference is made to step (c). However, in examples, in which the temperature is elevated, the at least one rod 350, 352 should maintain sufficient mechanical integrity as a solid.

In some examples, the at least one rod 350, 352 has a first holding point 348, wherein step (b) comprises maintaining the rod first holding point 348 is laterally substantially in parallel to the polymeric layer first holding point 344.

In some examples, holding the polymeric layer 215 at the first holding point 344 includes applying mechanical pressure on the polymeric layer first holding point 344 radially inward, thereby also applying mechanical pressure on the rod first holding point 348 radially inward.

In some examples, the at least one rod 350, 352 has a second holding point 349, wherein step (b) comprises maintaining the rod second holding point 349 is laterally substantially in parallel to the polymeric layer first holding point 344. In some examples, holding the polymeric layer 215 at the second holding point 346 includes applying mechanical pressure on the polymeric layer second holding point 346 radially inward, thereby also applying mechanical pressure on the rod second holding point 349 radially inward.

The example portrayed by FIG. 29 presents a second rod 352 having a recess 356 and a first rod 350 having a protrusion that is fitting into the recess 356. This, in some examples, enables the twisting of step (d), which is elaborated below. In short, during step (d), in some examples, the second polymeric layer holding point 346 is rotated in unison with the second rod 352, while the first polymeric layer holding point 344 remains stationary, thereby twisting the delivery catheter 209.

In some examples, step (b) comprises maintaining a first rod and a second rod within the primary lumen. In some examples, the first rod 350 is a metal rod. In some examples, the first rod 350 has a melting point higher than T^{c}. In some examples, the first rod 350 has a melting point at least 50° C. higher than T^{c}. In some examples, the second rod 352 is a metal rod. In some examples, the second rod 352 has a melting point higher than T^{c}. In some examples, the second rod 352 has a melting point at least 50° C. higher than T^{c}.

In some examples, the method provides two rods only, the first rod 350 and the second rod 352.

In some examples, step (b) comprises maintaining the first rod 350 and the second rod 352 substantially axially aligned within the primary lumen 212.

In some examples, the first rod 350 has a proximal end portion and a distal end portion. In some examples, the second rod 352 has a proximal end portion and a distal end portion. In some examples, step (b) comprises maintaining the first rod distal end portion adjacent to the second rod proximal end portion.

In some examples, the first rod distal end portion is in the form of a protrusion 354 and the second rod proximal end portion is in the form of a recess 356, or wherein the first rod distal end portion is in the form of a recess 356 and the second rod proximal end portion is in the form of a protrusion 354. In some examples, the first rod distal end portion is in the form of a protrusion 354 and the second rod proximal end portion is in the form of a recess 356. In some examples, the first rod distal end portion is in the form of a recess 356 and the second rod proximal end portion is in the form of a protrusion 354.

In some examples, the protrusion 354 is inserted within the recess 356 to maintain substantial axial alignment between the first rod 350 and the second rod 352. In some examples, the protrusion 354 is inserted within the recess 356 to maintain substantial axial alignment between the first rod 350 and the second rod 352 during step (b). In some examples, the protrusion 354 is inserted within the recess 356 to maintain substantial axial alignment between the first rod 350 and the second rod 352 during steps (b), (c) and (d).

In some examples, each one of the protrusion 354 and the recess is round shaped, thereby enabling relative rotation between the first rod 350 and the second rod 352.

Reference is now made to step (c) of the present method. In some examples, the present process comprises step (c) of imparting energy to a portion of the polymeric layer 215. In some examples, the portion of the polymeric layer 215 is in mechanical communication with the slotted tube 229. In some examples, step imparting of the energy includes increasing the malleability of the portion of the polymeric layer 215.

An example of step (c) is depicted in FIG. 28. In FIG. 28 the delivery catheter 209 is shown held at the first 344 and second 346 holding points of the polymeric layer 215 by a twisting apparatus 300, which is described in further detail herein. Also shown in FIG. 28 is a heating device 360 in the form of a heat generator (e.g., a heat gun, a hair drier, etc.). The heating device 360 is shown in FIG. 28 to blow heat at the portion of the polymeric layer 215, thereby imparting energy thereon and heating it, in some examples.

In some examples, imparting energy to the portion of the polymeric layer in step (c) includes elevating the temperature of the portion of the polymeric layer. In some examples, the elevated temperature of step (c) is referred herein as temperature TC.

In some examples, step (c) comprises maintaining the portion of the polymeric layer 215 at the elevated temperature for a period of time. In some examples, the period of time is throughout step (c). In some examples, the period of time is throughout steps (c) and (d).

In some examples, step (c) comprises maintaining the portion of the polymeric layer 215 at the elevated temperature for at least 10 seconds. In some examples, step (c) comprises maintaining the portion of the polymeric layer 215 at the elevated temperature for at least 20 seconds. In some examples, step (c) comprises maintaining the portion of the polymeric layer 215 at the elevated temperature for at least 30 seconds. In some examples, step (c) comprises maintaining the portion of the polymeric layer 215 at the elevated temperature for at least 45 seconds. In some examples, step (c) comprises maintaining the portion of the polymeric layer 215 at the elevated temperature for at least 60 seconds. In some examples, step (c) comprises maintaining the portion of the polymeric layer 215 at the elevated temperature for at least 90 seconds. In some examples, step (c) comprises maintaining the portion of the polymeric layer 215 at the elevated temperature for at least 120 seconds. In some examples, step (c) comprises maintaining the portion of the polymeric layer 215 at the elevated temperature for at least 250 seconds. In some examples, step (c) comprises maintaining the portion of the polymeric layer 215 at the elevated temperature for at least 500 seconds. In some examples, maintaining a material at an elevated temperature can facilitate achieving homogeneous temperature and mechanical properties along the length of the heated item.

In some examples, elevating the temperature of the portion of the polymeric layer 215 includes applying external heat to the portion of the polymeric layer 215 using a heating device 360.

As detailed herein, in some examples, imparting energy to the portion of the polymeric layer 215 is involves conventional heating methods. However, in some examples, it is contemplated that the energy is imparted using other methods, such as, but not limited to, irradiation.

In some examples, imparting energy to the portion of the polymeric layer 215 in step (c) includes irradiating the portion of the polymeric layer 215. In some examples, elevating the temperature of the portion of the polymeric layer 215 includes irradiating the portion of the polymeric layer 215.

In some examples, imparting energy to the portion of the polymeric layer 215 in step (c) includes adjusting the temperature of the portion of the polymeric layer 215 to a temperature T^{c}. In some examples, imparting energy to the portion of the polymeric layer 215 in step (c) includes elevating the temperature of the portion of the polymeric layer 215 to a temperature

As defined above, the glass transition temperature, Tg of a polymer is the temperature in which the polymer become flexible or rubber-like state, and therefore, generally more malleable and ductile. In some examples, the purpose of elevating the temperature of the portion of the polymeric layer 215 in step (c) is to make it more malleable and ductile so it can be twisted during step (d) of the present method. Thus, in some examples, it is preferable that the temperature T^{c} is above the glass transition temperature of the polymeric layer 215, of the portion of the polymeric layer 215, and/or any one or more of the polymeric constituents thereof. Another phase transition temperature of the polymeric layer 215, the portion of the polymeric layer 215, and/or any one or more of the polymeric constituents thereof is the melting point of such polymers. In order to maintain the integrity of the delivery catheter 209 as a whole, it is preferable, in some examples, that the polymeric layer 215 will not be heated above its melting point, which may cause its deformation.

In some examples, temperature T^{c} is equal or above the glass transition temperature of at least one polymer comprised within the polymeric layer 215. In some examples, temperature T^{c} is equal or above the glass transition temperature of each polymer comprised within the polymeric layer 215. In some examples, temperature T^{c} is equal or above the glass transition temperature of the polymeric layer 215. In some examples, temperature T^{c} is above the glass transition temperature of at least one polymer comprised within the polymeric layer 215. In some examples, temperature T^{c} is above the glass transition temperature of each polymer comprised within the polymeric layer 215. In some examples, temperature T^{c} is above the glass transition temperature of the polymeric layer 215.

In some examples, temperature T^{c} is equal or above the glass transition temperature of at least one polymer comprised within the portion of the polymeric layer 215. In some examples, temperature T^{c} is equal or above the glass transition temperature of each polymer comprised within the portion of the polymeric layer 215. In some examples, temperature T^{c} is equal or above the glass transition temperature of the portion of the polymeric layer 215. In some examples, temperature T^{c} is above the glass transition temperature of at least one polymer comprised within the portion of the polymeric layer 215. In some examples, temperature T^{c} is above the glass transition temperature of each polymer comprised within the portion of the polymeric layer 215. In some examples, temperature T^{c} is above the glass transition temperature of the portion of the polymeric layer 215.

In some examples, temperature T^{c} is at least 10° C. above the glass transition temperature. In some examples, temperature T^{c} is at least 20° C. above the glass transition temperature. In some examples, temperature T^{c} is at least 30° C. above the glass transition temperature. In some examples, temperature T^{c} is at least 40° C. above the glass transition temperature. In some examples, temperature T^{c} is at least 50° C. above the glass transition temperature. In some examples, temperature T^{c} is at least 60° C. above the glass transition temperature. In some examples, temperature T^{c} is at least 70° C. above the glass transition temperature.

In some examples, temperature T^{c} is at least 80° C. above the glass transition temperature.

In some examples, temperature T^{c} is equal or below the melting point of at least one polymer comprised within the polymeric layer 215. In some examples, temperature T^{c} is equal or below the melting point of each polymer comprised within the polymeric layer 215. In some examples, temperature T^{c} is equal or below the melting point of the polymeric layer 215. In some examples, temperature T^{c} is below the melting point of at least one polymer comprised within the polymeric layer 215. In some examples, temperature T^{c} is below the melting point of each polymer comprised within the polymeric layer 215. In some examples, temperature T^{c} is below the melting point of the polymeric layer 215.

In some examples, temperature T^{c} is equal or below the melting point of at least one polymer comprised within the portion of the polymeric layer 215. In some examples, temperature T^{c} is equal or below the melting point of each polymer comprised within the portion of the polymeric layer 215. In some examples, temperature T^{c} is equal or below the melting point of the portion of the polymeric layer 215. In some examples, temperature T^{c} is below the melting point of at least one polymer comprised within the portion of the polymeric layer 215. In some examples, temperature T^{c} is below the melting point of each polymer comprised within the portion of the polymeric layer 215. In some examples, temperature T^{c} is below the melting point of the portion of the polymeric layer 215.

In some examples, temperature T^{c} is at least 10° C. below the melting point. In some examples, temperature T^{c} is at least 20° C. below the melting point. In some examples, temperature T^{c} is at least 30° C. below the melting point. In some examples, temperature T^{c} is at least 40° C. below the melting point. In some examples, temperature T^{c} is at least 50° C. below the melting point.

In some examples, the portion of the polymeric layer 215 to which energy is imparted to in step (c) is located between the first holding point 344 and the second holding point 346 of the polymeric layer 215. This can be appreciated from FIG. 28.

In some examples, upon the imparting of the energy to the portion of the polymeric layer 215, temperature T^{c} of the portion of the polymeric layer 215 is higher than the temperature of the first holding point 344. In some examples, upon the imparting of the energy to the portion of the polymeric layer 215, temperature T^{c} of the portion of the polymeric layer 215 is higher than the temperature of the second holding point 346. In some examples, upon the imparting of the energy to the portion of the polymeric layer 215, temperature T^{c} of the portion of the polymeric layer 215 is higher than the temperature of the first holding point 344, and higher than the temperature of the second holding point 346.

In some examples, upon the imparting of the energy to the portion of the polymeric layer 215 in step (c) the portion of the polymeric layer 215 becomes at least partially malleable. In some examples, upon the imparting of the energy to the portion of the polymeric layer 215 in step (c) the portion of the polymeric layer 215 becomes malleable. In some examples, upon the imparting of the energy to the portion of the polymeric layer 215 in step (c) the portion of the polymeric layer 215 becomes at least partially ductile. In some examples, upon the imparting of the energy to the portion of the polymeric layer 215 in step (c) the portion of the polymeric layer 215 becomes ductile. In some examples, upon the imparting of the energy to the portion of the polymeric layer 215 in step (c) the portion of the polymeric layer 215 becomes at least partially flexible. In some examples, upon the imparting of the energy to the portion of the polymeric layer 215 in step (c) the portion of the polymeric layer 215 becomes flexible.

In some examples, imparting of the energy to the portion of the polymeric layer 215 includes increasing the malleability of the portion of the polymeric layer 215. In some examples, imparting of the energy to the portion of the polymeric layer 215 includes increasing the ductility of the portion of the polymeric layer 215. In some examples, imparting of the energy to the portion of the polymeric layer 215 includes increasing the flexibility of the portion of the polymeric layer 215.

A quantity commonly used to define ductility in a tension test is the relative elongation (in percent, sometimes denoted as % EL, which is shown below in Equation 1.

$$\begin{matrix}Relative elongation & \\ \% EL = \frac{final gauge length - initial gauge length}{initial gauge length} = \frac{{l}_{f} - {l}_{0}}{{l}_{f}} \times 100. & Equation 1\end{matrix}$$

Thus, in some examples, imparting of the energy to the portion of the polymeric layer 215 includes increasing the relative elongation of the portion of the polymeric layer 215. In some examples, imparting of the energy to the portion of the polymeric layer 215 includes increasing the relative elongation of the portion of the polymeric layer 215 by at least 10%. In some examples, imparting of the energy to the portion of the polymeric layer 215 includes increasing the relative elongation of the portion of the polymeric layer 215 by at least 20%. In some examples, imparting of the energy to the portion of the polymeric layer 215 includes increasing the relative elongation of the portion of the polymeric layer 215 by at least 30%. In some examples, imparting of the energy to the portion of the polymeric layer 215 includes increasing the relative elongation of the portion of the polymeric layer 215 by at least 40%. In some examples, imparting of the energy to the portion of the polymeric layer 215 includes increasing the relative elongation of the portion of the polymeric layer 215 by at least 50%. In some examples, imparting of the energy to the portion of the polymeric layer 215 includes increasing the relative elongation of the portion of the polymeric layer 215 by at least 60%.

As specified herein, the heating in step (c) is intended to increase the malleability, ductility and/or flexibility of the portion of the polymeric layer 215 so that it may be twisted during step (d), in some examples. Thus, in some examples, the elevated temperature (e.g., T^{c}, or a temperature within a close range thereto) may be maintained during step (d). For this purpose, either (I) the heating process continues into step (d) at least for some of the duration of step (d); or (II) during step (d) heating is not performed, but the temperature remains high enough to maintain sufficient malleability, to twist the portion of the polymeric layer 215. Each possibility represents a separate example of the disclosure. The option (I) may be considered as performing steps (c) and (d) at least partially simultaneously.

In some examples, step (c) and step (d) are performed at least partially simultaneously. In some examples, step (c) comprises adjusting the temperature of the portion of the polymeric layer 215 to a temperature T^{c}, which is elevated compared to the ambient temperature, and maintaining the portion of the polymeric layer 215 at elevated temperature throughout step (d). In some examples, wherein the elevated temperature is at least T^{c}−50° C. In some examples, wherein the elevated temperature is at least T^{c}−40° C. In some examples, wherein the elevated temperature is at least T^{c}−30° C. In some examples, wherein the elevated temperature is at least T^{c}−20° C. In some examples, wherein the elevated temperature is at least T^{c}−10° C. In some examples, wherein the elevated temperature is at least T^{c}−5° C. In some examples, the elevated temperature is about To.

Reference is now made to step (d) of the present method. In some examples, the present process comprises step (d) of rotating one of the first 344 and second 346 holding points around the central longitudinal axis A_{C} relative to the other holding point. In some examples, step (d) includes twisting the polymeric layer 215 radially around the central longitudinal axis A_{C}. In some examples, step (d) includes twisting the slotted tube 229 radially around the central longitudinal axis A_{C}. In some examples, step (d) includes twisting the slotted tube 229 radially around the central longitudinal axis A_{C}, so that the circumferential center 238 of the distal-most slot is circumferentially offset from the circumferential center 238 of the adjacent slot by a helix angle.

In some examples, rotating one of the first 344 and second holding points 346 around the central longitudinal axis A_{C} relative to the other holding point in step (d) includes rotating one of the first 344 and second 346 holding points around the central longitudinal axis A_{C} and holding the other holding point static.

In some examples, in FIGS. 28 and 29 an example is shown, which is directed to holding the first holding point 344 static and rotating the second holding point 346.

Thus, in some examples, step (d) comprises rotating the second 346 holding point around the central longitudinal axis A_{C}. In some examples, step (d) comprises holding the first holding point 344 static. In some examples, step (d) comprises rotating the second holding point 346 around the central longitudinal axis A_{C} and holding the first holding point 344 static.

However, various modifications of the example depicted in FIGS. 28 and 29 are contemplated, as follows. In some examples, step (d) comprises rotating the first holding point 344 around the central longitudinal axis A_{C} and holding the second holding point 346 static. In some examples, step (d) comprises rotating both the second holding point 346 and first holding point 344 around the central longitudinal axis A_{C} at different rotation rates. In some examples, step (d) comprises rotating both the second holding point 346 and first holding point 344 around the central longitudinal axis A_{C} at different rotation directions (i.e., one clockwise and the other counter clockwise, when facing the same longitudinal direction).

It is an advantage of the present method, that in relatively simple steps, it enables transition from the traditional non-helical catheter configuration (e.g., as described when referring to FIGS. 8A-F) to the helical configuration catheter (e.g., as described when referring to FIGS. 10A-F and FIGS. 11A-B), wherein the internal components of the delivery catheter (e.g., the pull-wire lumen portion, the polymeric layer 215 and the slotted tube 229) are helically aligned. The helical alignment is important for the 3D maneuvering of the delivery catheter 210 when employed in the body, and is a result of the synchronic twisting of various components of the delivery catheter 210 in step (d), in some examples.

Thus, in some examples, step (d) comprises synchronically twisting the polymeric layer 215 and slotted tube 229 radially.

In some examples, the pull-wire lumen 218 of the delivery catheter 209 provided in step (a) has a portion extending axially at least between the first holding point 344 and the second holding point 346 of the polymeric layer 215. In some examples, step (d) comprises twisting the pull-wire lumen portion radially around the central longitudinal axis A_{C}.

In some examples, step (d) comprises synchronically twisting the pull-wire lumen portion and the polymeric layer 215 radially around the central longitudinal axis. In some examples, step (d) comprises synchronically twisting the pull-wire lumen portion and the slotted tube 229 radially around the central longitudinal axis A_{C}. In some examples, step (d) comprises synchronically twisting the pull-wire lumen portion, the polymeric layer 215 and the slotted tube 229 radially around the central longitudinal axis A_{C}.

In some examples, the braid 222 of the delivery catheter 209 provided in step (a) has a portion extending axially at least between the first holding point 344 and the second holding point 346 of the polymeric layer 215.

In some examples, step (d) comprises twisting the braid portion radially around the central longitudinal axis A_{C}. In some examples, step (d) comprises synchronically twisting the braid portion and the polymeric layer portion radially around the central longitudinal axis A_{C}. In some examples, step (d) comprises synchronically twisting the braid portion and the slotted tube 229 radially around the central longitudinal axis A_{C}. In some examples, step (d) comprises synchronically twisting the braid portion, the polymeric layer portion and the slotted tube 229 radially around the central longitudinal axis A_{C}. In some examples, step (d) comprises synchronically twisting the braid portion, the pull-wire lumen portion, the polymeric layer portion and the slotted tube 229 radially around the central longitudinal axis A_{C}.

In some examples, upon the rotation of step (d) the pull-wire 260 is aligned with the slot circumferential centers 238. In some examples, the rotation of step (d) the pull-wire lumen 218 is aligned with the slot circumferential centers 238.

As shown in FIGS. 28 and 29, the twisting of step (d) may be performed using a dedicated twisting apparatus 300. The twisting apparatus 300 is described herein in further detail.

In some examples, step (b) comprises providing a twisting apparatus 300 and holding the polymeric layer 215 at a first holding point 344 and at a second holding point 346; and step (d) comprises rotating one of the first 344 and second 346 holding points around the central longitudinal axis A_{C} relative to the other holding point using the twisting apparatus 300. In some examples, the holding is performed using the clamps 310, 312 of the twisting apparatus 300.

In some examples, the helix angle formed in step (d) is not greater than 5°.

In some examples, the helix angle formed in step (d) comprises a distal helix angle by which the distal slots 232a are angularly offset from each other. In some examples, the helix angle formed in step (d) comprises an intermediate helix angle by which the intermediate slots 232b are angularly offset from each other. In some examples, the helix angle formed in step (d) comprises a proximal helix angle by which the distal slots 232a are angularly offset from each other.

In some examples, the helix angle formed in step (d) comprises:
a distal helix angle by which the distal slots 232a are angularly offset from each other;
an intermediate helix angle by which the intermediate slots 232b are angularly offset from each other; and
a proximal helix angle by which the distal slots 232a are angularly offset from each other.

In some examples, the distal helix angle is different from at least one of the intermediate helix angle and/or the proximal helix angle. In some examples, a distal-most slot 232b of the intermediate slots 232b is angularly offset from a proximal-most slot 232a of the distal slots 232a by an angle between the distal helix angle and the intermediate helix angle, inclusive. In some examples, distal-most slot of the proximal slots 232c is angularly offset from a proximal-most slot of the intermediate slots 232b by an angle between the intermediate helix angle and the proximal helix angle, inclusive. In some examples, the slots 232 upon the rotation of step (d) are orthogonal to an angled axis A_{A}, defined as an axis which is angled at the helix angle relative to the central longitudinal axis A_{C}.

Reference is now made to step (e) of the present method. In some examples, the present process further comprises step (e) of lowering the temperature of the portion of the polymeric layer 215. It is to be understood that in the context of the present method, the phrase “lowering the temperature of the portion of the polymeric layer” refers to the temperature relative to the temperature of the previous step (c) and/or (d). The temperature may be higher, lower or similar to that of step (b) or (a).

In some examples, lowering the temperature of the portion of the polymeric layer 215 in step (e) includes adjusting the temperature of the portion of the polymeric layer 215 to a temperature T^{e}.

In some examples, temperature T^{e} is equal or below the glass transition temperature of at least one polymer comprised within the polymeric layer 215. In some examples, temperature T^{e} is equal or below the glass transition temperature of each polymer comprised within the polymeric layer 215. In some examples, temperature T^{e} is equal or below the glass transition temperature of the polymeric layer 215. In some examples, temperature T^{e} is below the glass transition temperature of at least one polymer comprised within the polymeric layer 215. In some examples, temperature T^{e} is below the glass transition temperature of each polymer comprised within the polymeric layer 215. In some examples, temperature T^{e} is below the glass transition temperature of the polymeric layer 215.

In some examples, temperature T^{e} is equal or below the glass transition temperature of at least one polymer comprised within the portion of the polymeric layer 215. In some examples, temperature T^{e} is equal or below the glass transition temperature of each polymer comprised within the portion of the polymeric layer 215. In some examples, temperature T^{e} is equal or below the glass transition temperature of the portion of the polymeric layer 215. In some examples, temperature T^{e} is below the glass transition temperature of at least one polymer comprised within the portion of the polymeric layer 215. In some examples, temperature T^{e} is below the glass transition temperature of each polymer comprised within the portion of the polymeric layer 215. In some examples, temperature T^{e} is below the glass transition temperature of the portion of the polymeric layer 215.

In some examples, temperature T^{e} is at least 10° C. below the glass transition temperature. In some examples, temperature T^{e} is at least 20° C. below the glass transition temperature. In some examples, temperature T^{e} is at least 30° C. below the glass transition temperature. In some examples, temperature T^{e} is at least 40° C. below the glass transition temperature. In some examples, temperature T^{e} is at least 50° C. below the glass transition temperature. In some examples, temperature T^{e} is at least 60° C. below the glass transition temperature. In some examples, temperature T^{e} is at least 70° C. below the glass transition temperature.

In some examples, step (e) is intended to cool the portion of the polymeric layer 215 so that it is lee malleable and maintains its twisted configuration, in some examples.

In some examples, temperature T^{e} is no more than 35° C. In some examples, temperature T^{e} is about ambient temperature. In some examples, lowering the temperature in step (e) comprises maintaining the portion of the polymeric layer 215 at ambient temperature until it is cooled. In some examples, the ambient temperature is about 25° C.

In some examples, the portion of the polymeric layer 215 of which the temperature is lowered in step (e) is located between the first holding point 344 and the second holding point 346 of the polymeric layer 215.

In some examples, upon the lowering of the temperature of the portion of the polymeric layer 215, a temperature T^{e} of the portion of the polymeric layer 215 is similar to the temperature of the first holding point 344. In some examples, upon the lowering of the temperature of the portion of the polymeric layer 215, a temperature T^{e} of the portion of the polymeric layer 215 is similar to the temperature of the second holding point 346. In some examples, upon the lowering of the temperature of the portion of the polymeric layer 215, a temperature T^{e} of the portion of the polymeric layer 215 is similar to the temperature of the first holding point 344 and the temperature of the second holding point 346.

In some examples, lowering the temperature of the portion of the polymeric layer 215 in step (e) includes reducing the malleability of the portion of the polymeric layer 215. In some examples, lowering the temperature of the portion of the polymeric layer 215 in step (e) includes reducing the ductility of the portion of the polymeric layer 215. In some examples, lowering the temperature of the portion of the polymeric layer 215 in step (e) includes reducing the flexibility of the portion of the polymeric layer 215.

In some examples, lowering the temperature of the portion of the polymeric layer 215 in step (e) includes reducing the relative elongation of the portion of the polymeric layer 215. In some examples, lowering the temperature of the portion of the polymeric layer 215 in step (e) includes reducing the relative elongation of the portion of the polymeric layer 215 by at least 5%. In some examples, lowering the temperature of the portion of the polymeric layer 215 in step (e) includes reducing the relative elongation of the portion of the polymeric layer 215 by at least 10%. In some examples, lowering the temperature of the portion of the polymeric layer 215 in step (e) includes reducing the relative elongation of the portion of the polymeric layer 215 by at least 15%. In some examples, lowering the temperature of the portion of the polymeric layer 215 in step (e) includes reducing the relative elongation of the portion of the polymeric layer 215 by at least 20%. In some examples, lowering the temperature of the portion of the polymeric layer 215 in step (e) includes reducing the relative elongation of the portion of the polymeric layer 215 by at least 25%. In some examples, lowering the temperature of the portion of the polymeric layer 215 in step (e) includes reducing the relative elongation of the portion of the polymeric layer 215 by at least 30%.

In some examples, step (e) comprises maintaining the portion of the polymeric layer 215 at ambient temperature for a time period sufficient to significantly reduce the relative elongation of the portion of the polymeric layer 215.

According to Shigley's Mechanical Engineering Design [Budynas, Richard G. (2015). Shigley's Mechanical Engineering Design—10th ed. McGraw Hill. p. 233. ISBN 978-0-07-339820-4] significant denotes about 5% elongation.

In some examples, step (e) comprises immersing the polymeric layer 215 in water at a temperature of no more than 40° C. In some examples, step (e) comprises immersing the polymeric layer 215 in water at a temperature of no more than 35° C. In some examples, step (e) comprises immersing the polymeric layer 215 in water at a temperature of no more than 30° C. In some examples, step (e) comprises immersing the polymeric layer 215 in water at a temperature of no more than 25° C. In some examples, step (e) comprises immersing the polymeric layer 215 in water at a temperature of no more than T^{e}.

In some examples, step (e) comprises immersing the polymeric layer 215 in water and then allowing it to reach ambient temperature in the air.

As detailed herein, the product of the present method is the delivery catheter of the present disclosure, e.g., as described in FIGS. 10A-F and in FIGS. 11A-B.

Some examples relating to the product are described below. It is to be understood that the term “product delivery catheter” refers either to the catheter 210 upon completion of step (d) or to the catheter 210 upon completion of step (e). Thus, the term “product delivery catheter” may be replaced to the catheter 210 upon completion of step (d), in some examples. The term “product delivery catheter” may be replaced to the catheter 210 upon completion of step (d), in some examples.

In some examples, in the product catheter 210, each slot 232 of the slotted tube 229 spans more than 180° of the circumference of the slotted tube 230. In some examples, in the product catheter 210, each slot 232 of the slotted tube 230) spans more than 220° of the circumference of the slotted tube 230. In some examples, in the product catheter 210, each slot 232 of the slotted tube 230 spans more than 270° of the circumference of the slotted tube 230.

In some examples, the product catheter 210 further comprises a backbone 242 opposite to the slot circumferential centers 238, defined by uncut portions of the slotted tube 230 between the circumferential ends of the slots 232. In some examples, the pull-wire 260 extends opposite to the backbone 242. In some examples, the pull-wire lumen 218 extends opposite to the backbone 242. In some examples, the slotted tube 230 further comprises a plurality of opposite cuts 244 axially spaced from each other and extending through the backbone 242. In some examples, the opposite cuts 244 are axially disposed between the slots 232. In some examples, each opposite cut comprises an opening and two slits circumferentially extending therefrom. In some examples, the openings of the opposite cuts are positioned circumferentially opposite to the slot circumferential centers 238. In some examples, the pull-wire 260 extends opposite to the openings of the opposite cuts. In some examples, the pull-wire lumen 218 extends opposite to the openings of the opposite cuts.

In some examples, in the product catheter 210, the slots 232 of the slotted tube 230 comprise distal slots 232a along a distal portion of the slotted tube 230a, and intermediate slots 232b along an intermediate portion of the slotted tube 230b. In some examples, the slots 232 further comprise proximal slots 232c along a proximal portion of the slotted tube 230c. In some examples, the shape of the distal slots 232a is different from the shape of the intermediate slots 232b and/or the proximal slots 232c. In some examples, in the product catheter 210, a slot width of each slot of the distal slots 232a and the intermediate slots 232b increases towards the corresponding slot circumferential center 238. In some examples, the slot width of each slot of the proximal slots 232c is uniform between the corresponding circumferential ends. In some examples, the shape of the distal slots 232a is different from the shape of at least one of the intermediate slots 232b or the proximal slots 232c. In some examples, each slot 232 defines a slot length between its circumferential ends, and wherein the slot length of the distal slots 232a is different from the slot length of the intermediate slots 232b and/or the proximal slots 232c. In some examples, the width of the ribs 240 disposed between the distal slots 232a is different from the width of the ribs 240 disposed between the intermediate slots 232b and/or the proximal slots 232c. In some examples, the ribs 240 between the distal slots 232c are narrower than the ribs 240 between the intermediate slots 232b. In some examples, the ribs 240 between the intermediate slots 232b are narrower than the ribs 240 between the proximal slots 232c.

In some examples, in the product catheter 210, a slot width of at least some of the slots 232 of the slotted tube 230 gradually changes along a length of the slotted tube 230. In some examples, a slot width of at least some subsequent slots 232 of the plurality of slots gradually increases in the distal direction.

### Some Examples of the Disclosed Technology

Some examples of above-described technology are enumerated below. It should be noted that one feature of an example in isolation or more than one feature of the example taken in combination and, optionally, in combination with one or more features of one or more examples below are examples also falling within the disclosure of this application.

Example 1. A delivery assembly comprising:
a delivery apparatus comprising:
   a steerable delivery catheter comprising:
      a primary lumen defining a central longitudinal axis;
      a slotted tube disposed around the primary lumen along at least a distal segment of the steerable delivery catheter, the slotted tube comprising:
         a plurality of slots axially spaced from each other, wherein each slot has a slot width and defines a slot circumferential center between circumferential ends of the slot; and
         a plurality of ribs defined between the slots;
      a pull-wire lumen extending along at least a portion of the steerable delivery catheter; and
      a pull-wire slidingly extending through the pull-wire lumen and attached to the slotted tube;
wherein the circumferential center of each slot is circumferentially offset from the circumferential center of an adjacent slot by a helix angle.

Example 2. The delivery assembly of any example herein, particularly example 1, wherein each slot spans more than 180° of the circumference of the slotted tube.

Example 3. The delivery assembly of any example herein, particularly example 1, wherein each slot spans more than 220° of the circumference of the slotted tube.

Example 4. The delivery assembly of any example herein, particularly example 1, wherein each slot spans more than 270° of the circumference of the slotted tube.

Example 5. The delivery assembly of any example herein, particularly any one of examples 1 to 4, wherein the helix angle is not greater than 15°.

Example 6. The delivery assembly of any example herein, particularly any one of examples 1 to 4, wherein the helix angle is not greater than 10°.

Example 7. The delivery assembly of any example herein, particularly any one of examples 1 to 4, wherein the helix angle is not greater than 5°.

Example 8. The delivery assembly of any example herein, particularly any one of examples 1 to 7, wherein the pull-wire is aligned with the slot circumferential centers.

Example 9. The delivery assembly of any example herein, particularly any one of examples 1 to 8, wherein the pull-wire lumen is aligned with the slot circumferential centers.

Example 10. The delivery assembly of any example herein, particularly any one of examples 1 to 9, wherein the slotted tube further comprises a backbone opposite to the slot circumferential centers, defined by uncut portions of the slotted tube between the circumferential ends of the slots.

Example 11. The delivery assembly of any example herein, particularly example 10, wherein the pull-wire extends opposite to the backbone.

Example 12. The delivery assembly of any example herein, particularly example 10 or 11, wherein the pull-wire lumen extends opposite to the backbone.

Example 13. The delivery assembly of any example herein, particularly any one of examples 10 to 12, wherein the slotted tube further comprises a plurality of opposite cuts axially spaced from each other and extending through the backbone.

Example 14. The delivery assembly of any example herein, particularly example 13, wherein the opposite cuts are axially disposed between the slots.

Example 15. The delivery assembly of any example herein, particularly example 13 or 14, wherein each opposite cut comprises an opening and two slits circumferentially extending therefrom.

Example 16. The delivery assembly of any example herein, particularly example 15, wherein the openings of the opposite cuts are positioned circumferentially opposite to the slot circumferential centers.

Example 17. The delivery assembly of any example herein, particularly example 16, wherein the pull-wire extends opposite to the openings of the opposite cuts.

Example 18. The delivery assembly of any example herein, particularly example 16 or 17, wherein the pull-wire lumen extends opposite to the openings of the opposite cuts.

Example 19. The delivery assembly of any example herein, particularly any one of examples 1 to 18, wherein the slot width of at least one of the plurality of slots increases in size towards the slot circumferential center.

Example 20. The delivery assembly of any example herein, particularly any one of examples 1 to 18, wherein the slot width of at least one of the plurality of slots is uniform between the circumferential ends of the slot.

Example 21. The delivery assembly of any example herein, particularly any one of examples 1 to 20, wherein the slots comprise distal slots along a distal portion of the slotted tube, and intermediate slots along an intermediate portion of the slotted tube.

Example 22. The delivery assembly of any example herein, particularly example 21, wherein the slots further comprise proximal slots along a proximal portion of the slotted tube.

Example 23. The delivery assembly of any example herein, particularly example 22, wherein the shape of the distal slots is different from the shape of the intermediate slots and/or the proximal slots.

Example 24. The delivery assembly of any example herein, particularly example 23, wherein the slot width of each slot of the distal slots and the intermediate slots increases towards the corresponding slot circumferential center.

Example 25. The delivery assembly of any example herein, particularly example 23 or 24, wherein the slot width of each slot of the proximal slots is uniform between the corresponding circumferential ends.

Example 26. The delivery assembly of any example herein, particularly example 22, wherein the shape of the distal slots is different from the shape of at least one of the intermediate slots or the proximal slots.

Example 27. The delivery assembly of any example herein, particularly any one of examples 22 to 26, wherein each slot defines a slot length between its circumferential ends, and wherein the slot length of the distal slots is different from the slot length of the intermediate slots and/or the proximal slots.

Example 28. The delivery assembly of any example herein, particularly any one of examples 22 to 27, wherein the width of the ribs disposed between the distal slots is different from the width of the ribs disposed between the intermediate slots and/or the proximal slots.

Example 29. The delivery assembly of any example herein, particularly example 28, wherein the ribs between the distal slots are narrower than the ribs between the intermediate slots.

Example 30. The delivery assembly of any example herein, particularly example 28 or 29, wherein the ribs between the intermediate slots are narrower than the ribs between the proximal slots.

Example 31. The delivery assembly of any example herein, particularly any one of examples 22 to 30, wherein the helix angle comprises:
a distal helix angle by which the distal slots are angularly offset from each other;
an intermediate helix angle by which the intermediate slots are angularly offset from each other; and
a proximal helix angle by which the distal slots are angularly offset from each other.

Example 32. The delivery assembly of any example herein, particularly example 31, wherein the distal helix angle is different from at least one of the intermediate helix angle and/or the proximal helix angle.

Example 33. The delivery assembly of any example herein, particularly example 31 or 32, wherein a distal-most slot of the intermediate slots is angularly offset from a proximal-most slot of the distal slots by an angle between the distal helix angle and the intermediate helix angle, inclusive.

Example 34. The delivery assembly of any example herein, particularly any one of examples 31 to 33, wherein a distal-most slot of the proximal slots is angularly offset from a proximal-most slot of the intermediate slots by an angle between the intermediate helix angle and the proximal helix angle, inclusive.

Example 35. The delivery assembly of any example herein, particularly any one of examples 1 to 34, wherein the slots are orthogonal to an angled axis, defined as an axis which is angled at the helix angle relative to the central longitudinal axis.

Example 36. The delivery assembly of any example herein, particularly any one of examples 1 to 35, wherein the pull-wire is coupled to an actuator of a handle of the delivery apparatus, wherein the actuator is configured to apply tension to the pull-wire.

Example 37. The delivery assembly of any example herein, particularly example 36, wherein the actuator comprises a rotatable knob.

Example 38. The delivery assembly of any example herein, particularly any one of examples 1 to 37, wherein tension applied to the pull-wire is configured to bend a distal portion of the steerable delivery catheter in a three-dimensional out-of-plane manner.

Example 39. The delivery assembly of any example herein, particularly any one of examples 1 to 38, wherein the steerable delivery catheter further comprises a polymeric layer disposed around the primary lumen.

Example 40. The delivery assembly of any example herein, particularly example 39, wherein the slotted tube is embedded within the polymeric layer.

Example 41. The delivery assembly of any example herein, particularly any one of examples 39 to 40, wherein the pull-wire lumen is embedded within the polymeric layer.

Example 42. The delivery assembly of any example herein, particularly any one of examples 39 to 41, wherein the slotted tube is disposed radially outward from the pull-wire lumen along at least a portion of the pull-wire lumen.

Example 43. The delivery assembly of any example herein, particularly any one of examples 39 to 42, wherein the steerable delivery catheter further comprises a braid.

Example 44. The delivery assembly of any example herein, particularly example 43, wherein the braid is embedded within the polymeric layer.

Example 45. The delivery assembly of any example herein, particularly example 43 or 44, wherein the braid is disposed between the pull-wire lumen and the slotted tube.

Example 46. The delivery assembly of any example herein, particularly any one of examples 39 to 45, wherein the polymeric layer comprises an encapsulating polymeric layer, and an outer polymeric layer disposed around the encapsulating polymeric layer.

Example 47. The delivery assembly of any example herein, particularly example 46, wherein the encapsulating polymeric layer has a non-uniform cross-sectional thickness, and wherein the pull-wire lumen extends through a thicker wall section of the encapsulating polymeric layer.

Example 48. The delivery assembly of any example herein, particularly any one of examples 39 to 47, wherein the steerable delivery catheter further comprises a pull ring, and wherein the pull-wire is coupled to the pull-wing.

Example 49. The delivery assembly of any example herein, particularly example 48, wherein the pull ring is embedded within the polymeric layer.

Example 50. The delivery assembly of any example herein, particularly example 48 or 49, wherein the pull ring is coupled to the slotted tube.

Example 51. The delivery assembly of any example herein, particularly example 50, wherein the pull ring comprises at least one cut-out, and wherein the slotted tube further comprises at least one tube teeth received within the at least one cut-out.

Example 52. The delivery assembly of any example herein, particularly example 48 or 49, wherein the pull ring is integrally formed with the slotted tube.

Example 53. The delivery assembly of any example herein, particularly any one of examples 48 to 52, wherein the pull ring comprises one or more windows through which the polymeric layer radially extends.

Example 54. The delivery assembly of any example herein, particularly any one of examples 48 to 53, wherein the pull ring comprises a wire groove through which the pull-wire extends.

Example 55. The delivery assembly of any example herein, particularly example 54, wherein the slotted tube comprises a semi-circular cut-out aligned with the wire groove.

Example 56. The delivery assembly of any example herein, particularly any one of examples 39 to 46, wherein the polymeric layer comprises a polymeric material selected from at least one of: polyamides and polyether block amides.

Example 57. The delivery assembly of any example herein, particularly any one of examples 39 to 46, wherein the polymeric layer comprises:
a first polymer having a first stiffness along the distal segment of the steerable delivery catheter;
a second polymer having a second stiffness along an intermediate segment of the steerable delivery catheter; and
a third polymer having a third stiffness along a proximal segment of the steerable delivery catheter.

Example 58. The delivery assembly of any example herein, particularly example 57, wherein the first stiffness is less than the second stiffness.

Example 59. The delivery assembly of any example herein, particularly example 57 or 58, wherein the second stiffness is less than the third stiffness.

Example 60. The delivery assembly of any example herein, particularly any one of examples 39 to 59, wherein the steerable delivery catheter further comprises a primary lumen liner around the primary lumen, wherein the primary lumen liner is radially internal to the polymeric layer.

Example 61. The delivery assembly of any example herein, particularly example 60, wherein the primary lumen liner comprises polytetrafluoroethylene.

Example 62. The delivery assembly of any example herein, particularly any one of examples 39 to 61, wherein the steerable delivery catheter further comprises a pull-wire lumen liner around the pull-wire lumen.

Example 63. The delivery assembly of any example herein, particularly example 62, wherein the pull-wire lumen liner comprises polytetrafluoroethylene.

Example 64. The delivery assembly of any example herein, particularly any one of examples 39 to 61, the steerable delivery catheter further comprises a tip extending between the slotted tube and a catheter distal end.

Example 65. The delivery assembly of any example herein, particularly example 64, wherein the distal non-steerable portion has a length which is greater than a diameter defined by the primary lumen.

Example 66. The delivery assembly of any example herein, particularly any one of examples 1 to 65, wherein the delivery apparatus further comprises an inner catheter axially movable through the primary lumen.

Example 67. The delivery assembly of any example herein, particularly example 66, wherein the delivery apparatus further comprises a perforating device, the perforating device comprising:
a perforating member disposed within the inner catheter, wherein the perforating member configured to pierce a host leaflet of a host valvular structure to form a pilot puncture in the host leaflet; and
an expansion member supported by a distal end portion of the inner catheter, wherein the expansion member is configured to be inserted within the pilot puncture and to be selectively transitioned between a radially compressed configuration and a radially expanded configuration.

Example 68. The delivery assembly of any example herein, particularly example 67, wherein transitioning the expansion member to the radially expanded configuration, while positioned within the pilot puncture, is configured to expand the pilot puncture to form a leaflet opening.

Example 69. The delivery assembly of any example herein, particularly example 67 or 68, wherein the perforating member comprises a distal end portion configured to be positioned distal to the expansion member for formation of the pilot puncture.

Example 70. The delivery assembly of any example herein, particularly example 69, wherein the distal end portion of the perforating member is axially movable relative to the expansion member.

Example 71. The delivery assembly of any example herein, particularly example 69 or 70, wherein the distal end portion of the perforating member terminates at an angled surface.

Example 72. The delivery assembly of any example herein, particularly any one of examples 69 to 71, wherein the perforating member comprises a needle.

Example 73. The delivery assembly of any example herein, particularly example 72, wherein the needle is one or both of a spring-loaded needle and a Veress needle.

Example 74. The delivery assembly of any example herein, particularly any one of examples 67 to 73, further comprising a guidewire extending through the perforating member.

Example 75. The delivery assembly of any example herein, particularly example 74, wherein the guidewire comprises a sharp tip configured to penetrate through the host leaflet.

Example 76. The delivery assembly of any example herein, particularly example 74, further comprising an RF energy source coupled to the guidewire and configured to provide RF energy to a tip of the guidewire.

Example 77. The delivery assembly of any example herein, particularly example 67 or 68, wherein the perforating member is a guidewire extending through the inner catheter.

Example 78. The delivery assembly of any example herein, particularly example 77, further comprising an RF energy source coupled to the guidewire and configured to provide RF energy to a tip of the guidewire.

Example 79. The delivery assembly of any example herein, particularly example 77, wherein the guidewire comprises a sharp tip configured to penetrate through the host leaflet.

Example 80. The delivery assembly of any example herein, particularly any one of examples 68 to 79, wherein the expansion member is an inflatable balloon, and wherein the inner catheter is a balloon catheter.

Example 81. The delivery assembly of any example herein, particularly any one of examples 68 to 80, wherein the delivery apparatus further comprises a dilator shaft extending through the inner catheter, and a dilator attached to the dilator shaft.

Example 82. The delivery assembly of any example herein, particularly example 81, wherein the dilator is distal to the expansion member.

Example 83. The delivery assembly of any example herein, particularly example 81 or 82, wherein the perforating member is axially movable relative to the dilator shaft.

Example 84. The delivery assembly of any example herein, particularly any one of examples 81 to 83, wherein the dilator is a nosecone, and wherein the dilator shaft is a nosecone shaft.

Example 85. The delivery assembly of any example herein, particularly any one of examples 68 to 84, wherein the host valvular structure is a native valvular structure of native heart valve.

Example 86. The delivery assembly of any example herein, particularly any one of examples 68 to 84, wherein the host valvular structure is a valvular structure of previously implanted prosthetic valve that is implanted within a native heart valve.

Example 87. The delivery assembly of any example herein, particularly any one of examples 1 to 85, further comprising a prosthetic valve comprising a frame movable between a radially compressed and a radially expanded configuration.

Example 88. The delivery assembly of any example herein, particularly any one of examples 1 to 85, wherein the slot width of at least some of the slots gradually changes along a length of the slotted tube.

Example 89. The delivery assembly of any example herein, particularly any one of examples 1 to 85, wherein the slot width of at least some subsequent slots of the plurality of slots gradually increases in the distal direction.

Example 90. The delivery assembly of any example herein, particularly any one of examples 1 to 89, wherein one or more of the ribs comprises a protrusion extending towards a complementary recess formed in an adjacent one of the ribs.

Example 91. The delivery assembly of any example herein, particularly example 90, wherein the protrusion is positioned at the slot circumferential center of the corresponding slot.

Example 92. A method for modifying a steerable delivery catheter, the method comprising:
providing a delivery catheter, which comprises:
   a primary lumen defining a central longitudinal axis;
   a polymeric layer disposed around the primary lumen;
   a slotted tube embedded in the polymeric layer and disposed around the primary lumen along at least a distal segment of the steerable delivery catheter, the slotted tube comprising a plurality of slots axially spaced from each other, wherein each slot defines a slot circumferential center between circumferential ends of the slot, wherein a distal-most slot is circumferentially aligned with an adjacent slot along the length of the slotted tube;
holding the polymeric layer at a first holding point and at a second holding point, wherein the first holding point is axially spaced from the second holding point, wherein the second holding point is in mechanical communication with a distal end portion of the slotted tube and the first holding point is in mechanical communication with a proximal end and/or an intermediate portion of the slotted tube;
imparting energy to a portion of the polymeric layer, which is in mechanical communication with the slotted tube, thereby increasing the malleability of the portion of the polymeric layer; and
rotating one of the first and second holding points around the central longitudinal axis relative to the other holding point, thereby twisting the polymeric layer and slotted tube radially around the central longitudinal axis, so that the circumferential center of the distal-most slot is circumferentially offset from the circumferential center of the adjacent slot by a helix angle.

Example 93. The method of any example herein, particularly example 92, wherein at least 25% of the slots of the slotted tube of the provided delivery catheter are circumferentially aligned with each other along the length of the slotted tube.

Example 94. The method of any example herein, particularly any one of examples 92 to 93, wherein at least 50% of the slots of the slotted tube of the provided delivery catheter are circumferentially aligned with each other along the length of the slotted tube

Example 95. The method of any example herein, particularly any one of examples 92 to 94, wherein at least 75% of the slots of the slotted tube of the provided delivery catheter are circumferentially aligned with each other along the length of the slotted tube.

Example 96. The method of any example herein, particularly any one of examples 92 to 95, each one of the slots of the slotted tube of the provided delivery catheter are circumferentially aligned with each other along the length of the slotted tube.

Example 97. The method of any example herein, particularly any one of examples 92 to 96, wherein each one of the slots of the slotted tube of the provided delivery catheter are circumferentially aligned with each other along the length of the slotted tube.

Example 98. The method of any example herein, particularly any one of examples 92 to 97, wherein the slotted tube of the provided delivery catheter comprises a plurality of ribs defined between the slots.

Example 99. The method of any example herein, particularly any one of examples 92 to 98, wherein each slot of the slotted tube of the provided delivery catheter has a slot width.

Example 100. The method of any example herein, particularly any one of examples 92 to 99, wherein the provided delivery catheter further comprises a pull-wire lumen extending along at least a portion of the steerable delivery catheter.

Example 101. The method of any example herein, particularly any one of examples 92 to 100, wherein the provided delivery catheter further comprises a pull-wire slidingly extending through the pull-wire lumen and attached to the slotted tube.

Example 102. The method of any example herein, particularly any one of examples 92 to 101, wherein each slot spans of the slotted tube of the provided delivery catheter spans more than 180° of the circumference of the slotted tube.

Example 103. The method of any example herein, particularly any one of examples 92 to 101, wherein each slot spans of the slotted tube of the provided delivery catheter spans more than 220° of the circumference of the slotted tube.

Example 104. The method of any example herein, particularly any one of examples 92 to 101, wherein each slot spans of the slotted tube of the provided delivery catheter spans more than 270° of the circumference of the slotted tube.

Example 105. The method of any example herein, particularly any one of examples 92 to 104, wherein the helix angle is not greater than 15°.

Example 106. The method of any example herein, particularly any one of examples 92 to 104, wherein the helix angle is not greater than 10°.

Example 107. The method of any example herein, particularly any one of examples 92 to 104, wherein the helix angle is not greater than 5°.

Example 108. The method of any example herein, particularly example 101, wherein upon the rotation the pull-wire is aligned with the slot circumferential centers.

Example 109. The method of any example herein, particularly example 100, wherein upon the rotation the pull-wire lumen is aligned with the slot circumferential centers.

Example 110. The method of any example herein, particularly any one of examples 92 to 109, wherein the slotted tube of the provided delivery catheter further comprises a backbone opposite to the slot circumferential centers, defined by uncut portions of the slotted tube between the circumferential ends of the slots.

Example 111. The method of any example herein, particularly example 110, wherein the pull-wire extends opposite to the backbone.

Example 112. The method of any example herein, particularly example 110 of 111, wherein the pull-wire lumen extends opposite to the backbone.

Example 113. The method of any example herein, particularly any one of examples 110 to 112, wherein the slotted tube further comprises a plurality of opposite cuts axially spaced from each other and extending through the backbone.

Example 114. The method of any example herein, particularly example 113, wherein the opposite cuts are axially disposed between the slots.

Example 115. The method of any example herein, particularly example 113 or 114, wherein each opposite cut comprises an opening and two slits circumferentially extending therefrom.

Example 116. The method of any example herein, particularly example 115, wherein the openings of the opposite cuts are positioned circumferentially opposite to the slot circumferential centers.

Example 117. The method of any example herein, particularly example 116, wherein the pull-wire extends opposite to the openings of the opposite cuts.

Example 118. The method of any example herein, particularly example 116 or 117, wherein the pull-wire lumen extends opposite to the openings of the opposite cuts.

Example 119. The method of any example herein, particularly any one of examples 92 to 118, wherein a slot width of at least one of the plurality of slots of the slotted tube of the provided delivery catheter is increased in size towards the slot circumferential center.

Example 120. The method of any example herein, particularly any one of examples 92 to 118, wherein a slot width of at least one of the plurality of slots of the slotted tube of the provided delivery catheter is uniform between the circumferential ends of the slot.

Example 121. The method of any example herein, particularly any one of examples 92 to 120, wherein the slots of the slotted tube of the provided delivery catheter comprise distal slots along a distal portion of the slotted tube, and intermediate slots along an intermediate portion of the slotted tube.

Example 122. The method of any example herein, particularly example 121, wherein the slots further comprise proximal slots along a proximal portion of the slotted tube.

Example 123. The method of any example herein, particularly example 122, wherein the shape of the distal slots is different from the shape of the intermediate slots and/or the proximal slots.

Example 124. The method of any example herein, particularly example 123, wherein a slot width of each slot of the distal slots and the intermediate slots increases towards the corresponding slot circumferential center.

Example 125. The method of any example herein, particularly example 123 or 124, wherein the slot width of each slot of the proximal slots is uniform between the corresponding circumferential ends.

Example 126. The method of any example herein, particularly example 122, wherein the shape of the distal slots is different from the shape of at least one of the intermediate slots or the proximal slots.

Example 127. The method of any example herein, particularly any one of examples 122 to 126, wherein each slot defines a slot length between its circumferential ends, and wherein the slot length of the distal slots is different from the slot length of the intermediate slots and/or the proximal slots.

Example 128. The method of any example herein, particularly any one of examples 122 to 127, wherein the width of the ribs disposed between the distal slots is different from the width of the ribs disposed between the intermediate slots and/or the proximal slots.

Example 129. The method of any example herein, particularly example 128, wherein the ribs between the distal slots are narrower than the ribs between the intermediate slots.

Example 130. The method of any example herein, particularly example 128 or 129, wherein the ribs between the intermediate slots are narrower than the ribs between the proximal slots.

Example 131. The method of any example herein, particularly any one of examples 122 to 130, wherein the helix angle comprises:
a distal helix angle by which the distal slots are angularly offset from each other;
an intermediate helix angle by which the intermediate slots are angularly offset from each other; and
a proximal helix angle by which the distal slots are angularly offset from each other.

Example 132. The method of any example herein, particularly example 131, wherein the distal helix angle is different from at least one of the intermediate helix angle and/or the proximal helix angle.

Example 133. The method of any example herein, particularly example 131 or 132, wherein a distal-most slot of the intermediate slots is angularly offset from a proximal-most slot of the distal slots by an angle between the distal helix angle and the intermediate helix angle, inclusive.

Example 134. The method of any example herein, particularly any one of examples 131 to 133, wherein a distal-most slot of the proximal slots is angularly offset from a proximal-most slot of the intermediate slots by an angle between the intermediate helix angle and the proximal helix angle, inclusive.

Example 135. The method of any example herein, particularly any one of examples 92 to 134, wherein the slots upon the rotation are orthogonal to an angled axis, defined as an axis which is angled at the helix angle relative to the central longitudinal axis.

Example 136. The method of any example herein, particularly example 101, wherein the pull-wire is coupled to an actuator of a handle of the delivery apparatus, wherein the actuator is configured to apply tension to the pull-wire.

Example 137. The method of any example herein, particularly example 136, wherein the actuator comprises a rotatable knob.

Example 138. The method of any example herein, particularly example 101, wherein tension applied to the pull-wire is configured to bend a distal portion of the steerable delivery catheter in a three-dimensional out-of-plane manner.

Example 139. The method of any example herein, particularly any one of examples 92 to 138, wherein the polymeric layer has a temperature-dependent malleability.

Example 140. The method of any example herein, particularly any one of examples 92 to 139, wherein the polymeric layer comprises at least one polymer having a glass transition temperature in the range of 50° C. to 250° C.

Example 141. The method of any example herein, particularly any one of examples 92 to 140, wherein the pull-wire lumen is embedded within the polymeric layer.

Example 142. The method of any example herein, particularly any one of examples 92 to 41, wherein the slotted tube is disposed radially outward from the pull-wire lumen along at least a portion of the pull-wire lumen.

Example 143. The method of any example herein, particularly any one of examples 92 to 142, wherein the steerable delivery catheter further comprises a braid.

Example 144. The method of any example herein, particularly example 143, wherein the braid is embedded within the polymeric layer.

Example 145. The method of any example herein, particularly example 143 or 144, wherein the braid is disposed between the pull-wire lumen and the slotted tube.

Example 146. The method of any example herein, particularly any one of examples 139 to 145, wherein the polymeric layer comprises an encapsulating polymeric layer, and an outer polymeric layer disposed around the encapsulating polymeric layer.

Example 147. The method of any example herein, particularly example 146, wherein the encapsulating polymeric layer has a non-uniform cross-sectional thickness, and wherein the pull-wire lumen extends through a thicker wall section of the encapsulating polymeric layer.

Example 148. The method of any example herein, particularly any one of examples 139 to 147, wherein the steerable delivery catheter further comprises a pull ring, and wherein the pull-wire is coupled to the pull-wing.

Example 149. The method of any example herein, particularly example 148, wherein the pull ring is embedded within the polymeric layer.

Example 150. The method of any example herein, particularly example 148 or 149, wherein the pull ring is coupled to the slotted tube.

Example 151. The method of any example herein, particularly example 150, wherein the pull ring comprises at least one cut-out, and wherein the slotted tube further comprises at least one tube teeth received within the at least one cut-out.

Example 152. The method of any example herein, particularly example 148 or 149, wherein the pull ring is integrally formed with the slotted tube.

Example 153. The method of any example herein, particularly any one of examples 148 to 152, wherein the pull ring comprises one or more windows through which the polymeric layer radially extends.

Example 154. The method of any example herein, particularly any one of examples 148 to 153, wherein the pull ring comprises a wire groove through which the pull-wire extends.

Example 155. The method of any example herein, particularly example 154, wherein the slotted tube comprises a semi-circular cut-out aligned with the wire groove.

Example 156. The method of any example herein, particularly any one of examples 139 to 146, wherein the polymeric layer comprises at least one polymeric material selected from: polyamides and polyether block amides.

Example 157. The method of any example herein, particularly any one of examples 92 to 146, wherein the polymeric layer comprises:
a first polymer having a first stiffness along the distal segment of the steerable delivery catheter;
a second polymer having a second stiffness along an intermediate segment of the steerable delivery catheter; and
a third polymer having a third stiffness along a proximal segment of the steerable delivery catheter.

Example 158. The method of any example herein, particularly example 157, wherein the first stiffness is less than the second stiffness.

Example 159. The method of any example herein, particularly example 157 or 158, wherein the second stiffness is less than the third stiffness.

Example 160. The method of any example herein, particularly any one of examples 92 to 159, wherein the steerable delivery catheter further comprises a primary lumen liner around the primary lumen, wherein the primary lumen liner is radially internal to the polymeric layer.

Example 161. The method of any example herein, particularly example 160, wherein the primary lumen liner comprises polytetrafluoroethylene.

Example 162. The method of any example herein, particularly any one of examples 139 to 161, wherein the steerable delivery catheter further comprises a pull-wire lumen liner around the pull-wire lumen.

Example 163. The method of any example herein, particularly example 162, wherein the pull-wire lumen liner comprises polytetrafluoroethylene.

Example 164. The method of any example herein, particularly any one of examples 139 to 61, the steerable delivery catheter further comprises a tip extending between the slotted tube and a catheter distal end.

Example 165. The method of any example herein, particularly example 164, wherein the distal non-steerable portion has a length which is greater than a diameter defined by the primary lumen.

Example 166. The method of any example herein, particularly any one of examples 92 to 165, wherein a slot width of at least some of the slots of the slotted tube of the provided delivery catheter gradually changes along a length of the slotted tube.

Example 167. The method of any example herein, particularly any one of examples 92 to 166, wherein a slot width of at least some subsequent slots of the plurality of slots gradually increases in the distal direction.

Example 168. The method of any example herein, particularly example 98, wherein one or more of the ribs comprises a protrusion extending towards a complementary recess formed in an adjacent one of the ribs.

Example 169. The method of any example herein, particularly example 168, wherein the protrusion is positioned at the slot circumferential center of the corresponding slot.

Example 170. The method of any example herein, particularly any one of examples 92 to 169, which comprises holding the polymeric layer only at the first holding point and at the second holding point.

Example 171. The method of any example herein, particularly any one of examples 92 to 170, wherein at least part of a length of the slotted tube is axially disposed between the first holding point and the second holding point.

Example 172. The method of any example herein, particularly example 171, wherein at least 50% of the length of the slotted tube is axially disposed between the first holding point and the second holding point.

Example 173. The method of any example herein, particularly any one of examples 171 to 172, wherein at least 60% of the length of the slotted tube is axially disposed between the first holding point and the second holding point.

Example 174. The method of any example herein, particularly any one of examples 171 to 173, wherein at least 70% of the length of the slotted tube is axially disposed between the first holding point and the second holding point.

Example 175. The method of any example herein, particularly any one of examples 171 to 174, wherein at least 80% of the length of the slotted tube is axially disposed between the first holding point and the second holding point.

Example 176. The method of any example herein, particularly any one of examples 171 to 175, wherein at least 90% of the length of the slotted tube is axially disposed between the first holding point and the second holding point.

Example 177. The method of any example herein, particularly any one of examples 171 to 176, wherein the entire length of the slotted tube is axially disposed between the first holding point and the second holding point.

Example 178. The method of any example herein, particularly any one of examples 92 to 177, wherein the distal end portion of the slotted tube is embedded within the polymeric layer proximally to the second holding point.

Example 179. The method of any example herein, particularly any one of examples 92 to 178, wherein an axial distance between the first holding point and the second holding point is at least 500% greater than an axial distance between the second holding point and the distal end portion of the slotted tube.

Example 180. The method of any example herein, particularly example 179, wherein the axial distance between the first holding point and the second holding point is at least 1000% greater than the axial distance between the second holding point and the distal end portion of the slotted tube.

Example 181. The method of any example herein, particularly any one of examples 179 to 180, wherein the axial distance between the first holding point and the second holding point is at least 5000% greater than the axial distance between the second holding point and the distal end portion of the slotted tube.

Example 182. The method of any example herein, particularly any one of examples 179 to 181, wherein the axial distance between the first holding point and the second holding point is at least 10000% greater than the axial distance between the second holding point and the distal end portion of the slotted tube.

Example 183. The method of any example herein, particularly any one of examples 92 to 182, wherein the first holding point is in mechanical communication with the proximal end portion of the slotted tube.

Example 184. The method of any example herein, particularly any one of examples 92 to 183, wherein the proximal end portion of the slotted tube is embedded within the polymeric layer proximally to the first holding point.

Example 185. The method of any example herein, particularly any one of examples 92 to 184, wherein an axial distance between the first holding point and the second holding point is at least 500% greater than an axial distance between the first holding point and the proximal end portion of the slotted tube.

Example 186. The method of any example herein, particularly example 185, wherein the axial distance between the first holding point and the second holding point is at least 1000% greater than the axial distance between the first holding point and the proximal end portion of the slotted tube.

Example 187. The method of any example herein, particularly any one of examples 185 to 186, wherein the axial distance between the first holding point and the second holding point is at least 5000% greater than the axial distance between the first holding point and the proximal end portion of the slotted tube.

Example 188. The method of any example herein, particularly any one of examples 185 to 187, wherein the axial distance between the first holding point and the second holding point is at least 10000% greater than the axial distance between the first holding point and the proximal end portion of the slotted tube.

Example 189. The method of any example herein, particularly any one of examples 92 to 188, which further comprises preventing from the delivery catheter to substantially collapse inwards upon the holding at the first holding point.

Example 190. The method of any example herein, particularly any one of examples 92 to 188, which further comprises preventing from the delivery catheter to substantially collapse inwards upon the holding at the second point.

Example 191. The method of any example herein, particularly any one of examples 92 to 188, which further comprises maintaining at least one rod within the primary lumen.

Example 192. The method of any example herein, particularly example 191, wherein the at least one rod has a first holding point, wherein the method comprises maintaining the rod first holding point laterally substantially in parallel to the polymeric layer first holding point.

Example 193. The method of any example herein, particularly example 192, wherein holding the polymeric layer at the first holding point includes applying mechanical pressure on the polymeric layer first holding point radially inward, thereby also applying mechanical pressure on the rod first holding point radially inward.

Example 194. The method of any example herein, particularly any one of examples 191 to 193, wherein the at least one rod has a second holding point, wherein the method comprises maintaining the rod second holding point laterally substantially in parallel to the polymeric layer first holding point.

Example 195. The method of any example herein, particularly example 194, wherein holding the polymeric layer at the second holding point includes applying mechanical pressure on the polymeric layer second holding point radially inward, thereby also applying mechanical pressure on the rod second holding point radially inward.

Example 196. The method of any example herein, particularly any one of examples 191 to 195, wherein the method comprises maintaining a first rod and a second rod within the primary lumen.

Example 195. The method of any example herein, particularly example 194, wherein the method comprises maintaining a first rod and a second rod substantially axially aligned within the primary lumen.

Example 196. The method of any example herein, particularly any one of examples 194 to 195, wherein the first rod has a proximal end portion and a distal end portion, wherein the second rod has a proximal end portion and a distal end portion, and wherein the method comprises maintaining the first rod distal end portion adjacent to the second rod proximal end portion.

Example 197. The method of any example herein, particularly example 196, wherein the first rod distal end portion is in the form of a protrusion and the second rod proximal end portion is in the form of a recess, or wherein the first rod distal end portion is in the form of a recess and the second rod proximal end portion is in the form of a protrusion, and wherein the protrusion is inserted within the recess to maintain substantial axial alignment between the first rod and the second rod.

Example 198. The method of any example herein, particularly example 196, wherein each one of the protrusion and the recess is round shaped, thereby enabling relative rotation between the first rod and the second rod.

Example 199. The method of any example herein, particularly any one of examples 92 to 198, wherein imparting energy to the portion of the polymeric layer includes elevating the temperature of the portion of the polymeric layer.

Example 200. The method of any example herein, particularly example 199, wherein elevating the temperature of the portion of the polymeric layer includes applying external heat to the portion of the polymeric layer using a heating device.

Example 201. The method of any example herein, particularly example 199, wherein the heating device comprises a heat blower.

Example 202. The method of any example herein, particularly any one of examples 92 to 199, wherein imparting energy to the portion of the polymeric layer includes irradiating the portion of the polymeric layer.

Example 203. The method of any example herein, particularly example 199, wherein elevating the temperature of the portion of the polymeric layer includes irradiating the portion of the polymeric layer.

Example 204. The method of any example herein, particularly any one of examples 92 to 203, wherein imparting energy to the portion of the polymeric layer includes adjusting the temperature of the portion of the polymeric layer to a temperature T^{c}.

Example 205. The method of any example herein, particularly example 204, wherein temperature T^{c} is equal or above the glass transition temperature of at least one polymer comprised within the polymeric layer.

Example 206. The method of any example herein, particularly example 204, wherein temperature T^{c} is equal or above the glass transition temperature of the polymeric layer.

Example 207. The method of any example herein, particularly any one of examples 204 to 206, wherein temperature T^{c} is at least 35° C.

Example 208. The method of any example herein, particularly any one of examples 204 to 207, wherein temperature T^{c} is at least 50° C.

Example 209. The method of any example herein, particularly any one of examples 204 to 207, wherein temperature T^{c} is at least 70° C.

Example 210. The method of any example herein, particularly any one of examples 92 to 29, wherein the portion of the polymeric layer to which energy is imparted to is located between the first holding point and the second holding point of the polymeric layer.

Example 211. The method of any example herein, particularly example 210, wherein upon the imparting of the energy to the portion of the polymeric layer, a temperature T^{c} of the portion of the polymeric layer is higher than the temperature of the first holding point.

Example 212. The method of any example herein, particularly any one of examples 210 to 211, wherein upon the imparting of the energy to the portion of the polymeric layer, a temperature T^{c} of the portion of the polymeric layer is higher than the temperature of the second holding point.

Example 213. The method of any example herein, particularly any one of examples 92 to 212, wherein upon the imparting of the energy to the portion of the polymeric layer, the portion of the polymeric layer becomes at least partially malleable.

Example 214. The method of any example herein, particularly any one of examples 92 to 212, wherein imparting of the energy to the portion of the polymeric layer includes increasing the malleability of the portion of the polymeric layer.

Example 215. The method of any example herein, particularly any one of examples 92 to 214, wherein imparting energy to a portion of the polymeric layer and rotating one of the first and second holding points around the central longitudinal axis relative to the other holding point, are performed at least partially simultaneously.

Example 216. The method of any example herein, particularly any one of examples 92 to 214, wherein the method comprises adjusting the temperature of the portion of the polymeric layer to a temperature T^{c}, which is elevated compared to the ambient temperature, and maintaining the portion of the polymeric layer at the elevated temperature through the step of rotating one of the first and second holding points around the central longitudinal axis relative to the other holding point.

Example 217. The method of any example herein, particularly example 216, wherein the elevated temperature is at least T^{c}−10° C.

Example 218. The method of any example herein, particularly example 216, wherein the elevated temperature is about T^{c}.

Example 219. The method of any example herein, particularly any one of examples 92 to 218, wherein rotating one of the first and second holding points around the central longitudinal axis relative to the other holding point includes rotating one of the first and second holding points around the central longitudinal axis and holding the other holding point static.

Example 220. The method of any example herein, particularly any one of examples 92 to 219, which comprises rotating the second holding point around the central longitudinal axis.

Example 221. The method of any example herein, particularly any one of examples 92 to 220, which comprises rotating the second holding point around the central longitudinal axis and holding the first holding point static.

Example 222. The method of any example herein, particularly any one of examples 92 to 221, which comprises synchronically twisting the polymeric layer and slotted tube radially.

Example 223. The method of any example herein, particularly any one of examples 92 to 222, wherein the provided delivery catheter comprises a pull-wire lumen, which has a portion extending axially at least between the first holding point and the second holding point of the polymeric layer.

Example 224. The method of any example herein, particularly example 223, which comprises twisting the pull-wire lumen portion radially around the central longitudinal axis.

Example 225. The method of any example herein, particularly example 224, which comprises synchronically twisting the pull-wire lumen portion and the polymeric layer radially around the central longitudinal axis.

Example 226. The method of any example herein, particularly example 224, wherein which comprises synchronically twisting the pull-wire lumen portion and the slotted tube radially around the central longitudinal axis.

Example 227. The method of any example herein, particularly example 224, which comprises synchronically twisting the pull-wire lumen portion, the polymeric layer and the slotted tube radially around the central longitudinal axis.

Example 228. The method of any example herein, particularly any one of examples 92 to 227, wherein the provided delivery catheter comprises a braid, which has a portion extending axially at least between the first holding point and the second holding point of the polymeric layer.

Example 229. The method of any example herein, particularly example 228, which comprises twisting the braid portion radially around the central longitudinal axis.

Example 230. The method of any example herein, particularly example 228, which comprises synchronically twisting the braid portion and the polymeric layer radially around the central longitudinal axis.

Example 231. The method of any example herein, particularly example 228, which comprises synchronically twisting the braid portion and the slotted tube radially around the central longitudinal axis.

Example 232. The method of any example herein, particularly example 228, which comprises synchronically twisting the braid portion, the polymeric layer and the slotted tube radially around the central longitudinal axis.

Example 233. The method of any example herein, particularly example 228, which comprises synchronically twisting the braid portion, the pull-wire lumen portion, the polymeric layer and the slotted tube radially around the central longitudinal axis.

Example 234. The method of any example herein, particularly any one of examples 92 to 229, which comprises
providing a twisting apparatus and holding the polymeric layer at a first holding point and at a second holding point; and
rotating one of the first and second holding points around the central longitudinal axis relative to the other holding point using the twisting apparatus.

Example 235. The method of any example herein, particularly any one of examples 92 to 234, which further comprises a step of lowering the temperature of the portion of the polymeric layer.

Example 236. The method of any example herein, particularly example 235, wherein lowering the temperature of the portion of the polymeric layer includes adjusting the temperature of the portion of the polymeric layer to a temperature T^{e}.

Example 237. The method of any example herein, particularly example 236, wherein temperature T^{e} is equal or below the glass transition temperature of at least one polymer comprised within the polymeric layer.

Example 238. The method of any example herein, particularly example 236, wherein temperature T^{e} is below the glass transition temperature of at least one polymer comprised within the polymeric layer.

Example 239. The method of any example herein, particularly example 236, wherein temperature T^{e} is equal or below the glass transition temperature of the polymeric layer.

Example 240. The method of any example herein, particularly example 236, wherein temperature T^{e} is below the glass transition temperature of the polymeric layer.

Example 241. The method of any example herein, particularly any one of examples 236 to 240, wherein temperature T^{e} is no more than 35° C.

Example 242. The method of any example herein, particularly any one of examples 236 to 240, wherein temperature T^{e} is about ambient temperature.

Example 243. The method of any example herein, particularly any one of examples 235 to 243, wherein the portion of the polymeric layer of which the temperature is lowered is located between the first holding point and the second holding point of the polymeric layer.

Example 244. The method of any example herein, particularly example 243, wherein upon the lowering of the temperature of the portion of the polymeric layer, a temperature T^{e} of the portion of the polymeric layer is similar to the temperature of the first holding point.

Example 245. The method of any example herein, particularly any one of examples 243 to 244, wherein upon the lowering of the temperature of the portion of the polymeric layer, a temperature T^{e} of the portion of the polymeric layer is similar to the temperature of the second holding point.

Example 246. The method of any example herein, particularly any one of examples 235 to 245, wherein lowering the temperature of the portion of the polymeric layer includes reducing the malleability of the portion of the polymeric layer.

Example 247. The method of any example herein, particularly any one of examples 235 to 246, wherein the cooling is maintained until lowering the temperature of the portion of the polymeric layer includes reducing the malleability of the portion of the polymeric layer.

Example 248. The method of any example herein, particularly any one of examples 235 to 246, wherein the cooling is performed at ambient temperature.

Example 249. The method of any example herein, particularly any one of examples 235 to 246, which comprises maintaining the portion of the polymeric layer at ambient temperature for a time period sufficient reduce the relative elongation of the polymeric layer by at least 5%.

Example 250. The method of any example herein, particularly any one of examples 235 to 246, which comprises immersing the polymeric layer in water at a temperature of no more than 35° C.

Example 251. The method of any example herein, particularly any one of examples 92 to 250, which comprises holding the polymeric layer at a third holding point, wherein the third holding point is located axially between the first holding point and the second holding point, wherein the third holding point is axially spaced from the second holding point and the third holding point, wherein the first holding point is in mechanical communication with a proximal end portion of the slotted tube, wherein the third holding point is in mechanical communication with an intermediate end portion of the slotted tube.

Example 252. The method of any example herein, particularly example 251, which comprises rotating one of the first and third holding points around the central longitudinal axis relative to the other holding point.

Example 253. The method of any example herein, particularly example 252, which comprises rotating one of the second and third holding points around the central longitudinal axis relative to the other holding point.

Example 254. The method of any example herein, particularly example 253, wherein rotating one of the second and third holding points around the central longitudinal axis relative to the other holding point, and rotating one of the first and third holding points around the central longitudinal axis relative to the other holding point, is performed in the same direction.

Example 255. The method of any example herein, particularly example 254, wherein rotating one of the second and third holding points around the central longitudinal axis relative to the other holding point, and rotating one of the first and third holding points around the central longitudinal axis relative to the other holding point, is performed at a different extent, so that a helix angle between the first and third holding points is different than a helix angle between the second and third holding points.

Example 256. The method of any example herein, particularly example 252, which further comprises rotating one of the second and first holding points around the central longitudinal axis relative to the other holding point, in addition to the rotating one of the first and third holding points around the central longitudinal axis relative to the other holding point.

Example 257. The method of any example herein, particularly example 256, wherein rotating one of the first and third holding points around the central longitudinal axis relative to the other holding point, and rotating one of the first and second holding points around the central longitudinal axis relative to the other holding point, is performed in the same direction, so that a helix angle between the first and third holding points is different than a helix angle between the second and third holding points.

Example 258. A delivery apparatus comprising: a steerable delivery catheter comprising: a primary lumen defining a central longitudinal axis; a primary lumen defining a central longitudinal axis; and a slotted tube disposed around the primary lumen along at least a distal segment of the steerable delivery catheter, the slotted tube comprising a plurality of slots; and wherein a circumferential center of each slot is circumferentially offset from a circumferential center of an adjacent slot by a helix angle.

Example 259. The delivery apparatus of any example herein, particularly example 258, wherein each of the plurality of slots has a slot width and defines the slot circumferential center between circumferential ends of the slot.

Example 260. The delivery apparatus of any example herein, particularly example 258 or 259, wherein the slotted tube comprises a plurality of ribs defined between the slots.

Example 261. The delivery apparatus of any example herein, particularly any one of examples 258 to 260, wherein the steerable delivery catheter comprises a pull-wire lumen extending along at least a portion of the steerable delivery catheter.

Example 262. The delivery apparatus of any example herein, particularly example 261, wherein the steerable delivery catheter comprises a pull-wire slidingly extending through the pull-wire lumen and attached to the slotted tube.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A delivery assembly comprising:
a delivery apparatus comprising:
a steerable delivery catheter comprising:
a primary lumen defining a central longitudinal axis;
a slotted tube disposed around the primary lumen along at least a distal segment of the steerable delivery catheter, the slotted tube comprising:
a plurality of slots axially spaced from each other, wherein each slot has a slot width and defines a slot circumferential center between circumferential ends of the slot; and
a plurality of ribs defined between the slots;
a pull-wire lumen extending along at least a portion of the steerable delivery catheter; and
a pull-wire slidingly extending through the pull-wire lumen and copupled to the slotted tube;
wherein the circumferential center of each slot is circumferentially offset from the circumferential center of an adjacent slot by a helix angle.

2. The delivery assembly of claim 1, wherein each slot spans more than 220° of the circumference of the slotted tube.

3. The delivery assembly of claim 1, wherein the helix angle is not greater than 10°.

4. The delivery assembly of claim 1, wherein the pull-wire is aligned with the slot circumferential centers.

5. The delivery assembly of claim 1, wherein the slots comprise distal slots along a distal portion of the slotted tube, and intermediate slots along an intermediate portion of the slotted tube.

6. The delivery assembly of claim 5, wherein the slots further comprise proximal slots along a proximal portion of the slotted tube.

7. The delivery assembly of claim 6, wherein the width of the ribs disposed between the distal slots is different from the width of the ribs disposed between the intermediate slots and/or the proximal slots.

8. The delivery assembly of claim 6, wherein the helix angle comprises:
a distal helix angle by which the distal slots are angularly offset from each other;
an intermediate helix angle by which the intermediate slots are angularly offset from each other; and
a proximal helix angle by which the distal slots are angularly offset from each other.

9. The delivery assembly of claim 8, wherein the distal helix angle is different from at least one of the intermediate helix angle and/or the proximal helix angle.

10. The delivery assembly of claim 1, wherein tension applied to the pull-wire is configured to bend a distal portion of the steerable delivery catheter in a three-dimensional out-of-plane manner.

11. The delivery assembly of claim 1, wherein the delivery apparatus further comprises an inner catheter axially movable through the primary lumen.

12. The delivery assembly of claim 11, wherein the delivery apparatus further comprises a perforating device, the perforating device comprising:
a perforating member disposed within the inner catheter, wherein the perforating member configured to pierce a host leaflet of a host valvular structure to form a pilot puncture in the host leaflet; and
an expansion member supported by a distal end portion of the inner catheter, wherein the expansion member is configured to be inserted within the pilot puncture and to be selectively transitioned between a radially compressed configuration and a radially expanded configuration.

13. The delivery assembly of claim 1, wherein the slot width of at least some subsequent slots of the plurality of slots gradually increases in the distal direction.

14. The delivery assembly of claim 1, wherein one or more of the ribs comprises a protrusion extending towards a complementary recess formed in an adjacent one of the ribs.

15. A method for modifying a steerable delivery catheter, the method comprising:
providing a delivery catheter, which comprises:
a primary lumen defining a central longitudinal axis;
a polymeric layer disposed around the primary lumen;
a slotted tube embedded in the polymeric layer and disposed around the primary lumen along at least a distal segment of the steerable delivery catheter, the slotted tube comprising a plurality of slots axially spaced from each other, wherein each slot defines a slot circumferential center between circumferential ends of the slot, wherein a distal-most slot is circumferentially aligned with an adjacent slot along the length of the slotted tube;
holding the polymeric layer at a first holding point and at a second holding point, wherein the first holding point is axially spaced from the second holding point, wherein the second holding point is in mechanical communication with a distal end portion of the slotted tube and the first holding point is in mechanical communication with a proximal end and/or an intermediate portion of the slotted tube;
imparting energy to a portion of the polymeric layer, which is in mechanical communication with the slotted tube, thereby increasing the malleability of the portion of the polymeric layer; and
rotating one of the first and second holding points around the central longitudinal axis relative to the other holding point, thereby twisting the polymeric layer and slotted tube radially around the central longitudinal axis, so that the circumferential center of the distal-most slot is circumferentially offset from the circumferential center of the adjacent slot by a helix angle.

16. The method of claim 15, wherein provided delivery catheter further comprises a pull-wire slidingly extending through a pull-wire lumen and coupled to the slotted tube; wherein upon the rotation the pull-wire is aligned with the slot circumferential centers; and wherein the provided delivery catheter further comprises a pull-wire lumen extending along at least a portion of the steerable delivery catheter, and wherein upon the rotation the pull-wire lumen is aligned with the slot circumferential centers.

17. The method of claim 15, wherein at least part of a length of the slotted tube is axially disposed between the first holding point and the second holding point, and wherein the entire length of the slotted tube is axially disposed between the first holding point and the second holding point.

18. The method of claim 15, further comprising: maintaining at least one rod within the primary lumen;
wherein the at least one rod has a first holding point, wherein the method comprises maintaining the rod's first holding point laterally substantially in parallel to the polymeric layer's first holding point;
wherein holding the polymeric layer's at the first holding point includes applying mechanical pressure on the polymeric layer's first holding point radially inward, thereby also applying mechanical pressure on the rod's first holding point radially inward;
wherein the at least one rod has a second holding point, wherein the method comprises maintaining the rod's second holding point laterally substantially in parallel to the polymeric layer's first holding point; and
wherein holding the polymeric layer's at the second holding point includes applying mechanical pressure on the polymeric layer's second holding point radially inward, thereby also applying mechanical pressure on the rod's second holding point radially inward.

19. The method of claim 15, wherein imparting energy to the portion of the polymeric layer includes adjusting the temperature of the portion of the polymeric layer to a temperature T^{e}, which is equal or above the glass transition temperature of the polymeric layer.

20. The method of claim 15, comprising synchronically twisting the polymeric layer and slotted tube radially.

21. A delivery apparatus comprising:
a steerable delivery catheter comprising:
a primary lumen defining a central longitudinal axis;
a slotted tube disposed around the primary lumen along at least a distal segment of the steerable delivery catheter, the slotted tube comprising a plurality of slots; and
wherein a circumferential center of each slot is circumferentially offset from a circumferential center of an adjacent slot by a helix angle.

22. The delivery apparatus of claim 21, wherein each of the plurality of slots has a slot width and defines the slot circumferential center between circumferential ends of the slot.

23. The delivery apparatus of claim 21, wherein the slotted tube comprises a plurality of ribs defined between the slots.

24. The delivery apparatus of claim 21, wherein the steerable delivery catheter comprises a pull-wire lumen extending along at least a portion of the steerable delivery catheter.

25. The delivery apparatus of claim 24, wherein the steerable delivery catheter comprises a pull-wire slidingly extending through the pull-wire lumen and coupled to the slotted tube.
